(19) United States
(12) Patent Application Publication
(10) Pub. No.: US 2022/0193414 A1
(43) Pub. Date: Jun. 23, 2022

(54) METHODS AND SYSTEMS FOR NEURAL REGULATION

(71) Applicant: RESHAPE LIFESCIENCES, INC., St. Paul, MN (US)

(72) Inventors: Jonathan James WAATAJA, Plymouth, MN (US); Raj NIHALANI, Irvine, CA (US)

(73) Assignee: RESHAPE LIFESCIENCES, INC., St, Paul, MN (US)

(21) Appl. No.: 17/604,309

(22) PCT Filed: Apr. 17, 2020
(86) PCT No.: PCT/US2020/028810

§ 371 Date: Oct. 15, 2021

Related U.S. Application Data
(60) Provisional application No. 62/920,216, filed on Apr. 18, 2019.

(51) Int. Cl. A61N 1/36; A61N 1/05; A61N 1/378

(52) U.S. Cl. CPC A61N 1/36053; A61N 1/36196; A61N 1/36175; A61N 1/36171; A61N 1/36057; A61N 1/3787; A61N 1/3606; A61N 1/0551

(57) ABSTRACT

Methods and systems for regulating nerve activity and/or treating conditions associated with disorder of blood glucose are disclosed. A method of downregulating activity by applying a high frequency alternating current electrical signal to a nerve in a subject is disclosed. A method of upregulating activity by applying a low frequency stimulation signal to a nerve in a subject is disclosed. A method of regulating nerve activity by applying a high frequency signal to a first nerve/organ and applying a low frequency stimulation signal to a second nerve/organ is disclosed. The application of the high frequency signal and the low frequency stimulation signal to separate nerves or nerve branches/fibers can be independent, simultaneous, concurrent, or in a coordinated fashion in therapy programs. Various signal parameters including the waveform, frequency, amplitude, active/inactive phases are described.

### CROSS-REFERENCE TO OTHER APPLICATIONS

This application is being filed on Apr. 17, 2020, as a PCT International Patent Application and claims the benefit of U.S. Patent Application Ser. No. 62/920,216, filed Apr. 18, 2019, the disclosure of which is hereby incorporated in its entirety.

### INTRODUCTION

Modulation of nerve activity is useful for the treatment of gastrointestinal conditions including obesity and other eating disorders, inflammatory conditions such as inflammatory bowel disease and pancreatitis, diabetes, and hypertension. Application of neural modulation in some circumstances can be accompanied by a loss of effectiveness. This loss of effectiveness can in part be due to compliance of the patient with charging of the implanted device and/or effects on the nerve. It is desirable to identify electrical signal therapies that can minimize loss of effectiveness and decrease energy requirements of the device.

In particular, there is a need for Type II Diabetes Mellitus (T2DM) treatments due to growth rate, increased risks of comorbidities and cost to the health care system. With current growth trends, the diabetic population will increase to 366 million people worldwide by 2030. Western nations will be seriously affected; by 2050, it has been estimated as many as 1 in 3 US citizens will be diabetic. Progression of T2DM significantly increases risks of stroke, myocardial infarction, microvascular events, and mortality. Diabetics face average medical expenditures directly attributable to the disease of $9,600/year.

Despite medication, surgery and diet, T2DM remains challenging to effectively treat. Effective treatment options that are adjustable to patient's compliance are highly desirable. It is known that the vagus nerve controls organ systems that regulate blood glucose. However, treatment of T2DM using electrical neural modulation of the vagus nerve showed mixed results. Neural modulation methods using electrical conduction blockade alone applied to the vagus nerve have demonstrated increased glycemic control; however, this was in the context of sustained weight loss making it non-ideal for many diabetics. Electrical stimulation of a single vagus nerve trunk, or branch have failed to increase glycemic control. Since the vagus nerve, and its branches, control multiple organ systems involved in blood glucose regulation, it is highly desirable for new modulation methods and systems to effectively regulate vagus nerve activity and to control blood glucose level.

Methods and Systems for Neural Regulation

In some aspects, the present disclosure generally relates to methods and systems regulating nerve activity of a subject. The present method generally comprise applying one or more high frequency alternating current (HFAC) electrical signals and/or one or more low frequency signals to downregulate and/or upregulate activity of one or more nerves of a subject.

In some aspects, the present disclosure relates to a method for downregulating or upregulating nerve activity of a subject comprising applying an electrical signal to a nerve or a nerve branch/fiber thereof or an organ, wherein the electrical signal is characterized by cycles with charge and recharge phases followed by inactive phases on the scale of microseconds, on the scale of milliseconds, and/or on the scale of minutes, wherein the electrical signal has high-frequency of at least about 200 Hz or more.

In some aspects, the present disclosure relates to a method for upregulating/stimulating nerve activity of a subject comprising applying a low frequency electrical signal to a nerve or a nerve branch/fiber or an organ, wherein the low frequency electrical signal is in a range from about 0.01 Hz to about 100 Hz, preferably from about 0.01 Hz to about 30 Hz.

In some aspects, the present disclosure relates to methods and systems for regulating nerve activity of a subject by combining a high frequency electrical signal applied to a nerve or a nerve branch/fiber or an organ and a low frequency stimulation signal applied to a separate nerve or a separate nerve branch/fiber or a separate organ. The high frequency signal has parameters to downregulate or block nerve activity, and the low frequency stimulation signal has parameters to upregulate or stimulate nerve activity.

The methods and systems disclosed in the present disclosure provide effective solutions to treating a condition associated with impaired blood glucose regulation such as diabetes, Type II Diabetes, or Type II Diabetes Mellitus (T2DM).

Traditional methods such as stimulation of vagus nerve fibers innervating the pancreas causes an increase in plasma insulin, however, glucose levels are either unchanged or increased. Vagus nerve stimulation-induced pancreatic secretion of glucagon may explain why glucose was not attenuated. Ligation of neuronal fibers innervating the liver has been shown to affect glucose possibly though disinhibition of vagal efferents innervating the pancreas, decreased hepatic sensitivity to glucagon and/or decreased insulin resistance through attenuation of PPARα. However, ligation is non-reversible, the body may adapt to ligation over time and significant unwanted side effects may be associated with this technique. Other known methods such as hepatic vagotomy could unfavorably decrease insulin levels and increase glucose level.

The present disclosure provides methods and systems to treat T2DM by combining pacing stimulation of celiac fibers innervating the pancreas along with reversible electrical blockade of neuronal hepatic fibers innervating the liver. It was surprisingly found that the present methods and systems compared to stand alone stimulation or stand alone ligation, resulted in a lower blood glucose level following an intravenous (IV) glucose tolerance test (IVGTT) during stimulation of the celiac branch of the vagus nerve while simultaneously using ligation or application of high frequency alternating current (HFAC) to the vagus nerve hepatic branch. It was also found that the present methods and systems effectively lowered the blood glucose level following an oral glucose tolerance test (OGTT) during stimulation of the celiac branch of the vagus nerve while simultaneously using ligation or application of high frequency alternating current (HFAC) to the vagus nerve hepatic branch in pig and porcine models of T2DM.

### SUMMARY OF DISCLOSURE

The present disclosure describes systems and methods providing electrical signal therapy for downregulating and/or upregulating nerve activity in a subject. In embodiments, the electrical signal therapy provides more than one microsecond cycle comprising more than one period, each period comprising charge and recharge phase which may or may not have pulse delays, each period having a frequency of about at least 200 Hz; and a microsecond inactive phase. In embodiments, more than one microsecond cycle forms a millisecond cycle, each millisecond cycle being separated by a millisecond inactive phase. The length of time of the microsecond and/or millisecond inactive phases provides for the ability to vary how often electrical signal treatment is applied to the nerve during an on time, provides for downregulation and/or upregulation of neural activity, and provides energy savings as compared to electrical signal therapy not having inactive phases. The electrical signal having a frequency of at least 200 Hz is characterized as a high frequency electrical signal in the present disclosure. High frequency signal is primarily used to downregulate or block nerve/neural activity.

In embodiments, a method of applying an electrical signal having parameters that downregulate and/or upregulate nerve activity to a nerve in a subject comprises: applying the electrical signal to the nerve during an on time, wherein the electrical signal comprises more than one microsecond cycle comprising: a) more than one period, each period comprising a charge and recharge phase and optionally, one or more pulse delays, each period having a frequency of at least 200 Hz; and b) a microsecond inactive phase. In embodiments, the microsecond inactive phase is longer than the period. In embodiments, the length of the inactive phase can vary between each period. In embodiments, the period is about 1000 microseconds or less. In embodiments, the microsecond inactive phase is in a ratio to the period of about 10 to 1, 8 to 1, 6 to 1, 4 to 1, or 2 to 1. In embodiments, the microsecond inactive phase is at least about 80 microseconds. In embodiments, the microsecond inactive phase is at least 80 microseconds up to 10,000 microseconds, 200 microseconds up to 10,000 microseconds, or 400 microseconds up to 10,000 microseconds.

In embodiments, the duty cycle for the microsecond cycle is about 75% or less. In embodiments, the duty cycle for the microsecond cycle is about 50% or less.

In embodiments, the frequency is at least 200 Hz, 300 Hz, 400 Hz, 500 Hz, 600 Hz. 700 Hz, 800 Hz, 900 Hz, 1000 Hz, 2000 Hz, 3000 Hz, 4000 Hz, 5000 Hz, 6000 Hz, 7000 Hz, 8000 Hz, 9000 Hz, 10,000 Hz, 11,000 Hz, 12,000 Hz, 13,000 Hz, 14,000 Hz, 15,000 Hz, 16,000 Hz, 17,000 Hz, 18,000 Hz, 19,000 Hz, 20,000 Hz, 21,000 Hz, 22.000 Hz. 23,000 Hz, 24,000 Hz, 25.000 Hz, 50,000 Hz, 60,000 Hz, 70,000 Hz, 80,000 Hz, 90,000 Hz, 100 kHz, 200 kHz. 250 kHz or more. In embodiments, electrical signals at such frequencies can downregulate nerve activity.

In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can upregulate or stimulate nerve activity.

In other embodiments, the method comprises applying an electrical signal to a nerve in a subject, wherein the electrical signal comprises more than one microsecond cycle to form a millisecond active phase, and applying more than one millisecond active phase during the on time, wherein each millisecond active phase is separated by a millisecond inactive phase during the on time. In embodiments, the millisecond inactive phase is longer than the millisecond active phase. In embodiments, the millisecond inactive phase can vary in time between each millisecond active phase.

In embodiments, the millisecond active phase is at least 0.16 milliseconds. In embodiments, the millisecond active phase is 0.16 millisecond to 1,100 milliseconds, 0.16 millisecond to 900 milliseconds, 0.16 millisecond to 800 milliseconds, 0.16 millisecond to 700 milliseconds, 0.16 millisecond to 600 milliseconds. 0.16 millisecond to 500 milliseconds, 0.16 to 400 milliseconds, 0.16 to 300 milliseconds, 0.16 to 200 milliseconds, 0.16 to 100 milliseconds, 0.16 to 50 milliseconds, 0.16 to 40 milliseconds, 0.16 to 30 milliseconds, 0.16 to 20 milliseconds, 0.16 to 10 milliseconds, or 0.16 to 5 milliseconds. In embodiments, the millisecond active phase is at least 1 millisecond. In other embodiments, the millisecond active phase is 1 to 1,100 milliseconds, 1 millisecond to 900 milliseconds, 1 millisecond to 800 milliseconds, 1 millisecond to 700 milliseconds, 1 millisecond to 600 milliseconds, 1 millisecond to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, 1 to 10 milliseconds, or 1 to 5 milliseconds.

In embodiments, the millisecond active phase comprises at least 2 to 100 microsecond cycles, at least 2 to 90, at least 2 to 80, at least 2 to 70, at least 2 to 60, at least 2 to 50, at least 2 to 40, at least 2 to 30, at least 2 to 20, at least 2 to 10, at least 2 to 5, or at least 2 to 4 microsecond cycles.

In embodiments, the millisecond inactive phase is in a ratio to the millisecond active phase of about 10 to 1, 8 to 1, 6 to 1, 4 to 1, 2 to 1 or 1 to 2. In embodiments, the millisecond inactive phase is at least 0.08 milliseconds. In embodiments, the millisecond inactive phase is 0.08 millisecond to 11,000 milliseconds, 0.08 millisecond to 9000 milliseconds, 0.08 millisecond to 8000 milliseconds, 0.08 millisecond to 7000 milliseconds, 0.08 millisecond to 6000 milliseconds, 0.08 millisecond to 5000 milliseconds, 0.08 to 4000 milliseconds, 0.08 to 3000 milliseconds, 0.08 to 2000 milliseconds, 0.08 to 1000 milliseconds, 0.08 to 500 milliseconds, 0.08 to 400 milliseconds, 0.08 to 300 milliseconds, 0.08 to 200 milliseconds, 0.08 to 100 milliseconds, 0.08 to 50 milliseconds, 0.08 to 40 milliseconds, 0.08 to 30 milliseconds, 0.08 to 20 milliseconds, or 0.08 to 10 milliseconds. In embodiments, the millisecond inactive phase is 1 millisecond to 11,000 milliseconds, 1 millisecond to 9000 milliseconds, 1 millisecond to 8000 milliseconds, 1 millisecond to 7000 milliseconds, 1 millisecond to 6000 milliseconds, 1 millisecond to 5000 milliseconds, 1 to 4000 milliseconds, 1 to 3000 milliseconds, 1 to 2000 milliseconds, 1 to 1000 milliseconds, 1 to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, or 1 to 10 milliseconds.

In yet other embodiments, a method of applying an electrical signal having parameters to downregulate and/or upregulate nerve activity to a nerve in a subject comprising: applying the electrical signal to the nerve during an on time, wherein the electrical signal comprises a first pattern comprising at least one microsecond cycle; and a second pattern comprising more than one millisecond active phase, wherein each millisecond active phase comprises more than one microsecond cycle, and each millisecond active phase is separated by a millisecond inactive phase. In embodiments, the first and second patterns have different amplitude. In embodiments, a ramp up and/or ramp down in amplitude is employed to shift the change in amplitude.

In embodiments, the microsecond cycle comprises at least one period, each period comprising a charge and recharge phase, and optionally, a pulse delay, wherein each period has a frequency of at least 200 Hz; and a microsecond inactive phase.

In embodiments, the first pattern has amplitude greater than the second pattern. In embodiments, the first and second patterns are separated by a ramp up and/or a ramp down of amplitude. In embodiments, the ratio of the amplitude of the first pattern to the amplitude of the second pattern is at least 10 to 1, 8 to 1, 6 to 1, 4 to 1, 2 to 1 or 4 to 3.

In some aspects, the present disclosure relates to a method for upregulating/stimulating nerve activity of a subject comprising applying a low frequency stimulation signal to a nerve or a nerve branch/fiber, wherein the low frequency electrical signal is in a range from about 0.01 Hz to about 100 Hz, preferably from about 0.01 Hz to about 30 Hz.

In embodiments, the stimulation signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. In embodiments, the stimulation signal further comprises at least one stimulation active phase, wherein each of the at least one stimulation active phase comprises at least one stimulation cycle. In these instances each of the at least one stimulation active phase may be separated by an idle. In embodiments, the pulse of the low frequency stimulation signal is monophasic, or biphasic, or combinations thereof. In embodiments, the low frequency stimulation signal has a biphasic pulse with a negative (cathodic) charge phase followed by a positive (anodic) charge phase within one pulse.

In embodiments, the pulse width of the stimulation signal is from about 50 microseconds to about 10,000 microseconds.

In embodiments, the stimulation inactive phase of the low frequency stimulation is from about 0.01 to about 100 seconds.

In embodiments, the pulse of the stimulation signal is monophasic pulse, or biphasic pulse, or combinations thereof.

In embodiments, the stimulation signal has an on time of about 30 seconds to about 30 minutes.

In embodiments, the stimulation signal has a current amplitude in a range from about 0.01 mAmps to about 20 mAmps. In embodiments, the stimulation signal has a voltage amplitude in a range from about 0.01 volts to about 20 volts.

In embodiments, the stimulation signal comprises an abrupt start of pulses, or a ramp up of current/voltage amplitude, or a ramp up of frequency, or a ramp up of pulse widths, or combination thereof at or near initiation of applying the stimulation signal.

In embodiments, the ramp up or ramp down time of current/voltage amplitude, frequency, or pulse widths of the stimulation signal is from about 10 seconds to about 15 minutes.

In embodiments, the ramp up or ramp down of the stimulation signal is linear or non-linear.

In some aspects, the present disclosure relates to methods and systems for regulating nerve activity of a subject by combining a high frequency electrical signal applied to a nerve branch/fiber and a low frequency stimulation signal applied to a separate nerve branch/fiber. The high frequency signal has parameters to downregulate or block nerve activity, and the low frequency stimulation signal has parameters to upregulate or stimulate nerve activity.

In some embodiments, the present disclosure relates to a method for regulating nerve activity of a subject comprising applying a first electrical signal to a first nerve branch/fiber and applying a second electrical signal to a second nerve branch/fiber. The first electrical signal downregulates nerve activity and has a frequency from about 200 Hz to about 100 kHz, whereas the second electrical signal upregulates nerve activity and has a frequency from about 0.01 Hz to 199 Hz. In embodiments, the first electrical signal and the second electrical signal are applied concurrently or simultaneously. In embodiments, the first electrical signal and the second electrical signal are applied at different times.

In embodiments, the first electrical signal comprises at least one microsecond cycle and optionally a microsecond inactive phase. Each of the at least one microsecond cycle comprises at least one period, each of the at least one period comprising a pulse comprising a charge recharge phase, the pulse having a pulse width, and wherein the second electrical signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. In embodiments, the pulse of the low frequency stimulation signal is monophasic, or biphasic, or combinations thereof. In embodiments, the low frequency stimulation has a biphasic pulse with a negative (cathodic) charge phase followed by a positive (anodic) charge phase within one pulse.

In embodiments, the first electrical signal further comprises at least one millisecond active phase, wherein each of the at least one millisecond active phase comprises at least one microsecond cycle, and wherein each of the at least one millisecond active phase is separated by a millisecond inactive phase. In embodiments, the second electrical signal further comprises at least one stimulation active phase, in at least these instance each of the at least one stimulation active phase comprises at least one stimulation active cycle, where each of the at least one stimulation active phase is separated by an idle.

In embodiments, the first electrical signal is low duty cycle of about 75% or less, or preferably 50% or less.

In embodiments, the pulse width of the first electrical signal is from about 10 microseconds to about 500 microseconds. In embodiments, the pulse width of the second electrical signal is from about 50 microseconds to about 10,000 microseconds.

In embodiments, the microsecond inactive phase of the first electrical signal is from about 0 to about 10,000 microseconds. In embodiments, the stimulation inactive phase of the second electrical signal is from about 0.01 to about 100 seconds.

In embodiments, the first electrical signal and the second electrical signal each independently has an on time of about 30 seconds to about 30 minutes.

In embodiments, the first electrical signal and the second electrical signal each independently has a current amplitude in a range from about 0.01 mAmps to about 20 mAmps. In embodiments, the first electrical signal and the second electrical signal each independently has a voltage in a range from about 0.01 volts to about 20 volts.

In embodiments, the second electrical signal comprises an abrupt start of pulses, or a ramp up of current/voltage amplitude, or a ramp up of frequency, or a ramping up of pulse widths, or combination thereof at or near initiation of applying the second electrical signal.

In embodiments, the ramp up or ramp down time of current/voltage amplitude, frequency, or pulse widths of the second electrical signal is from about 10 seconds to about 15 minutes.

In embodiments, the ramp up or ramp down of the second electrical signal is linear or non-linear.

In embodiments, the first nerve branch/fiber and the second nerve branch/fiber are independently from a nerve selected from the group consisting of the vagus nerve, renal nerve, renal artery, sympathetic nerves, baroreceptors, glossopharyngeal nerve, a nerve of duodenum, a nerve of jejunum, a nerve of ileum, and combinations thereof.

In embodiments, the first nerve branch/fiber and the second nerve breach/fiber are from different nerves. In embodiments, the first nerve branch/fiber and the second nerve branch/fiber are from the same nerve.

In some embodiments, the present methods and systems relate to treating a subject having a disease or disorder selected from the group consisting of obesity, overweight, pancreatitis, dysmotility, bulimia, gastrointestinal disease with an inflammatory basis, ulcerative colitis, Crohn's disease, low vagal tone, gastroparesis, diabetes, prediabetes, Type II diabetes, chronic pain, hypertension, gastroesophageal reflux disease, peptic ulcer disease and combinations thereof.

In some embodiments, the present disclosure relates to a method for treating a condition associated with impaired glucose regulation of a subject in need thereof comprising applying a first electrical signal to one or more hepatic branch/fiber of a vagus nerve or any segment of the anterior vagus nerve cranial to the branching point of the hepatic branch of the subject and applying a second electrical signal to one or more celiac nerve branch/fiber of the vagus nerve or any segment of the posterior vagus nerve cranial to the branching point of the celiac branch of the subject, wherein the first electrical signal downregulates nerve activity and has a frequency of about 200 Hz to about 100 kHz, and wherein the second electrical signal upregulates nerve activity and has a frequency of about 0.01 Hz to 199 Hz, and wherein the first electrical signal is low duty cycle of about 75% or less. In embodiments, the first electrical signal is a high frequency signal or a high frequency low duty cycle signal according to the present disclosure. In embodiments, the second electrical signal is a low frequency stimulation signal according to the present disclosure. In embodiments, the first electrical signal and the second electrical signal are applied concurrently or simultaneously. In embodiments, the first electrical signal and the second electrical signal are applied at different times. In embodiments, the first electrical signal and the second electrical signal are applied in a coordinated fashion.

In some aspects, the present disclosure relates to a system comprising an implantable neuroregulator; at least one first electrode electrically connected to the implantable neuroregulator and adapted to be placed on a first nerve branch/fiber of a subject; and at least one second electrode electrically connected to the implantable neuroregulator and adapted to be placed on a second nerve branch/fiber of the subject, wherein the implantable neuroregulator comprises a microprocessor, the microprocessor configured to independently deliver a first electrical signal to the first nerve branch/fiber through the first electrode and deliver a second electrical signal to the second nerve branch/fiber through the second electrode, wherein the first electrical signal has parameters to downregulate nerve activity and the second electrical signal has parameters to stimulate nerve activity. In embodiments, the first electrical signal has a frequency of about 200 Hz to about 100 kHz. In embodiments, the second electrical signal has a frequency of about 0.01 Hz to 199 Hz. In embodiments, the first electrical signal is low duty cycle of about 75% or less, or about 50% or less. In embodiments, the microprocessor is configured to independently and concurrently deliver the first electrical signal to the first nerve branch/fiber through the first electrode and deliver the second electrical signal to the second nerve branch/fiber through the second electrode.

In embodiments, the present disclosure relates to a system comprising: an implantable neuroregulator; at least one first electrode electrically connected to the implantable neuroregulator and adapted to be placed on a first nerve branch/fiber of a subject; at least one second electrode electrically connected to the implantable neuroregulator and adapted to be placed on a second nerve branch/fiber of the subject; and at least one third electrode electrically connected to the implantable neuroregulator and adapted to be placed on a third nerve branch/fiber of the subject, wherein the implantable neuroregulator comprises a microprocessor, the microprocessor configured to independently deliver a first electrical signal to the first nerve branch/fiber through the first electrode and deliver a second electrical signal to the second nerve branch/fiber through the second electrode and deliver a third electrical to the third nerve branch/fiber through the third electrode, wherein the first electrical signal downregulates nerve activity and the second electrical signal stimulates nerve activity, and wherein the third electrical signal either downregulates or stimulates nerve activity. In embodiments, the first electrical signal has a frequency of about 200 Hz to about 100 kHz. In embodiments, the second electrical signal has a frequency of about 0.01 Hz to 199 Hz. In embodiments, the first electrical signal is low duty cycle of about 75% or less, or about 50% or less. In embodiments, the microprocessor is configured to independently and concurrently deliver a first electrical signal to the first nerve branch/fiber through the first electrode, deliver the second electrical signal to the second nerve branch/fiber through the second electrode, and deliver the third electrical signal to the third nerve branch/fiber through the third electrode.

In another aspect of the disclosure, the methods of the disclosure can be implemented by a computer, stored as instructions on a microprocessor, stored on an external device such as a mobile phone or charger, or on a computer readable medium.

In other aspects of the disclosure, a system is provided with a microprocessor configured to deliver an electrical signal to a nerve of a subject during an on time, wherein the electrical signal is a high frequency signal or a low frequency stimulation signal according to the present application. In other embodiments, the microprocessor is configured to deliver an electrical signal during an on time that comprises more than one microsecond cycle to form a millisecond active phase, and applying more than one millisecond active phase during the on time, wherein each millisecond active phase is separated by a millisecond inactive phase during the on time. In other embodiments, the microprocessor is configured to deliver an electrical signal to a nerve of a subject during an on time that comprises a first pattern that comprises at least one microsecond cycle; and a second pattern comprising more than one millisecond active phase, wherein each millisecond active phase comprises more than one microsecond cycle, and each millisecond active phase is separated by a millisecond inactive phase. In embodiments, the first and second patterns have different amplitude.

In other embodiments, the microprocessor is configured to independently and respectively deliver multiple electrical signals to multiple nerves or nerve branches/fibers in a subject, wherein the multiple electrical signals include the high frequency signal and low frequency stimulation signal as described in the present disclosure. In embodiments, the microprocessor is configured to concurrently deliver multiple electrical signals to multiple nerves or nerve branches/fibers.

In any embodiment of the methods and systems described herein, the therapy electrical signals can be applied to a nerve or any part thereof including but not limited to a nerve branch, a nerve trunk, a nerve fiber, or any functional segment of a nerve. The therapy signals can also be applied to an organ or any part thereof. Although not exclusively interchangeable, the general description of applying electrical signal(s) to a nerve may also be applied to an organ.

### Definition and Interpretation of Terms

The term “about” is not intended to either expand or limit the degree of equivalents which may otherwise be afforded a particular value. The term “about” in the context of the present disclosure means a value within 10% (±10%) of the value recited immediately after the term “about,” including any numeric value within this range, the value equal to the upper limit (i.e., +10%) and the value equal to the lower limit (i.e., −10%) of this range. For example, the value “100” encompasses any numeric value that is between 90 and 110, including 90 and 110 (with the exception of “100%,” which always has an upper limit of 100%).

“AC” as used herein means alternating current.

“Charge Phase” or “charge and recharge phase” as used herein means a pulse of charge applied to the nerve primarily for high frequency signals. Anodic (positive) phase and cathodic (negative) phase as used herein particularly refer to low frequency stimulation signal.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

“Cycle” as used herein means one repetition of a repetitive pattern of electrical signals. “Stimulation cycle” particularly refers to low frequency stimulation signal.

“Concurrently” used here in generally means that in situations where multiple electrical signals are applied, in at least one time period, the multiple electrical signals are applied simultaneously or about the same time.

“Duty Cycle” as used herein means the percentage of time charge is delivered to the nerve in one cycle. In embodiments, duty cycle can be modified by decreasing pulse width and/or by adding inactive phases between pulses or both.

“Frequency” as used herein means the reciprocal of the period measured in Hertz.

“High Duty Cycle” as used herein refers to a pattern of electrical signals with a duty cycle of about 76% or greater.

“Low Duty Cycle” as used herein refers to a pattern of HFAC/HFAV signals with a duty cycle of about 75% or less.

“High frequency” as used herein generally refers to a frequency of about 200 Hz or more.

“High frequency signal” as used herein generally refers to HFAC or HFAV having a frequency of about 200 Hz or more. High frequency signal is particularly used to downregulate or block nerve activity.

“Low frequency” as used herein generally refers to a frequency of about 200 Hz or less.

“Low frequency signal” or “low frequency stimulation signal” as used herein generally refers to stimulation signal having a frequency of 199 Hz or less. Stimulation signal is particularly used to upregulate or stimulate nerve activity. Certain terminologies are defined and particularly used to describe the low frequency stimulation signal of the present disclosure. Some of the terms used herein are synonymous to other terms that can be found in the related field. Table 1 below shows a few examples of synonymous terms without intent to limit the present disclosure. A person with ordinary skill in the art would be capable of appreciating the terms in light of the definitions thereof, the use thereof in embodiments, and other description provided herein.

TABLE 1
Examples of terms used in the present disclosure synonymous to terms used in references.

| Terms defined and used in | Synonymous terms | |
|---|---|---|
| the present disclosure | used in references | References |
| Stimulation inactive phase | Interpulse interval | Kaczmarek, 1992, Pg. 702; |
| Pulse width | Pulse duration, or width | Merrill, 2005, Pg. 180; |
| Pulse delay | Interphase interval, or | http://www.medistim.com/overview/waveforms.html |
| | interphase delay, or delay | |

“HFAC” as used herein refers to high frequency alternating current.

“HFAV” as used herein refers to high frequency alternating voltage.

“Hz” as used herein refers to Hertz.

“Microsecond cycle” as used herein particularly refers to a high frequency signal.

Microsecond cycle refers to application of an electrical signal in a period comprising at least one charge recharge phase; and a microsecond inactive phase. Optionally, a period includes a pulse delay after the charge phase and/or after the recharge phase.

“Stimulation Cycle” or “Stimulation Active Cycle” as used herein particularly refers to a low frequency stimulation signal. Stimulation Cycle refers to application of a low frequency stimulation signal in a stimulation period comprising at least one pulse; and a stimulation inactive phase. Optionally, a stimulation period includes a pulse delay after the negative phase and/or after the positive phase.

“Microsecond Inactive Phase” as used herein particularly refers to a high frequency signal. Microsecond Inactive Phase means a period of time where no charge is being delivered to the nerve, as measured on a microsecond time scale. A microsecond inactive phase is identified in microseconds.

“Millisecond Active Phase” as used herein particularly refers to a high frequency signal. Millisecond Active Phase means a period of time where two or more microsecond cycles are applied to the nerve.

“Millisecond Cycle” as used herein particularly refers to a high frequency signal. Millisecond Cycle refers to application of an electrical signal that comprises at least two microsecond cycles; and a millisecond inactive phase.

“Stimulation Second Cycle” as used herein particularly refers to a low frequency stimulation signal. Stimulation Second Cycle refers to application of a low frequency stimulation electrical signal that comprises at least two stimulation cycles; and an idle phase.

“Millisecond Inactive Phase” as used herein particularly refers to a high frequency signal. Millisecond Inactive Phase means a period of time wherein no charge is being delivered to the nerve, measured on a millisecond time scale. A millisecond inactive phase is identified in milliseconds.

“Stimulation inactive phase” used herein particularly refers to low frequency stimulation signal. Stimulation inactive phase means a period of time wherein no charge is being delivered to the nerve, measured on a time scale from millisecond to second.

“Idle” or “idle phase” as used herein particularly refers to low frequency stimulation signal. Idle means a period of time wherein no charge is being delivered to the nerve, measured on a minute time scale. An idle phase is identified in minutes.

“Pulsatile Stimulation Waveform” used herein particularly refers to a high frequency signal. Pulsatile Stimulation Waveform refers to application of an electrical signal that comprises at least two stimulation active phases; and an idle phase.

“Off Time” as used herein refers to a period when no charge is being delivered to the nerve. In embodiments, off time is on the order of seconds and/or minutes.

“On Time” refers to a period of time in which multiple micro and/or millisecond cycles and/or stimulation cycle and/or stimulation active phase are applied to the nerve. In embodiments, on time is on the order of seconds and/or minutes.

“Period” refers to the length of time of one charge phase and one recharge phase, which can include one or more pulse delays. “Stimulation period” particularly refers to the length of time of one charge phase and one recharge phase in a low frequency stimulation signal. Stimulation period can also include one or more pulse delays.

“Pulse Amplitude” is the height of the pulse in amperes or voltage relative to the baseline.

“Pulse Delay” as used herein refers to an aspect of the period wherein the impedance across a parallel electrical path with the nerve is at or close to 0 Ohms, with the intention of avoiding any unwanted electrical signals being delivered to the nerve.

“Pulse Width” as used herein refers to the length of time of the pulse.

“Ramp Down” as used herein refers to the period at the end of the application of an electrical signal, or between different patterns of electrical signals, to a nerve of a patient where the pulse amplitude of the signal decreases.

“Ramp Up” as used herein refers to increasing the pulse amplitude until the amplitude desired for therapy is reached at the start of an applied electrical signal or between different patterns of electrical signals. The starting amplitude of ramping may be below the current/voltage threshold of blocking.

“Therapy Cycle” as used herein refers to a discrete period of time that contains one or more on times and off times. The pattern of on and off times within the therapy cycle can be repetitive, non-fixed or randomized throughout a therapy schedule.

“Therapy Parameters” as used herein includes, but is not limited to, frequency, pulse width, pulse amplitude, on time, off time and pattern of electrical signals.

“Therapy Schedule” as used herein refers to the time of day when therapy cycles start, the number of therapy cycles, timing of therapy cycles and duration of the delivery of therapy cycles for at least one day of the week.

“Nerve” used herein generally encompasses a nerve or any part thereof, including but not limited to nerve branch, nerve fiber, trunk, branching point.

“Anterior vagal nerve” or “anterior vagal trunk” distributes fibers on the anterior surface of the esophagus, and consists primarily of fibers from the left vagus. “Posterior vagal nerve” or “posterior vagal trunk” consists primarily of fibers from the right vagus nerve distributed on the posterior surface of the esophagus. Anterior vagal nerve and posterior vagal nerve are two different and separate nerves.

“Hepatic branch” used herein refers to a nerve branch of the anterior vagus nerve below the diaphragm. Hepatic branch encompasses any segment of the anterior vagus nerve cranial to the hepatic branch. In particular, Hepatic branch carries afferent information from the pancreas to the brain and efferent information from the brain to the pancreas.

“Celiac branch” used herein generally refers to a nerve branch of the posterior vagus nerve below the diaphragm. Celiac branch encompasses any segment of the posterior vagus nerve cranial to celiac branch. In particular, celiac branch carries afferent information from the pancreas to the brain and efferent information from the brain to the pancreas.

“Celiac fiber” used herein refers to an afferent or efferent axon that travels within the length of the vagus nerve between the pancreas and the brain. The afferent axon travels from the pancreas through the celiac branch of the vagus nerve where it then travels into the posterior vagus below the level of the diaphragm. The afferent axon next enters the thoracic cavity and primarily into the right cervical segment. The afferent axon then enters the brainstem and form a synaptic connection. The efferent fiber is a part of the parasympathetic nervous system. The preganglionic cell body of the efferent fiber is in the brain stem and travels the length of the vagus nerve (similar to the afferent fiber) to its postganglionic neuron in close proximity to the pancreas.

“Hepatic fiber” used herein refers to an afferent or efferent axon that travels within the length of the vagus nerve between the liver and the brain. The afferent axon travels from the liver through the hepatic branch of the vagus nerve where it then travels into the anterior vagus below the level of the diaphragm. The afferent axon next enters the thoracic cavity and primarily into the left cervical segment. The afferent axon then enters the brainstem and form a synaptic connection. The efferent fiber is a part of the parasympathetic nervous system. The preganglionic cell body of the efferent fiber is in the brain stem and travels the length of the vagus nerve (similar to the afferent fiber) to its postganglionic neuron in close proximity to the liver.

When ranges are provided, the range includes both endpoint numbers as well as all real numbers in between. For example, a range of 200 Hz to 25 kHz includes, for example, 201 to 25 kHz, 202 to 25 kHz, as well as 24,999 Hz to 200 Hz, 24,998 Hz to 200 Hz, and 201 Hz to 24,999 Hz, 202 Hz to 24,998 Hz.

With reference now to the various drawing figures in which identical elements are numbered identically throughout, a description of embodiments of the present disclosure will now be described.

### BRIEF DESCRIPTION OF THE DRAWINGS

FIG. 1A is a schematic representation of a therapy system having features that are examples of inventive aspects of the principles of the present disclosure, the therapy system including a neuroregulator and an external charger.

FIG. 1B is another embodiment of a therapy system having features that are examples of inventive aspects of the principles of the present disclosure.

FIG. 2A is a plan view of an implantable neuroregulator for use in the therapy system of FIG. 1A or FIG. 1B according to aspects of the present disclosure.

FIG. 2B is a plan view of another implantable neuroregulator for use in the therapy system of FIG. 1 a or FIG. 1B according to aspects of the present disclosure.

FIG. 3A is a block diagram of a representative circuit module for the neuroregulator of FIG. 2A and FIG. 2B according to aspects of the present disclosure.

FIG. 3B is a block diagram for a low power arbitrary waveform generator intended for implantable therapeutic devices. Some of the functionality is optional such as the memory and telemetry blocks.

FIG. 4 is a block diagram of a circuit module for an external charger for use in the therapy system of FIG. 7A or FIG. 1B according to aspects of the present disclosure.

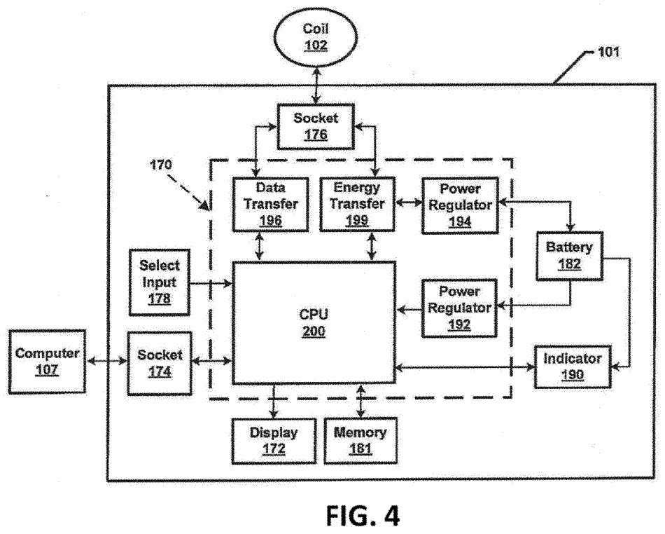

FIG. 5 is a representation of a prior art waveform displaying a high duty cycle HFAC/HFAV signal. In this example the period is 5 milliseconds or less making the frequency 200 Hz or greater which is considered a high frequency signal.

FIG. 6 is a representation of a pattern of electrical signals displaying a repetitive low duty cycle HFAC/HFAV on the microsecond scale wherein the charge/recharge phases are followed by a substantially longer microsecond inactive phase to form a microsecond cycle. The microsecond cycle includes more than one period. This pattern repeats itself for the duration of an on time.

FIG. 7 is a representation of a pattern of electrical signals displaying a low duty cycle HFAC/HFAV which contains microsecond cycles. Within the microsecond cycles are charge and recharge phases separated by pulse delays and microsecond inactive phases following the pulse delay that follows the recharge phase. The charge recharge phase and the pulse delays form a period. The pulse delay between the charge and recharge phase is equal in time to the pulse delay following the recharge phase.

FIG. 8 is a representation of a layered pattern of electrical signals displaying a low duty cycle HFAC/HFAV algorithm which contains microsecond cycles, with long microsecond inactive phases, which are repeated to form a millisecond active phase which is followed by a millisecond inactive phase. This pattern repeats itself for the duration of an on time.

FIG. 9 is a representation of a pattern of layered electrical signals displaying a low duty cycle HFAC/HFAV which contains repetitive microsecond cycles which form a millisecond active phase. Within the microsecond cycles are charge and recharge phases separated by pulse delays and microsecond inactive phases following the pulse delay that follows the recharge phase. The pulse delay between the charge and recharge phase is equal in time to the pulse delay following the recharge phase.

FIG. 10 is a representation of a layered pattern of electrical signals displaying a HFAC/HFAV low duty cycle with microsecond cycles that contain a charge and recharge phase followed by a long microsecond inactive phase. The microsecond cycles are repeated at first pulse amplitude for a period of time (on the order of seconds). Following this (on the order of seconds) the pulse amplitude is decreased to a second pulse amplitude and the repeated microsecond cycles form millisecond active phases. Each millisecond active phase is followed by a millisecond inactive phase at the second amplitude.

FIG. 11 is a representation of a layered pattern of electrical signals displaying a HFAC/HFAV low duty cycle with microsecond cycles that contain a charge and recharge phase followed by a long microsecond inactive phase. The microsecond cycles are repeated at first pulse amplitude for a period of time (on the order of seconds). Following this (on the order of seconds) the pulse amplitude is decreased to a second amplitude using a ramp down and the repeated microsecond cycles form millisecond active phases. Each millisecond active phase is followed by a millisecond inactive phase at the second amplitude.

FIG. 12 illustrates an exemplary architecture of a computing device that can be used to implement aspects of the present disclosure.

FIG. 13 is a flowchart illustrating an exemplary method of operating the therapy system.

FIG. 14 illustrates a plurality of parameters usable for various types of therapy treatment signals.

FIG. 15 is a flowchart illustrating an exemplary method for operation the therapy system for a first therapy program for regulating nerve activity.

FIG. 16 is a flowchart illustrating an example method 470 for operation the therapy system for a second therapy program for regulating nerve activity.

FIG. 17 is a flowchart illustrating an example method 490 for operation the therapy system for a third therapy program for regulating nerve activity.

FIG. 18 is a representation of a pulse width of 10 microseconds at a frequency of 10,000 Hz with no pulse ramp down. At a pulse width of 10 microseconds, repetitive firing and tetany is observed, and no block of nerve conduction is seen. This profile represents a pulse width at or below a lower boundary threshold.

FIG. 19 is a representation of an example of ramping down pulse width to a pulse width below the boundary threshold. At a frequency of 10,000 Hz and an initial pulse width of 30 microseconds (60% duty cycle) the pulse width is decreased to 25 microseconds for 20 seconds. Next the pulse width would decrease to 20 microseconds for 20 seconds and next to 15 microseconds for 20 seconds and follow the same pattern until the pulse width reaches 5 microseconds (10% duty cycle) and is constant for the duration of the on time. Blocking of the nerve occurs at pulse width of 5 microseconds at a frequency of 10,000 Hz with a current amplitude of 0.1 mA to 20 mA.

FIG. 20 is a representation of an exemplary embodiment of a pulse width ramp down with time between pulses decreasing.

FIG. 21 is a representation of an exemplary embodiment of a pulse width ramp up with time between pulses decreasing.

FIG. 22 is a representation of an exemplary embodiment of a pulse width ramp down in combination with current/voltage ramp down and no pulse delays.

FIG. 23 is a representation of an exemplary embodiment of a pulse width ramp down in combination with current/voltage ramp down and pulse delays.

FIG. 24 is a representation of an exemplary embodiment of a pulse width ramp down in combination with current/voltage ramp down and no pulse delays in 2 cycle steps.

FIG. 25 is a representation of an exemplary embodiment of a pulse width and current (or voltage) ramp down followed by a steady state low duty cycle signal and a pulse width and current (or voltage) ramp up before termination of the signal.

FIG. 26 is a representation of an exemplary embodiment of a steady state low duty cycle followed by a ramp up of pulse width and current (or voltage) before termination of the signal.

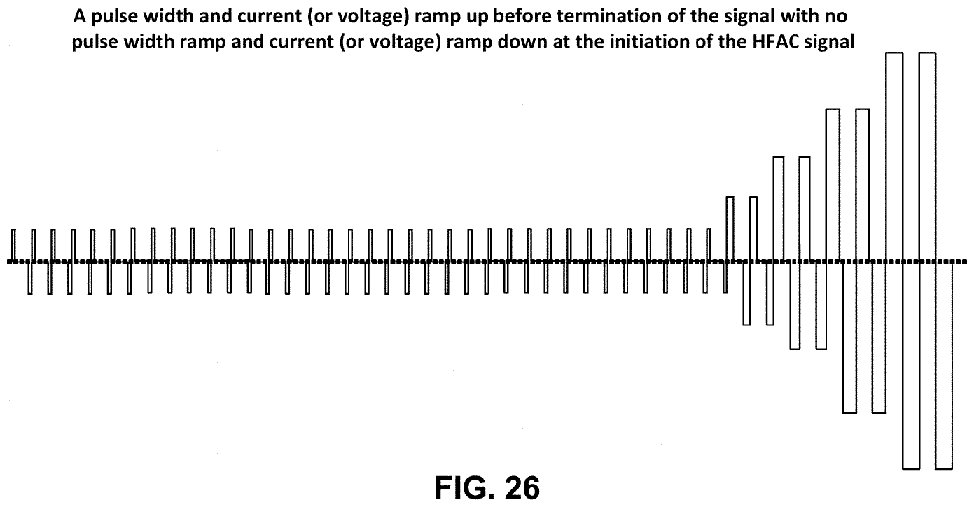

FIG. 27(a) represents an exemplary embodiment of a biphasic pulse; FIG. 27(b) represents an exemplary embodiment of a monophasic pulse.

FIG. 28 is a representation of a pattern of upregulating electrical signals displaying a repetitive low frequency stimulation signal comprising one or more stimulation periods, each stimulation period comprising one or more pulses and optionally a stimulation inactive phase.

FIG. 29 represents a continuous low frequency stimulation signal waveform comprising more than one stimulation cycle without an idle phase.

FIG. 30 represents a pulsatile low frequency stimulation signal waveform comprising more than one stimulation active phase, each stimulation active phase separated by an idle phase.

FIG. 31 represents an exemplary embodiment of a ramp up/down of amplitude/voltage for low frequency stimulation signal, with the amplitude/voltage between pulses increasing. (Ramp down having the amplitude/voltage decrement of pulses decreasing is not explicitly shown but can be appreciated by a skilled artisan).

FIG. 32 represents an exemplary embodiment of frequency ramp up/down for low frequency stimulation signal, with the frequency of pulses increasing. (Ramp down signal having the frequency decrement of pulses decreasing is not explicitly shown but can be appreciated by a skilled artisan).

FIG. 33 represents an exemplary embodiment of pulse width ramp up/down for low frequency stimulation signal, with the pulse width of pulses increasing. (Ramp down signal having the pulse width decrement of pulses decreasing is not explicitly shown but can be appreciated by a skilled artisan).

FIG. 34(a) shows a diagram of nerve/electrode used in Examples 2 and 3. *SIF soaked gauze used to control impedance between blocking electrodes. Sd=distal stimulation electrode, Sp=proximal (control) stimulation electrode and R=recording electrode; FIG. 34(b) shows the HFAC signal used in Example 2 and 3. The HFAC signal consists of a charge balanced 5,000 Hz alternating current square waveforms (90 μs pulse width) with pulse delays (10 μs) between the charge and recharge phases. During the pulse delays the electrodes were short-circuited to eliminate DC offset on the nerve.

FIG. 35 shows the effect of HFAC amplitude on the degree of nerve conduction block in Example 2. The traces from top to bottom are CAPs evoked immediately following the application of 60 seconds HFAC at current amplitudes of 0, 3, 5, 8 and 10 mA. The faster Aδ wave has a peak CV of 9.4 m s⁻¹. The slower C wave has a peak CV of 0.85 m s⁻¹.

FIG. 36 shows Blockade of Aδ (a) and C waves (b) of CAPs evoked immediately following the delivery of different HFAC amplitudes at various HFAC durations in Example 2. CAP amplitudes were normalized to baseline.

FIG. 37 shows the recovery times of Aδ (a) and C waves (b) following the delivery of different HFAC amplitudes at various HFAC durations in Example 2. For the Aδ wave, recovery time was significantly influenced by both the HFAC duration and amplitude. For the C wave, recovery time was significantly influenced by the HFAC amplitude, and there was a substantial leftward shift in the current-effect curve for 120 second HFAC duration compared to a 60 second HFAC duration at current amplitudes above 5 mA.

FIG. 38 shows the time course of recovery following cessation of HFAC in Example 2: (a) A plot of the average Aδ wave amplitude versus time following the delivery of HFAC with a 5 mA amplitude at 60 and 120 seconds of HFAC duration; (b) A plot of the average C wave amplitude versus time following termination of HFAC at an HFAC amplitude of 10 mA for 60 and 120 seconds of HFAC duration; (c) A plot of the average Aδ wave amplitude versus time following the cessation of HFAC with 30 seconds at 10 mA HFAC compared with 120 seconds at 5 mA HFAC.

FIG. 39 shows the degree of sustained block during recovery of Aδ (a) and C waves (b) following the delivery of different HFAC amplitudes at various HFAC durations in Example 2. For the Aδ and C waves, both HFAC duration and amplitude significantly influenced the amount of block during recovery.

FIG. 40 shows average Aδ wave amplitudes elicited by a proximal and distal stimulating electrode during and following 5,000 Hz at a HFAC duration of 120 seconds (a) and 30 seconds (b) with a 5 mA current amplitude in Example 2. The CAP generated by the proximal electrode was not considerably depressed compared to the CAP elicited by the distal electrode during and following 5000 Hz for both conditions.

FIG. 41 shows average C wave amplitudes elicited by a proximal and distal stimulating electrode during and following 5,000 Hz at a HFAC duration of 120 seconds at 10 mA (a) and 8 mA second (b). The CAP generated by the proximal electrode was not considerably depressed compared to the CAP elicited by the distal electrode during and following 5,000 Hz for a full (a) or partial (b) distal CAP attenuation.

FIG. 42(a) shows the effect of HFAC amplitude on the degree of nerve conduction block in Example 3. FIG. 42(b) shows average Aδ wave amplitudes elicited by a proximal and distal stimulating electrode during and following 5000 Hz at a HFAC duration of 120 seconds with a 5 mA current amplitude in Example 3. FIG. 42(c) shows average Aδ wave amplitudes elicited by a proximal and distal stimulating electrode during and following 5000 Hz at a HFAC duration of 30 seconds with a 5 mA current amplitude in Example 3.

FIG. 43(a) shows the degree of HFAC-induced nerve blockage in Example 3. FIG. 43(b) shows the recovery of the HFAC-induced nerve blockage in Example 3.

FIG. 44(a) shows High duty cycle 5000 Hz signal used in Example 3. During 10 μs pulse delays between the charge and recharge phase the electrodes were short circuited to dissipate any direct current offset on the nerve. FIG. 44(b) shows a 1000 Hz signal used in Example 3. The signal has 90 μs pulse widths incorporated 820 μs inactive periods to decrease the duty cycle by about 5 fold. FIG. 44(c) shows a 1000 Hz signal used in Example 3. The signal has microsecond inactive periods was interwoven with 20 millisecond inactive periods which decreased the duty cycle by 1 order of magnitude.

FIG. 45(a) shows a diagram of carry over block with the low duty cycle signals with similar recovery kinetics as the high duty cycle (5000 Hz) signal. Signal was applied for 1 min at 5 mAmp. FIG. 45(b) shows a diagram of similar current-effect curve with the high duty cycle (5000 H)z signal and the low duty cycle signals of Example 3.

FIG. 46 shows the results of strength duration for sub-diaphragmatic pig vagus nerve stimulation in Example 4. The lower the curve the lower energy is needed for stimulation.

FIG. 47 illustrates plasma glucose (PG) measurement results of Example 5. HFAC was applied to the hepatic branch of the vagus nerve with simultaneous stimulation of the celiac branch of the vagus nerve reversibly following an IVGTT in control and ZDF rats.

FIG. 47(a) shows a positive control the hepatic nerve was ligated (in place of application of HFAC) with concurrent stimulation of the celiac nerve; FIG. 47(b) shows the plasma glucose results from the application of HFAC/stimulation following the IVGTT compared to sham; FIG. 47(c) shows the plasma glucose results of HFAC/stimulation and vagotomy/stimulation applied to Control Sprague Dawley rats.

FIG. 48 shows an example of alloxan treated and control Yucatan pig, which wore a specially designed jacket to house two of the ReShape Lifesciences Inc. Maestro® mobile chargers in Example 6. Transmit coils above the layer of the skin were connected to the mobile chargers to charge the implanted neuroregulators with a RF signal between experiments. Settings for stimulation and HFAC parameters were also programmed into the neuroregulators using a laptop computer and application software via the mobile chargers and transmit coils. Pigs were trained for 7 days prior to pre-implant OGTTs to wear the jacket. During the charging sessions the pigs were not restrained and there was no apparent stress to the animals.

FIG. 49 demonstrates conduction block and recovery at a HFAC amplitude of 8 mA as resulted from the isolated pig sub-diaphragmatic vagus nerve electrophysiology in Example 6. FIG. 49(a) shows representative CAP elicited by electrical stimulation at the level below the diaphragm and recorded at a segment below the heart (Scale bar: 35 ms×200 μV). The length of the isolated vagus nerve was approximately 35 mm; FIG. 49(b) shows the current-effect curve of CAP amplitude elicited from the proximal and distal stimulation electrodes immediately following the cessation of HFAC; FIG. 49(c) shows the diagram of recovery of CAP amplitude following full block.

FIG. 50 shows the ReShape Lifesciences Inc. Maestro© electrodes which were used in the in Example 5 and 6. Two of these electrodes were sutured to the esophagus next to each other (approximately 4 mm separation between contacts) and cradled the nerve.

FIG. 51(a) shows plasma glucose levels following an IVGTT prior to and following the administration of Alloxan in Example 4; FIG. 51(b) shows plasma glucose levels following an OGTT in Alloxan treated pigs and control non-Alloxan treated pigs in Example 6.

FIG. 52 illustrate the results of application of HFAC to the hepatic fiber of the vagus nerve with simultaneous stimulation of the celiac fiber of the vagus nerve in Alloxan treated pigs in Example 4. FIG. 52(a) shows the results from an OGTT conducted prior to implant of the device and following the implant of device; FIG. 52(b) shows the results from application of HFAC/stimulation 2 hours prior to or 5 minutes following the initiation of the OGTT significantly in Alloxan treated pigs; FIG. 52(c) shows results following HFAC/stimulation applications an OGTT with the devices off.

FIG. 53 shows the blood glucose change over time in diabetic swine treated with the combination of blocking the hepatic vagus nerve fiber and stimulating the celiac vagus nerve fiber in Example 7. Initiation of BLK/STIM treatment was performed 5 min following glucose administration.

FIG. 54 shows blood glucose change over time in diabetic swine treated with the combination of blocking (BLK) the hepatic vagus nerve fiber and stimulating (STIM) the celiac vagus nerve fiber in Example 7. Initiation of BLK/STIM treatment was performed 30 min following glucose administration.

FIG. 55 shows blood glucose change over time in diabetic swine treated with the combination of blocking the hepatic vagus nerve fiber and stimulating the celiac vagus nerve fiber in Example 7. Initiation of BLK/STIM treatment was performed 5 min following glucose administration. The blocking signal is a HFAC low duty cycle signal having an intermittent blocking pattern with 990 milliseconds on and 10 milliseconds off.

### DETAILED DESCRIPTION

The particulars shown herein are by way of example and for purposes of illustrative discussion of the invention. In this regard, no attempt is made to show structural details of the invention in more detail than is necessary for the fundamental understanding of the invention, the description taken with the drawings making apparent to those skilled in the art how the several forms of the invention may be embodied in practice.

A. Therapy System

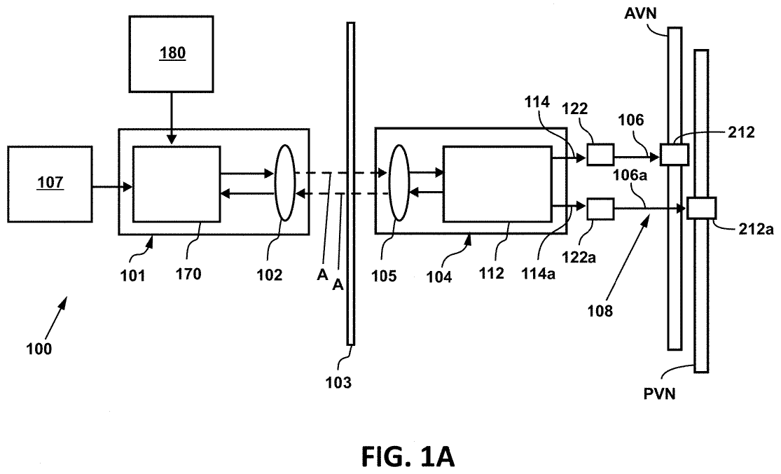

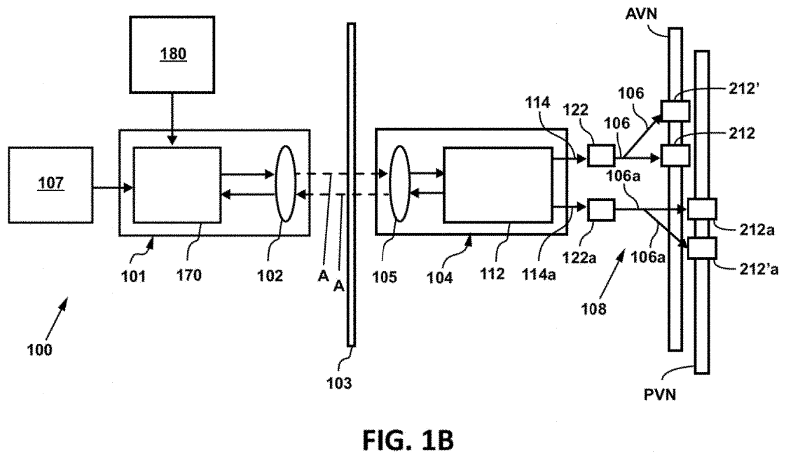

FIG. 1A schematically illustrates a therapy system 100. The therapy system 100 includes a neuroregulator 104, an electrical lead arrangement 108, and an external charger 101. The neuroregulator 104 is adapted for implantation within a subject. As will be more fully described herein, the neuroregulator 104 typically is implanted just beneath a skin layer 103.

The neuroregulator 104 is configured to connect electrically to the lead arrangement 108. In general, the lead arrangement 108 includes two or more (first and second) electrical lead assemblies, 106, 106a. In embodiments, a single lead comprises at least two electrodes. In other embodiments, each lead comprises a single electrode. In the example shown, the lead arrangement 108 includes first and second (bipolar) electrical lead assemblies 106, 106a. The neuroregulator 104 generates therapy electrical signals and transmits the therapy electrical signals to the first and second lead assemblies 106, 106a. The first and second lead assemblies 106, 106a stimulate (upregulate the nerve activity) and/or block (downregulate the nerve activity) conduction of nerves of a subject based on the therapy electrical signals provided by the neuroregulator 104. In an embodiment, first and second lead assemblies 106, 106a include first and second distal electrodes, 212, 212a, which are placed on one or more nerves or nerve branches/fibers of a patient. For example, the first electrodes 212, 212a may be individually placed on the anterior vagal nerve AVN and posterior vagal nerve PVN, respectively, of a patient. In an embodiment, first and second lead assemblies 212, 212a can be placed on the vagal nerve at a subdiaphragmatic location. For example, the distal electrodes 212, 212a can be placed just below the patient's diaphragm. In other embodiments, however, fewer or more electrodes can be placed on or near fewer or more nerves.

In some embodiments, the electrical lead assemblies and electrodes can be configured to deliver a signal having biphasic pulses (e.g., a pulse having a positive phase/charge followed by a negative phase/charge, or alternatively a pulse having a negative phase followed by a positive phase). In other embodiments, the electrical lead assemblies and electrodes can be configured to deliver a signal having monophasic pulse (e.g. a positive phase/charge, or a negative phase/charge).

FIG. 1B shows another embodiment of the present therapy system comprising one neuroregulator 104 and lead assemblies 106, 106a electrically connected to the neuroregulator. Lead assembly 106 connects to two distal electrodes 212 and 212′, both individually placed on one nerve or nerve branch/fiber (e.g., AVN). Lead assembly 106a connects to two distal electrodes 212a and 212a′, both individually placed on a separate nerve or separate nerve branch/fiber (e.g., PVN). The neuroregulator 104 is configured to independently and separately deliver a first therapy electrical signal through electrodes 212 and 212′, and deliver a second therapy electrical signal through electrodes 212a and 212a′. The first and second therapy signals can independently be a high frequency signal, a high frequency low duty cycle signal, or a low frequency stimulation signal, or other signals according to the present disclosure.

The external charger 101 includes circuitry for communicating with the implanted neuroregulator 104. In general, communication is transmitted across the skin 103 along a two-way signal path as indicated by arrows A. Example communication signals transmitted between the external charger 101 and the neuroregulator 104 include therapy instructions, patient data, and other signals as will be described herein. Energy also can be transmitted from the external charger 101 to the neuroregulator 104 as will be described herein.

In the example shown, the external charger 101 can communicate with the implanted neuroregulator 104 via bidirectional telemetry (e.g. via radio frequency (RF) signals). The external charger 101 shown in FIG. 1A includes an external coil 102, which can send and receive RF signals. A similar internal coil 105 can be implanted within the patient and electrical communication with the neuroregulator 104. In an embodiment, the internal coil 105 is integral with the neuroregulator 104. The internal coil 105 serves to receive and transmit signals from and to the coil 102 of the external charger 101.

For example, the external charger 101 can encode the information as a bit stream by amplitude modulation or frequency modulation of an RF carrier wave. The signals transmitted between the external and internal coils 102, 105 preferably have a carrier frequency of about 6.78 MHz. For example, during an information communication phase, the value of a parameter can be transmitted by toggling a rectification level between half-wave rectification and no rectification. In other embodiments, however, higher or lower carrier wave frequencies may be used.

In an embodiment, the neuroregulator 104 communicates with the external charger 101 using load shifting (e.g., modification of the load induced on the external charger 101). This change in the load can be sensed by the inductively coupled external charger 101. In other embodiments, however, the neuroregulator 104 and external charger 101 can communicate using other types of signals.

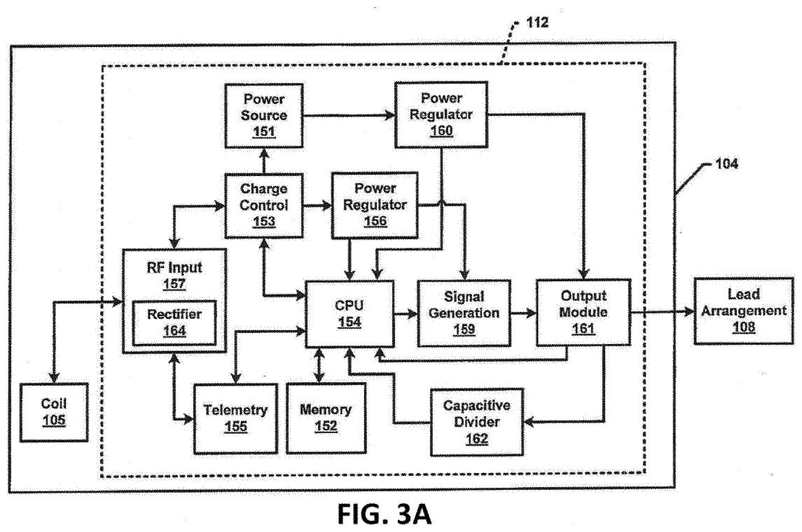

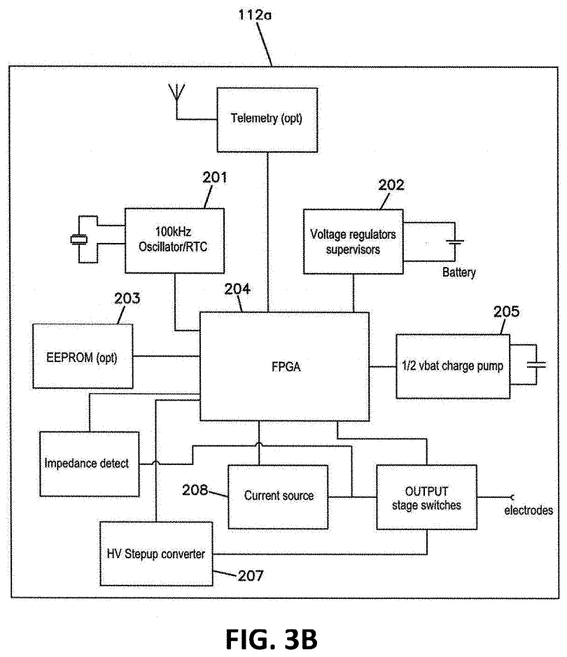

In an embodiment, the neuroregulator 104 receives power to generate the therapy signals from an implantable power source 151 (see FIG. 3A), such as a battery. In a preferred embodiment, the power source 151 is a rechargeable battery. In some embodiments, the power source 151 can provide power to the implanted neuroregulator 104 when the external charger 101 is not connected. In other embodiments, the external charger 101 also can be configured to provide for periodic recharging of the internal power source 151 of the neuroregulator 104. In an alternative embodiment, however, the neuroregulator 104 can entirely depend upon power received from an external source. For example, the external charger 101 can transmit power to the neuroregulator 104 via an RF link (e.g., between coils 102, 105).

In another embodiment, the neuroregulator comprises a non-rechargeable primary cell battery (not shown in figures). This system would not have to be recharged and is meant to work on the order of years before a replacement neuroregulator is implanted.

In embodiments, the neuroregulator 104 can be powered by a rechargeable battery 151, which is periodically charged by the application of the mobile charger 101, the latter being placed in close proximity to the implanted neuroregulator 104. Alternatively, the neuroregulator 104 can be directly powered by RF energy provided by the mobile charger 101. The choice of the mode of providing power is made via a setting of the mobile charger 101, or via the clinician programmer. In a further embodiment, charging of the rechargeable battery 151 in the neuroregulator 104, can be achieved by application of remote wireless energy. (Grajski et al, IEEE Microwave Workshop series on Innovative Wireless Power Transmission: Technology, Systems, and Applications, 2012 published on a4wp.org).

In some embodiments, the neuroregulator 104 initiates the generation and transmission of therapy electrical signals to the first and second lead assemblies 106, 106a. In an embodiment, the neuroregulator 104 initiates therapy when powered by the internal battery 151. In other embodiments, however, the external charger 101 triggers the neuroregulator 104 to begin generating therapy electrical signals. After receiving initiation signals from the external charger 101, the neuroregulator 104 generates the therapy signals and transmits the therapy signals to the first and second lead assemblies 106, 106a.

In embodiments, the neuroregulator comprises a microprocessor (e.g. FIG. 3A; 154). In embodiments, the microprocessor is configured to deliver a high frequency signal or high frequency low duty cycle signal according to the present disclosure. In embodiments, the microprocessor is configured to deliver a low frequency stimulation signal according to the present disclosure.

In embodiments, a microprocessor is configured to deliver an electrical signal to a nerve of a subject during an on time that comprises more than one microsecond cycle comprising more than one period, each period comprising a charge recharge phase which may or may not have pulse delays, each period having a frequency of about at least 200 Hz; and a microsecond inactive phase.

In other embodiments, the microprocessor is configured to deliver an electrical signal to a nerve of a subject during an on time that comprises more than one microsecond cycle to form a millisecond active phase, and applying more than one millisecond active phase during the on time, wherein each millisecond active phase is separated by a millisecond inactive phase during the on time. In embodiments, a microsecond cycle comprises at least one period comprising a charge recharge phase, and optionally, a pulse delay, each period having a frequency of about at least 200 Hz; and a microsecond inactive phase.

In other embodiments, the microprocessor is configured to deliver an electrical signal to a nerve of a subject during an on time that comprises a first pattern comprising at least one microsecond cycle; and a second pattern comprising more than one millisecond active phase, wherein each millisecond active phase comprises more than one microsecond cycle, and each millisecond active phase is separated by a millisecond inactive phase. In embodiments, the first and second patterns have a different amplitude. In embodiments, the first pattern and second pattern are separated by a ramp up and/or ramp down in amplitude.

In some embodiments, the microprocessor is configured to deliver a low frequency electrical signal to a nerve or a nerve branch/fiber or an organ, wherein the low frequency electrical signal is in a range from about 0.01 Hz to about 100 Hz, preferably from about 0.01 Hz to about 30 Hz. In embodiments, the stimulation signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic recharge phase and optionally a pulse delay, the pulse having a pulse width. In embodiments, the stimulation signal further comprises at least one stimulation active phase, wherein each of the at least one stimulation active phase comprises at least one stimulation active cycle, and wherein each of the at least one stimulation active phase is separated by an idle.

In some embodiments, the microprocessor is configured to independently and respectively deliver multiple electrical signals to multiple nerves or nerve branches/fibers, wherein each of the electrical signals have parameters that either downregulate or upregulate the nerve activity of the nerve or nerve branches/fibers where the corresponding electrical signal is applied to. In embodiments, the microprocessor is configured to concurrently or simultaneously deliver multiple electrical signals to multiple nerves or nerve branches/fibers. In embodiments, the microprocessor is configured to deliver multiple electrical signals to multiple nerves or nerve branches/fibers in a coordinated fashion.

In some embodiments, the microprocessor is configured to independently and separately deliver a first electrical signal to a first nerve and deliver a second electrical signal to a second nerve, wherein the first electrical signal downregulates nerve activity and has a frequency from about 200 Hz to about 100 kHz, and wherein the second electrical signal upregulates nerve activity and has a frequency from about 0.01 Hz to 199 Hz. In embodiments, the first electrical signal and the second electrical signal are applied concurrently or simultaneously. In embodiments, the first electrical signal comprises at least one microsecond cycle and optionally a microsecond inactive phase, wherein each of the at least one microsecond cycle comprises at least one period, each of the at least one period comprising a pulse comprising a charge recharge phase, the pulse having a pulse width, and wherein the second electrical signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width.

In other embodiments, the external charger 101 also can provide the instructions according to which the therapy signals are generated (e.g., frequency, pulse-width, amplitude, and other such parameters). In a preferred embodiment, the external charger 101 includes memory 203 in which individual parameters, programs, and/or therapy schedules can be stored for transmission to the neuroregulator 104. In embodiments, those parameters include parameters for the high frequency signals such as frequency, the time of a microsecond inactive phase, the time of a millisecond active phase, and/or the time of a millisecond inactive phase, and the parameters of the low frequency stimulation signal such as frequency, the time of a stimulation active phase, the time of a stimulation inactive phase, the time of an idle phase, at son on. In alternative embodiment, the parameters include frequency, the % of duty cycle of the microsecond cycle and/or the % of duty cycle of the millisecond cycle.

Selection of those parameters can be made by a user on a user interface (not shown). In embodiments, those parameters include pulse width, constant voltage settings, constant current settings, frequency, % duty cycle of the microsecond cycle and/or millisecond cycle, amplitude, microsecond inactive phase time, millisecond active phase time, and/or a millisecond inactive phase, and/or stimulation cycle, stimulation active phase, stimulation inactive phase, idle phase, etc. The external charger 101 also can enable a user to select a parameter/program/therapy schedule as displayed on a user interface, and then be stored in memory for transmission to the neuroregulator 104. As disclosed herein each of the methods can form a therapy program. In another embodiment, the external charger 101 can provide therapy instructions with each initiation signal.

Typically, each of the parameters/programs/therapy schedules stored on the external charger 101 can be adjusted by a practioner to suit the individual needs of the subject. For example, a computing device (e.g., a notebook computer, a personal computer, etc.) 107 can be communicatively connected to the external charger 101. With such a connection established, a physician can use the computing device 107 to program parameters and/or therapies into the external charger 101 for either storage or transmission to the neuroregulator 104.

The neuroregulator 104 also may include memory 152 (see FIG. 3A) in which therapy instructions and/or subject data can be stored. For example, the neuroregulator 104 can store therapy programs or individual parameters indicating what therapy should be delivered to the subject. The neuroregulator 104 also can store patient data indicating how the subject utilized the therapy system 100 and/or reacted to the delivered therapy.

In some aspects, the present disclosure relates to a system comprising: at least one electrode adapted to be placed on a nerve in a subject; an implantable neuroregulator comprising a rechargeable battery, and microprocessor configured to deliver a high frequency signal having a frequency of at least 200 Hz. In embodiments, the high frequency signal comprises more than one microsecond cycle to form a millisecond active phase. In embodiments, the electrical signal further comprises more than one millisecond active phase during the on time, wherein each millisecond active phase is separated by a millisecond inactive phase during the on time, wherein the microsecond cycle comprises at least one period, each period comprising a charge recharge phase and optionally, at least one pulse delay, each period having a frequency of at least 1000 Hz; and a microsecond inactive phase.

In some aspects, the present disclosure relates to a system comprising: at least one electrode adapted to be placed on a nerve in a subject; an implantable neuroregulator comprising a rechargeable battery, and microprocessor configured to deliver a low frequency stimulation signal having a frequency of at most 199 Hz. In embodiments, the stimulation signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. In embodiments, the stimulation signal further comprises at least one stimulation active phase, wherein each of the at least one stimulation active phase comprises at least one stimulation active cycle, and wherein each of the at least one stimulation active phase is separated by an idle.

In some aspects, the present disclosure relates to a system comprising an implantable neuroregulator; at least one first electrode electrically connected to the implantable neuroregulator and adapted to be placed on a first nerve of a subject; and at least one second electrode electrically connected to the implantable neuroregulator and adapted to be placed on a second nerve of the subject, wherein the implantable neuroregulator comprises a rechargeable battery and a microprocessor, the microprocessor configured to independently deliver a first electrical signal to the first nerve through the first electrode and deliver a second electrical signal to the second nerve through the second electrode, wherein the first electrical signal has parameters to downregulate nerve activity and the second electrical signal has parameters to stimulate nerve activity. In embodiments, the first electrical signal has a frequency of about 200 Hz to about 100 kHz. In embodiments, the second electrical signal has a frequency of about 0.01 Hz to 199 Hz. In embodiments, the first electrical signal is low duty cycle of about 75% or less, or about 50% or less. In embodiments, the microprocessor is configured to independently and concurrently deliver the first electrical signal to the first nerve through the first electrode and deliver the second electrical signal to the second nerve through the second electrode.

In embodiments, the present disclosure relates to a system comprising: an implantable neuroregulator; at least one first electrode electrically connected to the implantable neuroregulator and adapted to be placed on a first nerve of a subject; at least one second electrode electrically connected to the implantable neuroregulator and adapted to be placed on a second nerve of the subject; and at least one third electrode electrically connected to the implantable neuroregulator and adapted to be placed on a third nerve of the subject, wherein the implantable neuroregulator comprises a microprocessor, the microprocessor configured to independently deliver a first electrical signal to the first nerve through the first electrode and deliver a second electrical signal to the second nerve through the second electrode and deliver a third electrical to the third nerve through the third electrode, wherein the first electrical signal downregulates nerve activity and the second electrical signal stimulates nerve activity, and wherein the third electrical signal either downregulates or stimulates nerve activity. In embodiments, the first electrical signal has a frequency of about 200 Hz to about 100 kHz. In embodiments, the second electrical signal has a frequency of about 0.01 Hz to 199 Hz. In embodiments, the first electrical signal is low duty cycle of about 75% or less, or about 50% or less. In embodiments, the microprocessor is configured to independently and concurrently deliver a first electrical signal to the first nerve through the first electrode, deliver the second electrical signal to the second nerve through the second electrode, and deliver the third electrical signal to the third nerve through the third electrode.

1. System Hardware Components

a. Neuroregulator

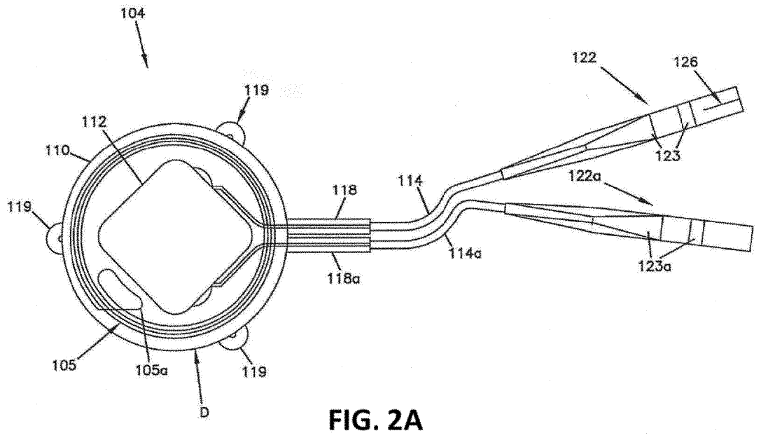

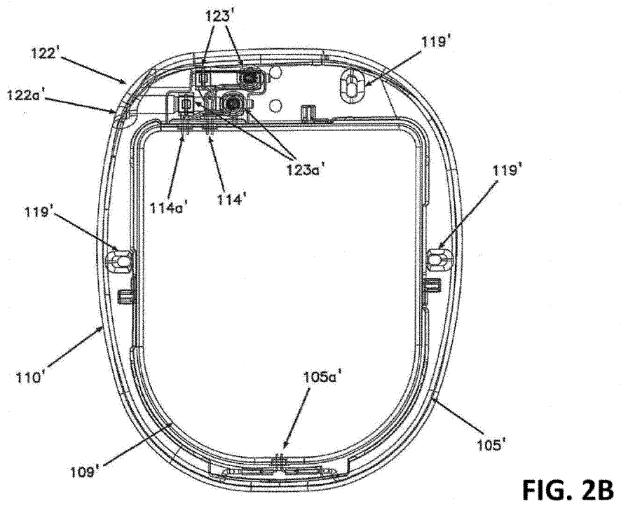

Different embodiments of the neuroregulator 104, 104′ are illustrated schematically in FIGS. 2A and 2B, respectively. The neuroregulator 104, 104′ is configured to be implanted subcutaneously within the body of a subject. In embodiments, the neuroregulator 104, 104′ is implanted subcutaneously on the thoracic sidewall in the area slightly anterior to the axial line and caudal to the arm pit. In other embodiments, alternative implantation locations may be determined by the implanting surgeon.

Typically, the neuroregulator 104, 104′ is implanted parallel to the skin surface 103 to maximize RF coupling efficiency with the external charger 101. In an embodiment, to facilitate optimal information and power transfer between the internal coil 105, 105′ of the neuroregulator 104, 104′ and the external coil 102 of the external charger 101, the patient can ascertain the position of the neuroregulator 104, 104′ (e.g., through palpation or with the help of a fixed marking on the skin). In an embodiment, the external charger 101 can facilitate coil positioning.

As shown in FIGS. 2A and 2B, the neuroregulator 104, 104′ generally includes a housing 109, 109′ overmolded with the internal coil 105, 105′, respectively. The overmold 110, 110′ of the neuroregulator 104, 104′ is formed from a bio-compatible material that is transmissive to RF signals (i.e., or other such communication signals). Some such bio-compatible materials are known in the art. For example, the overmold 110, 110′ of the neuroregulator 104, 104′ may be formed from silicone rubber or other suitable materials. The overmold 110, 110′ can also include suture tabs or holes 119, 119′ to facilitate placement within the patient's body.

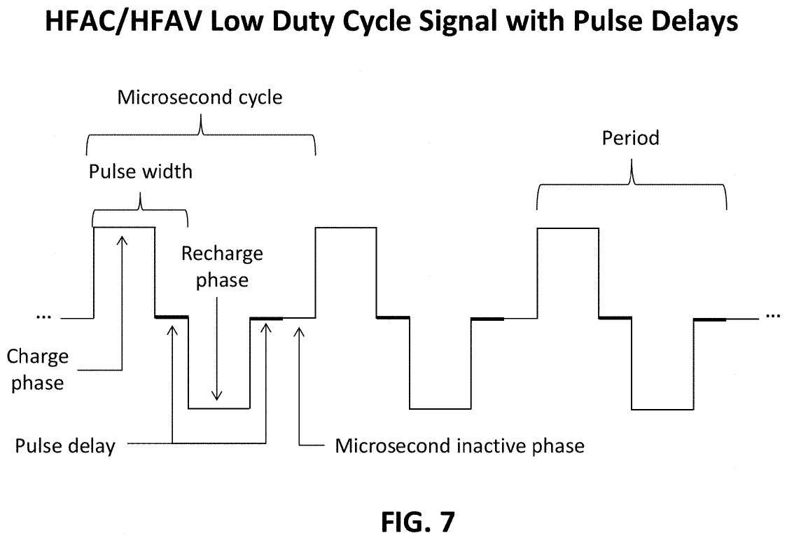

The housing 109, 109′ of the neuroregulator 104, 104′ also may contain a circuit module, such as circuit 112 (see FIGS. 1, 3A, and 3B), to which the coil 105, 105′ may be electrically connected along a path 105a, 105a′. The circuit module within the housing 109 may be electrically connected to a lead assembly, for example, the lead assemblies 106, 106a (FIG. 7A or FIG. 1B) through first and second conductors 114, 114a. In other embodiments, a single lead may be employed. In the example shown in FIG. 2A, first and second conductors 114, 114a extend out of the housing 109, 109′ through first and second strain reliefs 118, 118a. Such conductors 114, 114a are well known in the art.

The conductors 114, 114a terminate at first and second connectors 122, 122a, which are configured to receive or otherwise connect the lead assemblies 106, 106a (FIG. 1A or FIG. 1B) to the conductors 114, 114a. By providing connectors 122, 122a between the neuroregulator 104 and the lead assemblies 106, 106a, the lead assemblies 106, 106a may be implanted separately from the neuroregulator 104. Also, following implantation, the lead assemblies 106, 106a may be left in place while the originally implanted neuroregulator 104 is replaced by a different neuroregulator.

As shown in FIG. 2A, the neuroregulator connectors 122, 122a can be configured to receive connectors 126 of the lead assemblies 106, 106a. For example, the connectors 122, 122a of the neuroregulator 104 may be configured to receive pin connectors (not shown) of the lead assemblies 106, 106a. In another embodiment, the connectors 122, 122a may be configured to secure to the lead assemblies 106, 106a using first and second set-screws 123, 123a, respectively, or other such fasteners. In a preferred embodiment, the connectors 122, 122a are well-known IS-1 connectors. As used herein, the term “IS-1” refers to a connector standard used by the cardiac pacing industry, and is governed by the international standard ISO 5841-3.

In the example shown in FIG. 2B, first and second female connectors 122′, 122a′ are configured to receive the leads 106, 106a and molded into a portion of the overmold 110′ of the neuroregulator 104′. The lead connectors 126 are inserted into these molded connectors 122′, 122a′ and secured via first and second setscrews 123′, 123a′, seals (e.g., Bal Seals®)), and/or another fastener.

The circuit module 112 (see FIGS. 1, 3A, and 3B) is generally configured to generate therapy signals and to transmit the therapy signals to the lead assemblies 106, 106a. The circuit module 112 also may be configured to receive power and/or data transmissions from the external charger 101 via the internal coil 105. The internal coil 105 may be configured to send the power received from the external charger 101 to the circuit module 112 for use or to the internal power source (e.g., battery) 151 of the neuroregulator 104 to recharge the power source 151.

Block diagrams of example circuit modules 112, 112a are shown in FIGS. 3A and 3B, respectively. Either circuit module 112, 112a can be utilized with any neuroregulator, such as neuroregulators 104, 104′ described above. The circuit modules 112, 112a differ in that the circuit module 112a may be operated directly from a field programmable gate array 204, without the presence of a micro controller reducing its power consumption, and the circuit module 112 does not. Power operation for circuit module 112 may be provided by the external charger 101 or by the internal power source 151. Either circuit module 112, 112a may be used with either neuroregulator 104, 104′ shown in FIGS. 2A, 2B.

The circuit module 112 includes an RF input 157 including a rectifier 164. The rectifier 164 converts the RF power received from the internal coil 105 into DC electric current. Alternatively, alternating current can be used to provide a selectable but constant voltage or current. Circuitry for constant voltage or constant current devices is known to those of skill in the art.

For example, the RF input 157 may receive the RF power from the internal coil 105, rectify the RF power to DC power, and transmit the DC current to the internal power source 151 for storage. In one embodiment, the RF input 157 and the coil 105 may be tuned such that the natural frequency maximizes the power transferred from the external charger 101.

In an embodiment, the RF input 157 can first transmit the received power to a charge control module 153. The charge control module 153 receives power from the RF input 157 and delivers the power where needed through a first power regulator 156. For example, the RF input 157 may forward the power to the battery 151 for charging or to circuitry for use in creating therapy signals as will be described below. When no power is received from the coil 105, the charge control module 153 may draw power from the battery 151 and transmit the power through the second power regulator 160 for use. For example, a central processing unit (CPU) 154 of the neuroregulator 104 may manage the charge control module 153 to determine whether power obtained from the coil 105 should be used to recharge the power source 151 or whether the power should be used to produce therapy signals. The CPU 154 also may determine when the power stored in the power source 151 should be used to produce therapy signals.

The transmission of energy and data via RF/inductive coupling is known in the art. Further details describing recharging a battery via an RF/inductive coupling and controlling the proportion of energy obtained from the battery with energy obtained via inductive coupling can be found in the following references, all of which are hereby incorporated by reference herein: U.S. Pat. No. 3,727,616, issued Apr. 17, 1973, U.S. Pat. No. 4,612,934, issued Sep. 23, 1986, U.S. Pat. No. 4,793,353, issued Dec. 27, 1988, U.S. Pat. No. 5,279,292, issued Jan. 18, 1994, and U.S. Pat. No. 5,733,313, issued Mar. 31, 1998.

In general, the internal coil 105 may be configured to pass data transmissions between the external charger 101 and a telemetry module 155 of the neuroregulator 104. The telemetry module 155 generally converts the modulated signals received from the external charger 101 into data signals understandable by the CPU 154 of the neuroregulator 104. For example, the telemetry module 155 may demodulate an amplitude modulated carrier wave to obtain a data signal. In one embodiment, the signals received from the internal coil 105 are programming instructions from a physician (e.g., provided at the time of implant or on subsequent follow-up visits). The telemetry module 155 also may receive signals (e.g., patient data signals) from the CPU 154 and may send the data signals to the internal coil 105 for transmission to the external charger 101.

The CPU 154 may store operating parameters and data signals received at the neuroregulator 104 in an optional memory 152 of the neuroregulator 104. Typically, the memory 152 includes non-volatile memory. In other embodiments, the memory 152 also can store serial numbers and/or model numbers of the leads 106; serial number, model number, and/or firmware revision number of the external charger 101; and/or a serial number, model number, and/or firmware revision number of the neuroregulator 104.

The CPU 154 of the neuroregulator 104 also may receive input signals and produce output signals to control a signal generation module 159 of the neuroregulator 104. Signal generation timing may be communicated to the CPU 154 from the external charger 101 via the internal coil 105 and the telemetry module 155. In other embodiments, the signal generation timing may be provided to the CPU 154 from an oscillator module (not shown). The CPU 154 also may receive scheduling signals from a clock, such as 32 KHz real time clock (not shown).

The CPU 154 forwards the timing signals to the signal generation module 159 when therapy signals are to be produced. The CPU 154 also may forward information about the configuration of the electrode arrangement 108 to the signal generation module 159. For example, the CPU 154 can forward information obtained from the external charger 101 via the internal coil 105 and the telemetry module 155.

The signal generation module 159 provides control signals to an output module 161 to produce therapy signals. In an embodiment, the control signals are based at least in part on the timing signals received from the CPU 154. The control signals also can be based on the electrode configuration information received from the CPU 154.

The output module 161 produces the therapy signals based on the control signals received from the signal generation module 159. In an embodiment, the output module 161 produces the therapy signals by amplifying the control signals. The output module 161 then forwards the therapy signals to the lead arrangement 108.

In an embodiment, the signal generation module 159 receives power via a first power regulator 156. The power regulator 156 regulates the voltage of the power to a predetermined voltage appropriate for driving the signal generation module 159. For example, the power regulator 156 can regulate the voltage in a range of 0.01-20 volts.

In an embodiment, the output module 161 receives power via a second power regulator 160. The second power regulator 160 may regulate the voltage of the power in response to instructions from the CPU 154 to achieve specified constant voltage levels. The second power regulator 160 also may provide the voltage necessary to deliver constant current to the output module 161.

The output module 161 can measure the voltage of the therapy signals being outputted to the lead arrangement 108 and report the measured voltage to the CPU 154. A capacitive divider 162 may be provided to scale the voltage measurement to a level compatible with the CPU 154. In another embodiment, the output module 161 can measure the impedance of the lead arrangement 108 to determine whether the leads 106, 106a are in contact with tissue. This impedance measurement also may be reported to the CPU 154.

Another embodiment of a circuit is shown in FIG. 3B. The therapy algorithm is divided into a number of very small time segments and the corresponding voltage or current value of that therapy waveform segment is stored into a Field Programmable Gate Array 204. The therapy algorithm voltage or current values may be absolute values or changes relative to the previous voltage or current values. There is an option to retrieve alternate waveforms from an EEPROM 203. The clock oscillator 201 determines the time between successive therapy waveform segments and provides various clock signals for other circuits. The charge pump 205 provides the necessary voltage levels from the battery voltage for operating the circuits, the High Voltage (HV) generator 207 and a current source 208 provide the applicable voltage and current levels for the therapy waveform which may be programmable by the user. Various voltage monitors 202, regulators (not shown) and impedance detectors 206 measure and control the correct operation of the circuits. Some of the functionality is optional such as the memory 203 and telemetry blocks 155.

In addition, the power consumption needs of the neuroregulator 104, 104′ can change over time due to differences in activity. For example, the neuroregulator 104, 104′ will require less power to transmit data to the external charger 101 or to generate therapy signals than it will need to recharge the internal battery 151.

b. Electrodes

### Multipolar Electrodes

In embodiments, the disclosure provides a multipolar electrode assembly. Multipolar electrodes include, for example, a bipolar, a tripolar, a quadruple polar, and five polar electrode. One of the advantages of using multipolar electrodes is that rapid firing of action potentials at the beginning of HFAC may be reduced and sustained firing of action potentials during a prolonged application of HFAC may be minimized, resulting in a more effective block. A multipolar electrode has many advantages in terms of flexibility in procedures involving neuromodulation therapies.

### Tripolar Electrodes

In the case of using tripolar electrodes to deliver a high frequency alternating current (HFAC) neuronal conduction block, the outer two electrodes can have the same polarity, with the middle electrode having the opposite polarity of the outer two electrodes (tripolar configuration).

As an example of a tripolar electrode assembly, it may be desirable for only two of the electrodes to deliver HFAC and the third to act as a ground. In embodiments, the two electrodes delivering HFAC can either be adjacent (i.e., next to each other) or the outer two electrodes. In embodiments, if another electrode or electrode assembly is placed on another branch of a nerve, another nerve or anatomical feature, the device could be configured to send current from one of the electrode assemblies to the other in a monopolar configuration. A monopolar configuration could also be achieved by sending current from one, or both, of the electrode assemblies to the pulse generator at the same or different times.

In another embodiment using a tripolar electrode assembly, the electrodes could also be configured to stimulate. In embodiments, configurations of one polarity on the outer two electrodes and the opposite polarity on the middle electrode could be applied. Another configuration could include a bipolar configuration between two adjacent electrodes or a bipolar configuration between the outer two electrodes. The third electrode not delivering current could be grounded to the pulse generator.

An alternative embodiment of a tripolar electrode assembly could be configured to have the current flowing out of the electrodes to produce conduction block and directional stimulation. The inner and one of the outer electrodes could deliver HFAC while the other outer electrode is delivering stimulation. In one stimulation configuration, one pole would be the outer electrode and the other pole the pulse generator. If another electrode or electrode assembly is on another branch of a nerve, another nerve or anatomical feature, the opposite pole could be one or multiple electrodes in the other assembly. The assembly on another branch of a nerve, another nerve or anatomical feature could also be configured to be in a block and directional stimulation mode during the same time as the other assembly is doing the equivalent or is quiescent.

In the block and directional stimulation configuration, either afferent nerve fibers (axons that send information toward central nervous system) or efferent nerve fibers (axons that send information in a peripheral direction from the central nervous system) could be activated. If two tripolar electrode assemblies are on two different branches of a nerve, another nerve or anatomical feature, then they could independently (or concurrently) be in any of the above configurations which allows for a sink to a current source.

If two tripolar electrode assemblies are on two different branches of a nerve, another nerve or anatomical feature, then any of the above configurations could be applied with different temporal patterns. Examples include, but not limited to, blocking a first nerve and stimulating a second and then switching to stimulating the first nerve and blocking the second. Blocking one nerve and stimulating a second followed by blocking both nerves or vice versa. Blocking both nerves followed by stimulating both nerves or vice versa. Using directional block and stimulation on a nerve and then switching the direction of the stimulation and block. Using directional block and stimulation on two nerves and then switching the direction of the stimulation and block on one or both of the nerves.

### Five Polar Electrodes

A five polar electrode would be beneficial for various types of neuromodulation. In terms of HFAC conduction block a five polar electrode may help decrease onset responses or repetitive firing during long durations of HFAC. One configuration to do this would be the middle and outer two electrodes having the same polarity and the other two electrodes having the opposite polarity. In another configuration, the outer two electrodes would be grounded to the pulse generator, the middle electrode with one polarity and the adjacent electrodes to the middle having the opposite polarity. In another configuration, one of the outer electrodes and its adjacent electrode would be grounded to the pulse generator and the other three electrodes delivering HFAC in a tripolar configuration. Another grounding method would be to have an outer electrode and its adjacent electrode blocking in a bipolar configuration while the other three are grounded to the pulse generator. The five polar electrode assembly could also block in a monopolar configuration with one or multiple electrodes sending current to the pulse generator. A five polar electrode assembly can also be configured to stimulate. One configuration to do this would be the middle and outer two electrodes having the same polarity and the other two the opposite polarity. In another configuration the outer two electrodes would be grounded to the pulse generator, the middle electrode with one polarity and the adjacent electrodes to the middle having the opposite polarity. In another configuration one of the outer electrodes and its adjacent electrode would be grounded to the pulse generator and the other three electrodes stimulating in a tripolar configuration. Another grounding method would be to have an outer electrode and its adjacent electrode stimulating in a bipolar configuration while the other three are grounded to the pulse generator. The five polar electrode assembly could also stimulate in a monopolar configuration with one or multiple electrodes sending current to the pulse generator.

Directional block and stimulation could also be accomplished with a five polar electrode assembly. An outer and its adjacent electrode could be delivering stimulation in a bipolar configuration while the other three are delivering high HFAC conduction block in a tripolar configuration. Likewise, an outer and its adjacent electrode could be delivering HFAC conduction block in a bipolar configuration while the other three are delivering stimulation in atripolar configuration. An outer and its adjacent electrode could be delivering stimulation in a bipolar configuration while two out of the other three are delivering HFAC conduction block in a bipolar configuration while the other electrode (either the one next to the blocking electrodes or one next to the stimulation electrodes) is grounded to the pulse generator. In the block and directional stimulation configuration, either afferent nerve fibers (axons that send information toward central nervous system) or efferent nerve fibers (axons that send information in a peripheral direction from the central nervous system) could be activated.

If two five polar electrode assemblies are on two different branches of a nerve, another nerve or anatomical feature, a monopolar configuration could be achieved for stimulation or HFAC conduction block by sending current from one, or both, of the electrode assemblies to the pulse generator at the same or different times. A monopolar configuration could also be achieved by sending current from one, or more than one electrode of one assembly to one, or more than one electrode of the other assembly.

If two five polar electrode assemblies are on two different branches of a nerve, another nerve or anatomical feature, then they could independently (or concurrently) be in any of the above configurations which allows for a sink to a current source.

If two five polar electrode assemblies are on two different branches of a nerve, another nerve or anatomical feature, then any of the above configurations could be applied with different temporal patterns. Examples include, but not limited to, blocking a first nerve and stimulating a second and then switching to stimulating the first nerve and blocking the second. Blocking one nerve and stimulating a second followed by blocking both nerves or vice versa. Blocking both nerves followed by stimulating both nerves or vice versa. Using directional block and stimulation on a nerve and then switching the direction of the stimulation and block. Using directional block and stimulation on two nerves and then switching the direction of the stimulation and block on one or both of the nerves.

When low duty cycle electrical signal algorithms are applied, energy savings can be improved by also increasing impedance of the electrodes. Increasing impedance of the electrodes can be accomplished by varying electrode size and/or by coating electrodes with a coating having a resistivity that is at least 102×cm². For example, a 2500 Hz 90 microsecond algorithm is considered a low duty cycle (LDC) algorithm whereas a 5000 Hz 90 microsecond algorithm is considered a high duty cycle (HDC) algorithm. Note that the amount of current required to produce a 50% block is similar for the LDC and HDC algorithms with the high impedance range (at least 6000 Ohms). With a lower impedance range (3000-6000 Ohms) it takes more current to block 50% of the nerve with the LDC algorithm as with the HDC algorithm.

In other embodiments, the electrode size is such that the impedance between the tissue and the electrode is at least 2000 Ohms. In some embodiments, the electrode has a size of less than about 10 mm². Decreasing electrode size provides a higher impedance and lower energy requirements. Increasing impedance can improve the blocking effectiveness of a low duty cycle algorithm compared to a high duty cycle algorithm by shifting the current effect relationship curve (of blocking) for a low duty cycle algorithm closer to that of a high duty cycle algorithm (figure z). High impedance electrodes sizes, for example, can range from 0.1 to 9.99 mm². Electrodes having an impedance of at least 2000 Ohms can be employed in any of the multipolar configurations described herein. Smaller size electrodes may also decrease prolonged repetitive firing during HFAC.

In other embodiments, the impedance of the electrode is increased through the use of a coating that has a resistivity of at least 10² ohms per cm². Such coatings include Teflon, silicon, polyethylene, paralene as described in US20140214129, which is hereby incorporated by reference.

c. Biological Sensor

In some embodiments, the therapy system further comprises a biological sensor. The biological sensor may be an independent unit integrated into the therapy system, or be otherwise operatively coupled to the therapy system. In embodiments, the biological sensor is electrically connected to the therapy system. In embodiments, the biological sensor is in wireless communication with the therapy system. In embodiments, the biological sensor is operatively coupled to the neuroregulator of the therapy system. For example, a sensing electrode SE of the biological sensor can be added to monitor neural activity as a way to determine how to modulate the neural activity and/or the duty cycle. While sensing electrode can be an additional electrode to blocking electrode, it will be appreciated a single electrode could perform both functions. The sensing and blocking electrodes can be connected to a controller as shown in FIG. 1A and FIG. 1B. Such a controller is the same as controller 102 previously described with the additive function of receiving a signal from sensing electrode.

In some embodiments, the sensor can be a sensing electrode, a glucose sensor, or sensor that senses other biological molecules or hormones of interest. When the sensing electrode SE yields a signal representing a targeted maximum vagal activity or tone, the controller with the additive function of receiving a signal from sensing electrode functions to change and/or maintain the signals delivered to the electrode(s) placed on nerve branches/fibers. As described with reference to controller 102, controller with the additive function of receiving a signal from sensing electrode can be remotely programmed as to parameters of blocking/stimulating duration and no blocking/stimulation duration as well as targets for initiating, or maintaining, or ceasing, or terminating, or otherwise manipulating the blocking signal and/or upregulating signal.

As an exemplary example, a system comprises an implantable neuroregulator; at least one first electrode electrically connected to the implantable neuroregulator and adapted to be placed on a first nerve of a subject; at least one second electrode electrically connected to the implantable neuroregulator and adapted to be placed on a second nerve of the subject; and a glucose sensor, wherein the implantable neuroregulator comprises a microprocessor, the microprocessor configured to independently deliver a first electrical signal to the first nerve through the first electrode and deliver a second electrical signal to the second nerve through the second electrode, wherein the first electrical signal has parameters to downregulate nerve activity and the second electrical signal has parameters to stimulate nerve activity, and wherein the first electrical signal has a frequency of about 200 Hz to about 100 kHz, wherein the second electrical signal has a frequency of about 0.01 Hz to 199 Hz, and wherein the glucose sensor is configured to measure the blood glucose of the subject.

In practicing the therapy system, depending upon the glucose value of the subject indicated by the glucose sensor, the system can apply responsive changes to the first and/or the second electrical signal to control/maintain the blood glucose at a demanded level.

2. Electrical Signal Parameters

In some aspects, the present disclosure describes systems and methods of providing electrical signal therapy for downregulating and/or upregulating nerve activity in a subject. The systems and methods provide for layered patterns of electrical signal including microsecond inactive phases, millisecond inactive phases, and/or off times in order to vary how and when charge is applied to the nerve, and to save energy.

In some embodiments, the present system and method comprise providing electrical signal therapy for downregulating nerve activity in a subject. In other embodiments, the present system and method comprise providing low frequency stimulation signal therapy for upregulating nerve activity in a subject. In other embodiments, the present system and method comprise providing electrical signal therapy by combining downregulation of nerve activity of a nerve or a nerve and upregulation of nerve activity of a separate nerve or a separate nerve in the same subject. In certain embodiments, the present system and method comprises providing electrical signal therapy by concurrently downregulating nerve activity of a nerve or a nerve and upregulating nerve activity of a separate nerve or a separate nerve in the same subject.

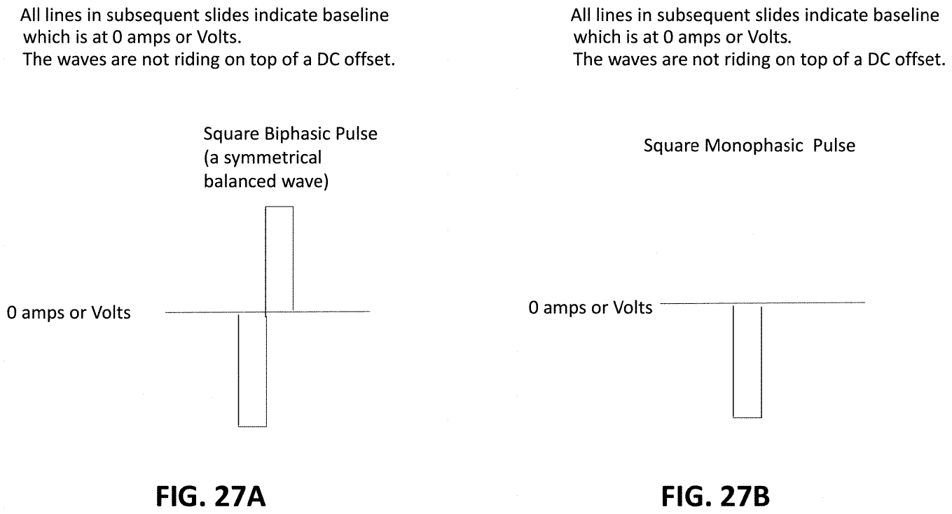

The waveform of the signals according to the present disclosure may be square, or trapezoidal, or sinusoidal, or exponential, or triangular, or stepwise, or combinations thereof. The pulse of the waveform can be monophasic, or biphasic, or polyphasic, or other shape. A monophasic shape is a single phase, unidirectional pulse from baseline to either positive or negative as illustrated in FIG. 27(A). A biphasic shape is a two phase, bidirectional wave with one negative phase and one positive phase as illustrated in FIG. 27(B).

A biphasic pulse could be symmetrical or charge balanced. A symmetrical biphasic pulse is any combination of two of monophasic charges/phases applied next to each other in which one is cathodic (negative) and the other anodic (positive), in either order, and the area under the curves are the same for the anodic aspect and cathodic aspect of the new pulse. A biphasic pulse could alternatively be asymmetrical or charge unbalanced. An asymmetrical biphasic pulse is any combination of two of the monophasic phases/charges applied next to each other between pulse in which one is cathodic (negative) and the other anodic (positive), in either order, and the area under the curves are different for the anodic aspect and cathodic aspect of the new pulse.

A polyphasic shape is bidirectional wave with three or more phases in bursts.

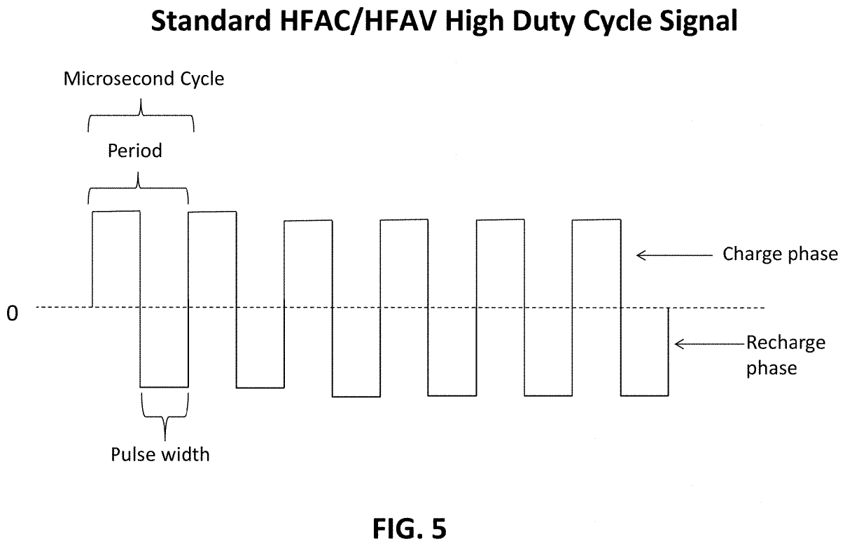

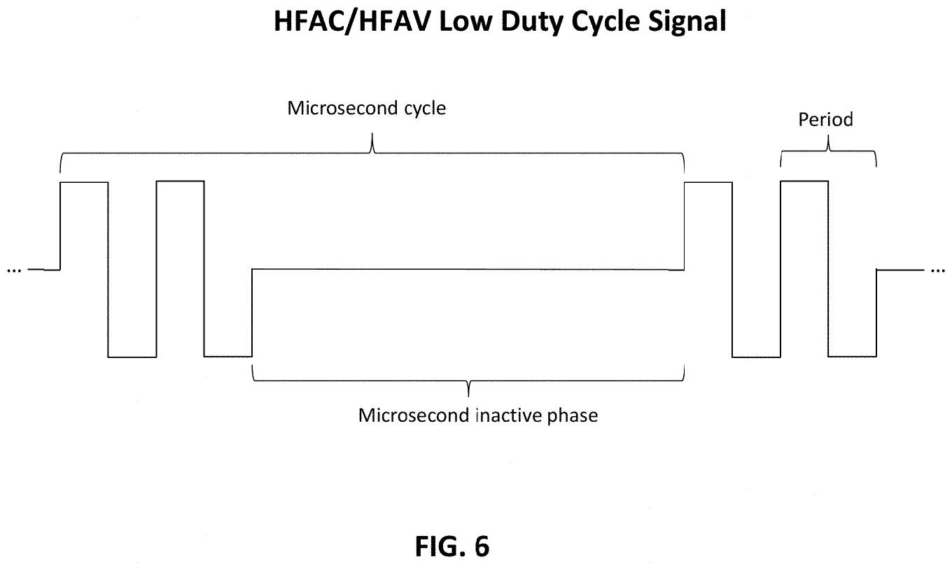

In some embodiments, the high frequency signal or high frequency low duty cycle signal have a biphasic waveform, comprising at least one pulse (charge recharge phase) having a positive phase at first and a subsequent negative phase in one pulse, as illustrated in FIG. 5 and FIG. 6.

In some embodiments, the low frequency stimulation signal has a monophasic waveform. In other embodiments, the low frequency stimulation signal has a biphasic waveform, comprising at least one pulse (cathodic and anodic phase) having an order of negative-positive in one pulse, as illustrated in FIG. 27(a). It was found that the low frequency stimulation signal having a biphasic waveform with an order of negative-positive in one pulse is more efficient in energy, producing prominent stimulation effects with relatively lower amplitude/voltage and therefore energy consumption, compared with the opposite order a.k.a., positive-negative.

It is importantly noted that the waveform and pulse patterns are not limited by the examples and embodiments provided herein. When practicing the present method and systems, especially the method using a combination of low frequency stimulation signal with high frequency blocking signal to regulate separate nerve/nerve branch/nerve fiber, other waveforms or patterns could also be used to improve the energy efficiency and effectiveness of electrical signals.

a. High Frequency Signal

In embodiments, a system and method of applying a high frequency signal comprises more than one microsecond cycle, each microsecond cycle comprising more than one period, each period comprising a charge and recharge phase and optionally, a pulse delay, each period having a frequency of at least about 200 Hz; and a microsecond inactive phase.

In other embodiments, a system and method of applying a high frequency signal comprises delivering more than one microsecond cycle to form a millisecond cycle, each millisecond cycle separated by a millisecond inactive phase. The length of time of the microsecond and/or millisecond inactive phases provides for the ability to vary how often electrical signal treatment is applied to the nerve during an on time and allows for energy savings as compared to electrical signal therapy not having inactive phases.

In embodiments, a system and method of applying a high frequency electrical signal having parameters that downregulate and/or upregulate nerve activity to a nerve in a subject comprises: applying the electrical signal to the nerve during an on time, wherein the electrical signal comprises more than one microsecond cycle comprising more than one period, each period comprising a charge recharge phase which may or may not have pulse delays, each period having a frequency of about at least 200 Hz; and a microsecond inactive phase. In embodiments, a microsecond cycle has a period comprising a charge and recharge phase, and optionally, includes one or more pulse delays. The period of a charge recharge phase is based on the frequency selected and the presence of pulse delays. For example, a charge recharge phase having a frequency of 5000 Hz without any pulse delay would have a period of at 200 microseconds based on 1 divided by the frequency. In other cases, the period of each charge and recharge phase for a frequency of 5000 Hz is 200 microseconds including a first pulse delay of 10 microseconds and a second pulse delay of 10 microseconds and a charge phase of 90 microseconds and recharge phase of 90 microseconds.

In some embodiments, a first pulse delay occurs after the charge phase and/or a second pulse delay occurs after the recharge phase. In embodiments, the first and second pulse delays are the same length. In embodiments, the length of the first and/or second pulse delay is selected to allow for a charge balanced alternating current signal to be delivered to the nerve. In embodiments, a pulse delay is about 30 microseconds or less. An exemplary embodiment is shown in FIG. 7. FIG. 7 shows three microsecond cycles, each microsecond cycle comprises a period comprising a charge phase followed by a pulse delay, a recharge phase and a pulse delay; and a microsecond inactive phase.

In embodiments, the electrical signal has a frequency in each period of a microsecond cycle of at least 200 Hz, at least 250 Hz, at least 300 Hz, at least 400 Hz, at least 500 Hz, at least 1000 Hz, at least 2000 Hz, at least 3000 Hz, at least 4000 Hz, or at least 5000 Hz, or at least 10,000 Hz, or at least 20,000 Hz, or at least 30,000 Hz, or at least 40,000 Hz, or at least 50,000 Hz, or at least 60,000 Hz, or at least 70,000 Hz, or at least 80,000 Hz, or at least 90,000 Hz, or at least 100,000 Hz, or at least 150 kHz, or at least 200 kHz, or at least 250 kHz or more. In other embodiments, the frequencies range from about 200 Hz to 250 kHz, 200 Hz to 200 kHz, 200 Hz to 150 kHz, 200 Hz to 100 kHz, 200 to 50 kHz, 200 Hz to 25 k Hz, 200 Hz to 10 kHz, or 200 Hz to 5000 Hz, or 200 Hz to 3000 Hz. or 200 Hz to 1500 Hz, or 200 to 1000 Hz. In other embodiments, the frequencies range from about 1000 Hz to 250 kHz, 1000 Hz to 200 kHz, 1000 Hz to 150 kHz, 1000 Hz to 100 kHz, 1000 to 50 kHz, or 1000 Hz to 25 kHz, or 1000 Hz to 10 kHz, or 1000 Hz to 5000 Hz, or 1000 Hz to 3000 Hz, or 1000 Hz to 1500 Hz, or 1000 Hz to 1000 Hz. In other embodiments, the frequencies range from about 200 Hz to 10 kHz, 200 Hz to 9000 Hz, 200 Hz to 8000 Hz, 200 Hz to 7000 Hz, 200 to 6000 Hz, 200 Hz to 5000 Hz, 200 Hz to 4000 Hz, 200 Hz to 3000 Hz, or 200 Hz to 2000 Hz, or 200 Hz to 1000 Hz. In other embodiments, the frequencies range from about 1000 Hz to 10 kHz, 1000 Hz to 9000 Hz, 1000 Hz to 8000 Hz, 1000 Hz to 7000 Hz, 1000 to 6000 Hz, 1000 Hz to 5000 Hz, 1000 Hz to 4000 Hz, 1000 Hz to 3000 Hz, or 1000 Hz to 2000 Hz. In embodiments, electrical signals at such frequencies can downregulate nerve activity.

In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can stimulate nerve activity.

In embodiments, the amplitude of the signal is at least 1 mAmp. In other embodiments, the amplitude ranges from about 0.1 to 20 mAmps, 0.1 to 15 mAmps, 0.1 to 10 mAmps, 0.1 to 8 mAmps, or 0.1 to 5 mAmps.

In embodiments, the amplitude is at least 1 volt. In other embodiments, the amplitude ranges from about 1 to 20 volts, 1 to 15 volts, 1 to 10 volts, 1 to 8 volts, or 1 to 5 volts.

In embodiments, the on time is at least about 30 seconds. In other embodiments, the on time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow at least partial recovery of the nerve. In embodiments, the off time may be minimized due to the presence of microsecond inactive phases and/or millisecond inactive phases. In embodiments, off times are at least about 30 seconds. In other embodiments, the off time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include off times of varying amounts. For example, a therapy cycle can include 1 minute of on time, 1 minute of off time, 2 minutes of on time, followed by 5 minutes of off time.

In embodiments, the microsecond cycle comprises more than one period, each period comprising a charge recharge phase and may or may not contain pulse delays; and a microsecond inactive phase. In some embodiments, the inactive phase is longer than the period. In embodiments, the length of the inactive phase can vary between each period.

In embodiments, the period is about 1000 microseconds or less, about 500 microseconds or less, or about 200 microseconds or less.

In embodiments, the microsecond inactive phase is in a ratio to the charge recharge phase of about 10 to 1, 8 to 1, 6 to 1, 4 to 1, or 2 to 1. In embodiments, the microsecond inactive phase is at least about 80 microseconds. In embodiments, the microsecond inactive phase is at least 80 microseconds up to 10,000 microseconds, 200 microseconds up to 10,000 microseconds, or 400 microseconds up to 10,000 microseconds. In embodiments, the microsecond inactive phase is about 10 microseconds to 10,000 microseconds. In embodiments, a microsecond inactive phase is 10,000 microseconds or less, 1000 microseconds or less, or 500 microseconds or less. In embodiments, the microsecond inactive phase is at least 20 microseconds up to 10,000 microseconds, 20 microseconds up to 5000 microseconds, 20 microseconds up to 1000 microseconds, 20 microseconds up to 500 microseconds, or 20 microseconds up to 100 microseconds.

In embodiments, the frequency is at least 1000 Hz, 2000 Hz, 3000 Hz, 4000 Hz, 5000 Hz, 6000 Hz, 7000 Hz, 8000 Hz, 9000 Hz, or 10,000 Hz or more.

In embodiments, multiple periods can be administered in a single microsecond cycle. In other embodiments, the application of the electrical signal includes multiple microsecond cycles.

An exemplary embodiment is shown in FIG. 6. In FIG. 6, 2 microsecond cycles are shown. The first microsecond cycle comprises 2 periods, and a microsecond inactive phase. Each charge recharge phase in the microsecond cycle has a period equal to 1 divided by the frequency without any pulse delays. Energy savings are realized by including microsecond inactive phases between the periods as can be seen by comparison with FIG. 5. In FIG. 5, the standard HFAC therapy involves application of charge recharge phases during an on time without any microsecond inactive phases. In addition, the length of the microsecond inactive phases and/or the number of periods in a microsecond cycle can be varied to provide application of a total amount of charge during an on time while varying the impact on the nerve.

In other embodiments, a system and method of applying an electrical signal having parameters that downregulate and/or upregulate nerve activity to a nerve in a subject comprises: applying the electrical signal to the nerve during an on time, wherein the electrical signal comprises more than one microsecond cycle to form a millisecond active phase, and applying more than one millisecond active phase during the on time, wherein each millisecond active phase is separated by a millisecond inactive phase during the on time.

In embodiments, the electrical signal has a frequency in each period of a microsecond cycle of at least 200 Hz, at least 250 Hz, at least 300 Hz, at least 400 Hz, at least 500 Hz, at least 1000 Hz, at least 2000 Hz, at least 3000 Hz, at least 4000 Hz, or at least 5000 Hz, or at least 10,000 Hz, or at least 20,000 Hz, or at least 30,000 Hz, or at least 40,000 Hz, or at least 50,000 Hz, or at least 60,000 Hz, or at least 70,000 Hz, or at least 80,000 Hz, or at least 90,000 Hz, or at least 100,000 Hz, or at least 150 kHz, or at least 200 kHz, or at least 250 kHz or more. In other embodiments, the frequencies range from about 200 Hz to 250 kHz, 200 Hz to 200 kHz, 200 Hz to 150 kHz, 200 Hz to 100 kHz, 200 to 50 kHz, 200 Hz to 25 k Hz, 200 Hz to 10 kHz, or 200 Hz to 5000 Hz, or 200 Hz to 3000 Hz. or 200 Hz to 1500 Hz, or 200 to 1000 Hz. In other embodiments, the frequencies range from about 1000 Hz to 250 kHz, 1000 Hz to 200 kHz, 1000 Hz to 150 kHz, 1000 Hz to 100 kHz, 1000 to 50 kHz, or 1000 Hz to 25 kHz, or 1000 Hz to 10 kHz, or 1000 Hz to 5000 Hz, or 1000 Hz to 3000 Hz, or 1000 Hz to 1500 Hz, or 1000 Hz to 1000 Hz. In other embodiments, the frequencies range from about 200 Hz to 10 kHz, 200 Hz to 9000 Hz, 200 Hz to 8000 Hz, 200 Hz to 7000 Hz, 200 to 6000 Hz, 200 Hz to 5000 Hz, 200 Hz to 4000 Hz, 200 Hz to 3000 Hz, or 200 Hz to 2000 Hz, or 200 Hz to 1000 Hz. In other embodiments, the frequencies range from about 1000 Hz to 10 kHz, 1000 Hz to 9000 Hz, 1000 Hz to 8000 Hz, 1000 Hz to 7000 Hz, 1000 to 6000 Hz, 1000 Hz to 5000 Hz, 1000 Hz to 4000 Hz, 1000 Hz to 3000 Hz, or 1000 Hz to 2000 Hz. In embodiments, electrical signals at such frequencies can downregulate nerve activity.

In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can stimulate nerve activity.

In embodiments, the amplitude of the signal is at least 1 mAmp. In other embodiments, the amplitude ranges from about 1 to 20 mAmps, 1 to 15 mAmps, 1 to 10 mAmps, 1 to 8 mAmps, or 1 to 5 mAmps.

In embodiments, the amplitude is at least 1 volt. In other embodiments, the amplitude ranges from about 1 to 20 volts, 1 to 15 volts, 1 to 10 volts, 1 to 8 volts, or 1 to 5 volts.

In embodiments, the on time is at least about 30 seconds. In other embodiments, the on time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow at least partial recovery of the nerve. In embodiments, the off time may be minimized due to the presence of microsecond inactive phases and/or millisecond inactive phases. In embodiments, off times are at least about 30 seconds. In other embodiments, the off time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include off times of varying amounts. For example, a therapy cycle can include 1 minutes of on time, 1 minute of off time, 2 minutes of on time, followed by 5 minutes of off time.

In embodiments, a microsecond cycle has a period comprising a charge and recharge phase, and optionally, includes one or more pulse delays. The time of a period includes the time of a charge recharge phases and the presence or absence of pulse delays. For example, a period with a single charge recharge phase without any pulse delays and having a frequency of 5000 Hz has a period of 200 microseconds based on 1 divided by the frequency. In other cases, the period of each charge and recharge phase for a frequency of 5000 Hz is 200 microseconds including a 90 microsecond charge phase followed by a first 10 microsecond pulse delay, followed by a 90 microsecond discharge phase and a second pulse delay of 10 microseconds.

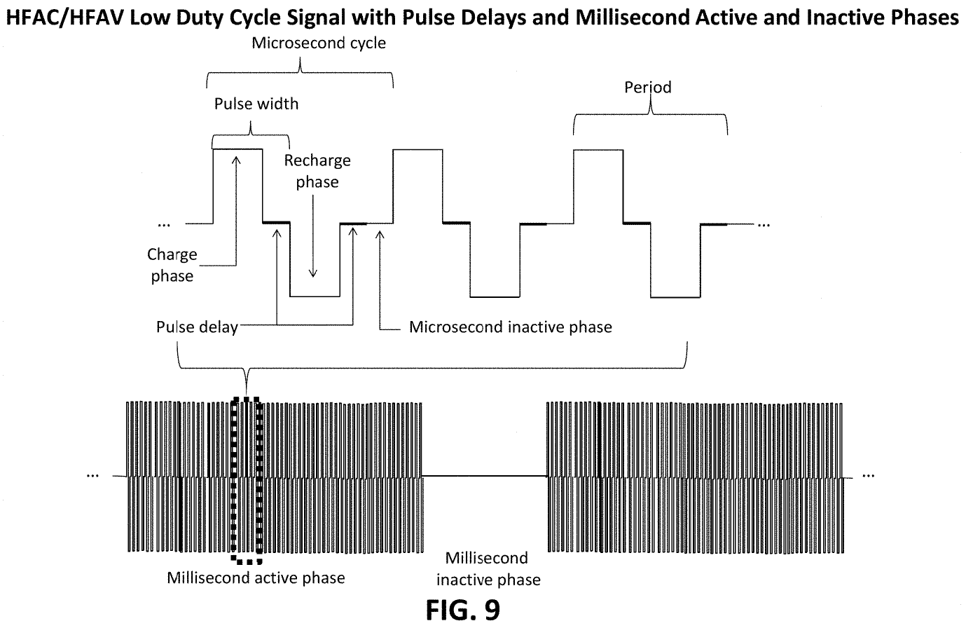

In some embodiments, a first pulse delay occurs after the charge phase and/or a second pulse delay occurs after the recharge phase. In embodiments, the first and second pulse delays are the same length. In embodiments, the length of the first and/or second pulse delay is selected to allow for a charge balanced alternating current signal to be delivered to the nerve and without sending unwanted signals. In embodiments, a pulse delay is about 30 microseconds or less. An exemplary embodiment is shown in FIG. 9. FIG. 9 shows three microsecond cycles, each microsecond cycle comprises a charge phase followed by a pulse delay, a recharge phase and a pulse delay; and a microsecond inactive phase. Multiple microsecond cycles form a millisecond active phase.

In embodiments, a millisecond active phase is separated from another millisecond active phase by a millisecond inactive phase. In embodiments, the millisecond inactive phase is longer than the millisecond active phase. In embodiments, the millisecond inactive phase can vary in time between each millisecond active phase.

In embodiments, the millisecond active phase is at least 0.16 millisecond. In embodiments, the millisecond active phase is 0.16 millisecond to 1,100 milliseconds, 0.16 millisecond to 900 milliseconds, 0.16 millisecond to 800 milliseconds, 0.16 millisecond to 700 milliseconds, 0.16 millisecond to 600 milliseconds. 0.16 millisecond to 500 milliseconds, 0.16 to 400 milliseconds, 0.16 to 300 milliseconds, 0.16 to 200 milliseconds, 0.16 to 100 milliseconds, 0.16 to 50 milliseconds, 0.16 to 40 milliseconds, 0.16 to 30 milliseconds, 0.16 to 20 milliseconds, 0.16 to 10 milliseconds, or 0.16 to 5 milliseconds.

In embodiments, the millisecond active phase is at least 1 millisecond. In other embodiments, the millisecond active phase is 1 to 1,100 milliseconds, 1 millisecond to 900 milliseconds, 1 millisecond to 800 milliseconds, 1 millisecond to 700 milliseconds, 1 millisecond to 600 milliseconds, 1 millisecond to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, 1 to 10 milliseconds, or 1 to 5 milliseconds.

In embodiments, the millisecond active phase comprises at least 2 to 100 microsecond cycles, at least 2 to 90, at least 2 to 80, at least 2 to 70, at least 2 to 60, at least 2 to 50, at least 2 to 40, at least 2 to 30, at least 2 to 20, at least 2 to 10, at least 2 to 5, or at least 2 to 4 microsecond cycles.

In embodiments, the millisecond inactive phase is in a ratio to the millisecond active phase of about 10 to 1, 8 to 1, 6 to 1, 4 to 1, 2 to 1 or 1 to 2. In embodiments, the millisecond inactive phase is at least 0.08 milliseconds. In embodiments, the millisecond inactive phase is 0.08 millisecond to 11,000 milliseconds, 0.08 millisecond to 9000 milliseconds, 0.08 millisecond to 8000 milliseconds, 0.08 millisecond to 7000 milliseconds, 0.08 millisecond to 6000 milliseconds, 0.08 millisecond to 5000 milliseconds, 0.08 to 4000 milliseconds, 0.08 to 3000 milliseconds, 0.08 to 2000 milliseconds, 0.08 to 1000 milliseconds, 0.08 to 500 milliseconds, 0.08 to 400 milliseconds, 0.08 to 300 milliseconds, 0.08 to 200 milliseconds, 0.08 to 100 milliseconds, 0.08 to 50 milliseconds, 0.08 to 40 milliseconds, 0.08 to 30 milliseconds, 0.08 to 20 milliseconds, or 0.08 to 10 milliseconds. In embodiments, the millisecond inactive phase is 1 millisecond to 11,000 milliseconds, 1 millisecond to 9000 milliseconds, 1 millisecond to 8000 milliseconds, 1 millisecond to 7000 milliseconds, 1 millisecond to 6000 milliseconds, 1 millisecond to 5000 milliseconds, 1 to 4000 milliseconds, 1 to 3000 milliseconds, 1 to 2000 milliseconds, 1 to 1000 milliseconds, 1 to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, or 1 to 10 milliseconds.

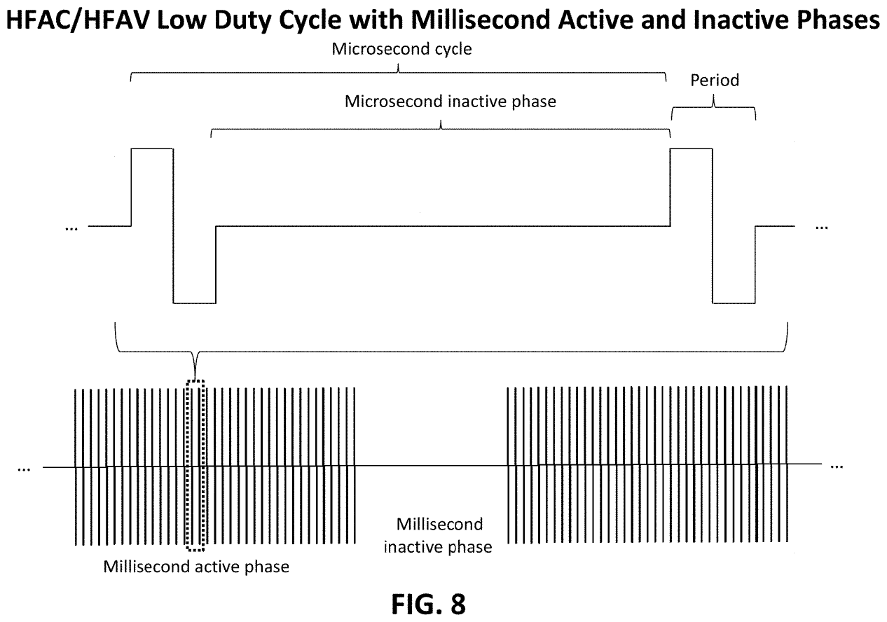

An exemplary embodiment is shown in FIG. 8. As shown in FIG. 8, a microsecond cycle comprises at least one period; and a microsecond inactive phase. The millisecond cycle comprises a millisecond active phase that includes more than one microsecond cycles and a millisecond inactive phase. Energy savings are realized by including microsecond inactive phases between the charge recharge phases as well as between millisecond inactive phases between millisecond active phases. In addition, the length of the microsecond inactive phases, millisecond inactive phases and/or the number of periods can be varied to provide application of a total amount of charge during an on time while varying the impact on the nerve. In embodiments, the frequency of the electrical signal treatment is selected to downregulate activity on the nerve and is at least 200 Hz, 300 Hz, 400 Hz, 500 Hz, 600 Hz, 700 Hz, 800 Hz, 900 Hz, 1000 Hz or more.

In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can stimulate nerve activity.

In yet other embodiments, a system and method of applying an electrical signal having parameters to downregulate and/or upregulate nerve activity to a nerve in a subject comprises: applying the electrical signal to the nerve during an on time, wherein the electrical signal comprises a first pattern and a second pattern which differ from one another. In embodiments, the first pattern comprises at least one microsecond cycle. In other embodiments, the first pattern comprises more than one millisecond active phase, wherein each millisecond active phase comprises more than one microsecond cycle, and each millisecond active phase is separated by a millisecond inactive phase. In embodiments, the second pattern comprises at least one microsecond cycle. In embodiments, the second pattern comprises more than one millisecond active phase, wherein each millisecond active phase comprises more than one microsecond cycle, and each millisecond active phase is separated by a millisecond inactive phase.

In yet other embodiments, a system and method of applying an electrical signal having parameters to downregulate and/or upregulate nerve activity to a nerve in a subject comprises: applying the electrical signal to the nerve during an on time, wherein the electrical signal comprises a first pattern comprising at least one microsecond cycle; and a second pattern comprising more than one millisecond active phase, wherein each millisecond active phase comprises more than one microsecond cycle, and each millisecond active phase is separated by a millisecond inactive phase, wherein the first and second patterns have a different amplitude and/or different on times. In embodiments, the microsecond cycle comprises at least one period comprising a charge recharge phase and optionally, a pulse delay, wherein each period has a frequency of about 0.01 Hz to about 5,000 Hz; and a microsecond inactive phase.

In embodiments, the electrical signal has a frequency of a period which comprises a charge recharge phase and may have pulse delays, wherein the frequency is at least 200 Hz, at least 250 Hz, at least 300 Hz, at least 400 Hz, at least 500 Hz, at least 1000 Hz, at least 2000 Hz, at least 3000 Hz, at least 4000 Hz, or at least 5000 Hz. In other embodiments, the frequencies range from about 200 Hz to 250 kHz, 200 Hz to 200 kHz, 200 Hz to 150 kHz, 200 Hz to 100 kHz, 200 to 50 kHz, 200 Hz to 25 k Hz, 200 Hz to 10 kHz, or 200 Hz to 5000 Hz, or 200 Hz to 3000 Hz, or 200 Hz to 1500 Hz, or 200 to 1000 Hz. In other embodiments, the frequencies range from about 1000 Hz to 250 kHz, 1000 Hz to 200 kHz, 1000 Hz to 150 kHz, 1000 Hz to 100 kHz, 1000 to 50 kHz, or 1000 Hz to 25 kHz, or 1000 Hz to 10 kHz, or 1000 Hz to 5000 Hz, or 1000 Hz to 3000 Hz, or 1000 Hz to 1500 Hz, or 1000 Hz to 1000 Hz. In other embodiments, the frequencies range from about 200 Hz to 10 kHz, 200 Hz to 9000 Hz, 200 Hz to 8000 Hz, 200 Hz to 7000 Hz, 200 to 6000 Hz, 200 Hz to 5000 Hz, 200 Hz to 4000 Hz, 200 Hz to 3000 Hz, or 200 Hz to 2000 Hz, or 200 Hz to 1000 Hz. In other embodiments, the frequencies range from about 1000 Hz to 10 kHz, 1000 Hz to 9000 Hz, 1000 Hz to 8000 Hz, 1000 Hz to 7000 Hz, 1000 to 6000 Hz, 1000 Hz to 5000 Hz, 1000 Hz to 4000 Hz, 1000 Hz to 3000 Hz, or 1000 Hz to 2000 Hz. In embodiments, electrical signals at such frequencies can downregulate nerve activity. In embodiments, electrical signals at such frequencies can downregulate or block nerve activity.

In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can stimulate or upregulate nerve activity.

In embodiments, the amplitude of the signal is at least 1 mAmp. In other embodiments, amplitudes ranges from about 1 to 20 mAmps, 1 to 15 mAmps, 1 to 10 mAmps, 1 to 8 mAmps, or 1 to 5 mAmps.

In embodiments, the amplitude is at least 1 volt. In other embodiments, the amplitude ranges from about 1 to 20 volts, 1 to 15 volts, 1 to 10 volts, 1 to 8 volts, or 1 to 5 volts.

In embodiments, the on time is at least about 30 seconds. In other embodiments, the on time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow at least partial recovery of the nerve. In embodiments, the off time may be minimized due to the presence of microsecond inactive phases and/or millisecond inactive phases. In embodiments, off times are at least about 30 seconds. In other embodiments, the off time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include off times of varying amounts. For example, a therapy cycle can include 1 minutes of on time, 1 minute of off time, 2 minutes of on time, followed by 5 minutes of off time.

In embodiments, the first pattern has an amplitude greater than the second pattern. In embodiments, the ratio of the amplitude of the first pattern to the amplitude of the second pattern is at least 10 to 1, 8 to 1, 6 to 1, 4 to 1, 2 to 1 or 4 to 3. In embodiments, the amplitude of the first embodiment is maintained or held constant during the time the first pattern is applied. In other embodiments, the amplitude of the second embodiment, while different than the first pattern, is maintained or held constant during the time period the second pattern of electrical signal is applied.

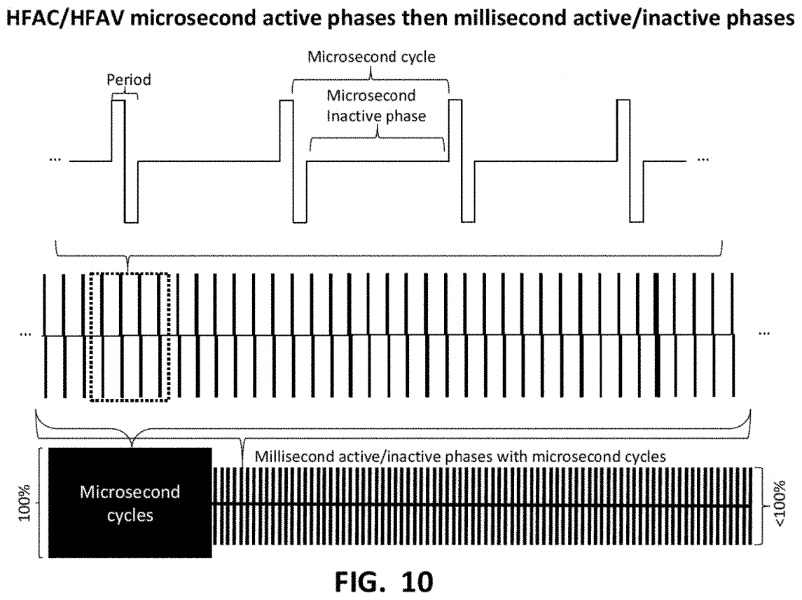

An exemplary embodiment is shown in FIG. 10. FIG. 10 shows a first pattern of electrical signals that comprise more than one microsecond cycle, each microsecond cycle having at least one period and a microsecond inactive phase. The period of each microsecond cycle is 5000 microseconds or less. The amplitude of the microsecond cycles is at least about 1 to 20 mAmps, 1 to 15 mAmps, 1 to 10 mAmps, 1 to 8 mAmps, or 1 to 5 mAmps.

FIG. 10 shows a second pattern of electrical signals that comprise one or more millisecond active phases, each millisecond active phase comprising one or more microsecond cycles. Each millisecond active phase has an amplitude that is different than the first pattern. The amplitude of the microsecond cycles in the millisecond active phase is at least about 1 to 20 mAmps, 1 to 15 mAmps, 1 to 10 mAmps, 1 to 8 mAmps, or 1 to 5 mAmps.

In any of the systems and methods described herein, application of an electrical signal can be initiated or terminated using a ramp up and/or ramp down of amplitude and/or pulse width and/or frequency. In embodiments, such ramp up and ramp down times are useful to minimize sensations or discomfort from application of an electrical signal to a nerve. In embodiments, a ramp up includes multiple pulses, each pulse has an increasing increment of amplitude and/or an increasing increment of pulse width and/or a decreasing increment of frequency. In embodiments, a ramp down includes multiple pulses, each pulse has a decreasing increment of amplitude and/or a decreasing increment of pulse width and/or a decreasing increment of frequency.

The amplitudes can range from about 0.1 to 20 mA, 0.1 to 20 mAmps, 0.1 to 15 mAmps, 0.1 to 10 mAmps, 0.1 to 8 mAmps, or 0.1 to 5 mAmps. In a ramp up, the initial amplitude can be higher than 0.1, for example starting at 3 mAmps. In a ramp down, the initial amplitude can be lower than 20, for example starting at 10 mAmps. In ramp up and/or ramp down the amplitude is changing during the ramp up or down time, whereas in the other methods described herein once a specific amplitude is reached it is maintained for the duration of the first pattern, followed by change to the second amplitude which is then maintained for the duration of the second pattern. In some embodiments, the time period for a ramp up and/or ramp down is about 120 microseconds to 11,000 milliseconds. In some embodiments, a ramping up of the amplitude occupies all of the time of the on time of a first and/or second pattern.

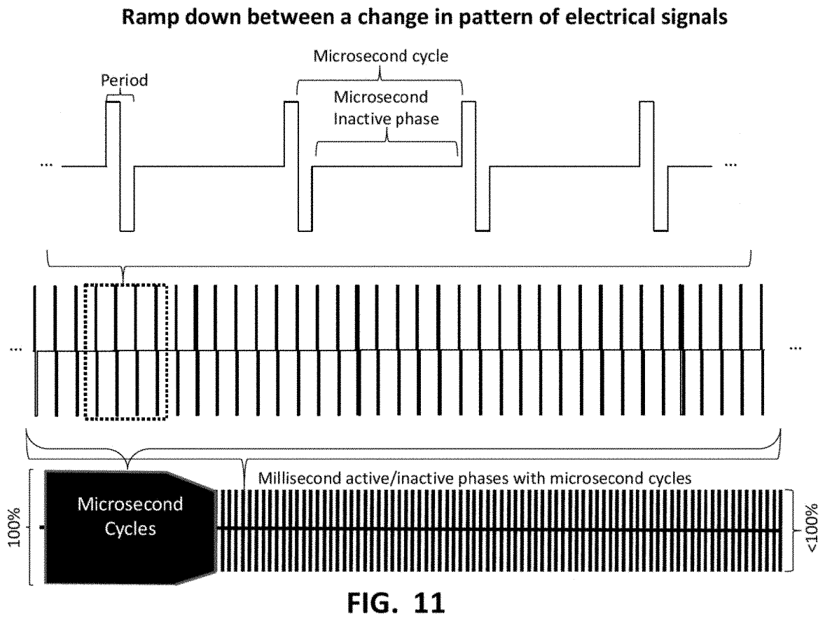

An exemplary embodiment is shown in FIG. 11. FIG. 11 shows a first pattern of electrical signals that comprise more than one microsecond cycle, each microsecond cycle having at least one period and a microsecond inactive phase. The period of each microsecond cycle is at least 5000 microseconds. The amplitude of the microsecond cycles is at least about 1 to 20 mAmps, 1 to 15 mAmps, 1 to 10 mAmps, 1 to 8 mAmps, or 1 to 5 mAmps. FIG. 11 shows a ramp down in amplitude from the first pattern to the amplitude of the second pattern. The change is amplitude is applied in increments.

FIG. 11 shows a second pattern of electrical signals that are applied after a ramp down and that comprise one or more millisecond active phases, each millisecond active phase comprising one or more microsecond cycles. Each millisecond active phase has an amplitude that is different than the first pattern. The amplitude of the microsecond cycles in the millisecond active phase is at least about 1 to 20 mAmps, 1 to 15 mAmps, 1 to 10 mAmps, 1 to 8 mAmps, or 1 to 5 mAmps.

b. Low Frequency Stimulation Signal

In embodiments, a system and method of applying a low frequency stimulation signal comprises at least one stimulation cycle, wherein each of the at least one stimulation cycle comprises at least one stimulation period, each of the at least one stimulation period comprising at least one pulse and optionally a stimulation inactive phase, wherein the pulse comprises a cathodic and/or anodic phase and optionally a pulse delay, the pulse having a pulse width. In embodiments, the low frequency stimulation signal is in a range from about 0.01 Hz to about 100 Hz, preferably from about 0.01 Hz to about 30 Hz.

In other embodiments, a system and method of applying a low frequency signal comprises delivering more than one stimulation active cycle to form a stimulation active phase, each stimulation active phase separated by an idle phase. The length of time of the stimulation inactive phases and/or idle provides for the ability to vary how often electrical signal treatment is applied to the nerve during an on time and allows for energy savings as compared to low frequency electrical signal therapy not having inactive phases.

In embodiments, a system and method of applying a low frequency stimulation signal having parameters that upregulate/stimulate nerve activity to a nerve in a subject comprises: at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width, and wherein the low frequency stimulation signal is in a range from about 0.01 Hz to about 100 Hz, preferably from about 0.01 Hz to about 30 Hz.

In embodiments, a stimulation active cycle has a stimulation period comprising a pulse, and optionally, includes one or more pulse delays. The stimulation period of a pulse is based on the frequency selected and the presence of pulse delays. For example, a pulse having a frequency of 100 Hz without any pulse delay would have a period of at 10 milliseconds based on 1 divided by the frequency.

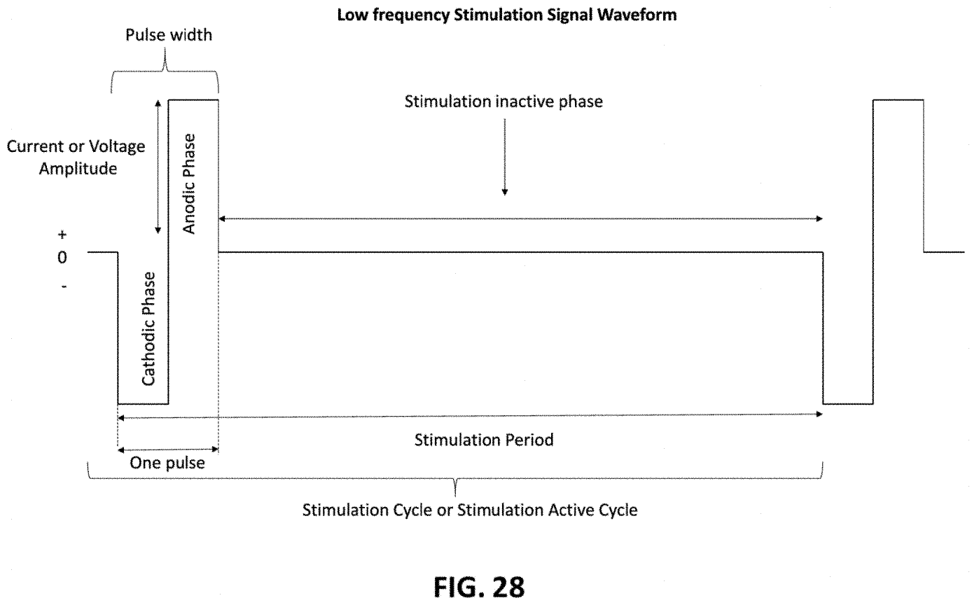

An exemplary embodiment is shown in FIG. 28. A stimulation cycle comprises at least one stimulation period; each stimulation period comprising at least one pulse having a negative (cathodic) charge phase followed by a positive (anodic) charge phase, and an optionally a stimulation inactive phase.

In some embodiments, a first pulse delay occurs after the negative (or cathodic) phase and/or a second pulse delay occurs after the positive (or anodic) phase. In embodiments, the first and second pulse delays are the same length. In embodiments, the length of the first and/or second pulse delay is selected to allow for a charge balanced alternating current signal to be delivered to the nerve.

In some embodiments, the pulse of the low frequency simulation signal is a monophasic phase.

In some embodiments, the pulse width of the low frequency simulation signal is from about 50 microseconds to about 10,000 microseconds, or from about 50 microseconds to about 8,000 microseconds, or from about 50 microseconds to about 6,000 microseconds, or from about 50 microseconds to about 4,000 microseconds, or from about 50 microseconds to about 2,000 microseconds, or from about 50 microseconds to about 1,000 microseconds, or from about 50 microseconds to about 500 microseconds, or from about 50 microseconds to about 100 microseconds, or from about 100 microseconds to about 10,000 microseconds, or from 500 microseconds to about 10,000 microseconds, or from about 1,000 microseconds to about 10,000 microseconds, or from about 2,000 microseconds to about 10,000 microseconds, or from about 4,000 microseconds to about 10,000 microseconds, or from about 6,000 microseconds to about 10,000 microseconds, or from about 8,000 microseconds to about 10,000 microseconds.

In embodiments, the low frequency stimulation signal has a frequency in each period of a stimulation active cycle of at most 199 Hz, at most about 150 Hz, at most about 100 Hz, at most about 50 Hz, at most about 40 Hz, at most about 30 Hz, at most about 20 Hz, at most about 10 Hz, at most about 1 Hz, at most about 0.5 Hz, at most about 0.1 Hz, at most about 0.05 Hz, or at most about 0.01 Hz or less. In other embodiments, the frequencies range from about 0.01 Hz to 199 Hz, or from about 0.01 Hz to about 100 Hz, or from about 0.01 Hz to about 50 Hz, or from about 0.01 Hz to about 30 Hz, or from about 0.01 Hz to about 10 Hz. In embodiments, low frequency stimulation signals at such frequencies can upregulate nerve activity.

In embodiments, the amplitude of the signal is at least 0.01 mAmp. In other embodiments, the amplitude ranges from about 0.01 to 20 mAmps, 0.01 to 15 mAmps, 0.01 to 10 mAmps, 0.01 to 8 mAmps, or 0.01 to 5 mAmps.

In embodiments, the amplitude is at least 0.014 volt. In other embodiments, the amplitude ranges from about 0.01 to 20 volts, 0.01 to 15 volts, 0.01 to 10 volts, 0.01 to 8 volts, or 0.01 to 5 volts.

In embodiments, the on time is at least about 30 seconds. In other embodiments, the on time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow pulsatile stimulation of the nerve with improved efficiency and reduced energy consumption. In embodiments, the off time may be minimized due to the presence of stimulation inactive phases and/or idle phases. In embodiments, off times are at least about 30 seconds. In other embodiments, the off time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include off times of varying amounts. For example, a therapy cycle can include 1 minute of on time, 1 minute of off time, 2 minutes of on time, followed by 5 minutes of off time.

In embodiments, the stimulation cycle comprises more than one stimulation period, each stimulation period comprising a pulse and may or may not contain pulse delays; and a stimulation inactive phase. In embodiments, the length of the stimulation inactive phase can vary between each stimulation period.

In embodiments, the stimulation period is about 0.01 seconds to about 100 seconds. In embodiments, a stimulation inactive phase is about 100 seconds or less, about 50 seconds or less, or about 10 second or less, or about 5 seconds or less, or about 1 second or less, or about 0.1 seconds or less, or about 0.01 seconds or less. In embodiments, the stimulation period is at least about 0.01 seconds up to 100 seconds, 0.01 seconds up to 50 seconds, 0.01 seconds up to 10 seconds, 0.01 seconds up to 5 seconds, or 0.01 seconds up to 1 second, or 0.01 seconds up to 0.5 seconds, or 0.01 seconds up to 0.2 seconds, or 0.01 seconds up to 0.1 seconds.

In embodiments, the stimulation inactive phase is in a ratio to the pulse width of about 1000 to 1, 500 to 1, 100 to 1, 50 to 1, 10 to 1, 5 to 1, or 4 to 1. In embodiments, the stimulation inactive phase is at least about 0.01 seconds, or about 0.02, or about 0.03 seconds. In embodiments, the stimulation inactive phase is at least 0.01 seconds up to 100 seconds, 0.1 seconds up to 100 seconds, or 1 second up to 100 seconds, or 5 seconds up to 100 seconds, or 10 seconds up to 100 seconds.

In embodiments, the stimulation inactive phase is about 0.01 seconds to about 100 seconds. In embodiments, a stimulation inactive phase is about 100 seconds or less, about 50 seconds or less, or about 10 second or less, or about 5 seconds or less, or about 1 second or less, or about 0.1 seconds or less, or about 0.01 seconds or less. In embodiments, the stimulation inactive phase is at least 0.01 seconds up to 100 seconds, 0.01 seconds up to 50 seconds, 0.01 seconds up to 10 seconds, 0.01 seconds up to 5 seconds, or 0.01 seconds up to 1 second, or 0.01 seconds up to 0.5 seconds, or 0.01 seconds up to 0.2 seconds, or 0.01 seconds up to 0.1 seconds.

In embodiments, the frequency is at most 199 Hz, at most 150 Hz, at most 100 Hz, at most 50 Hz, at most 40 Hz, at most 30 Hz, at most 20 Hz, at most 10 Hz, at most 1 Hz, at most 0.5 Hz, at most 0.1 Hz, at most 0.05 Hz, or at most 0.01 Hz or less.

In embodiments, multiple stimulation periods can be administered in a single stimulation cycle. In other embodiments, the application of the low frequency stimulation signal includes multiple stimulation cycles.

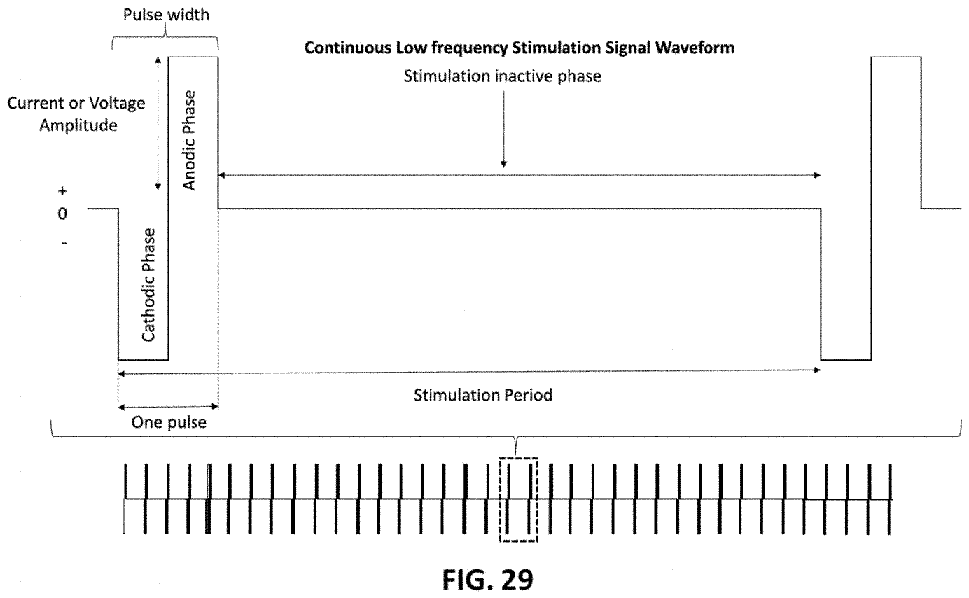

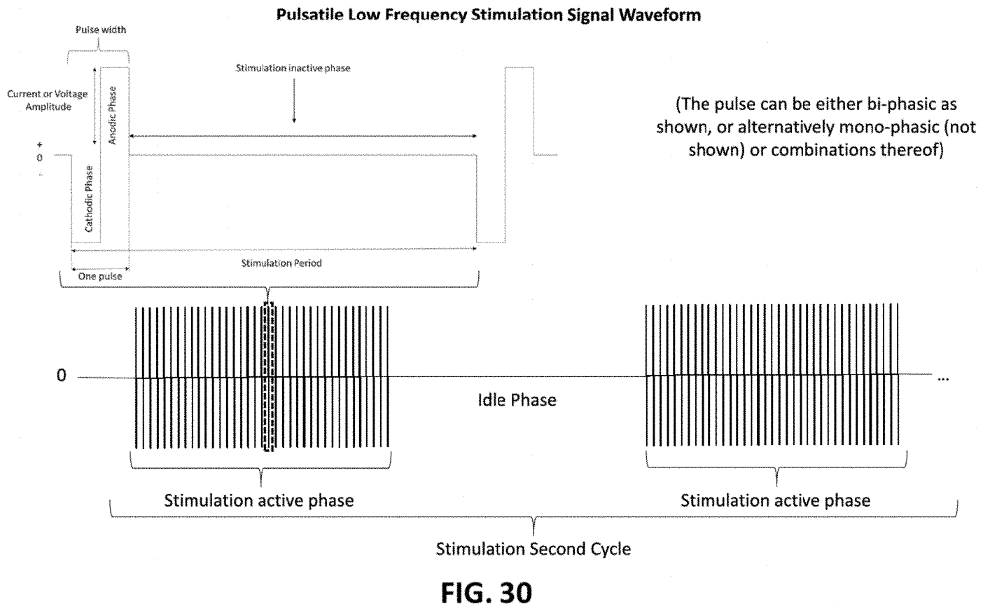

In embodiments, the low frequency stimulation signal is continuous, having multiple stimulation cycles with optional stimulation inactive phases but without idle phase. An example of continuous low frequency stimulation signal is shown in FIG. 29. In other embodiments, the low frequency stimulation signal is pulsatile, having multiple stimulation phases and at least one idle phase, each of the multiple stimulation phase comprising two or more stimulation cycles and optional stimulation inactive phases. An example of pulsatile low frequency stimulation signal is shown in FIG. 30.

In embodiments, a system and method of applying a low frequency stimulation signal having parameters that upregulate/stimulate nerve activity to a nerve in a subject comprises: applying a low frequency stimulation signal to a nerve or a nerve branch/fiber or an organ, wherein the low frequency stimulation signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. In embodiments, the stimulation signal further comprises at least one stimulation active phase, wherein each of the at least one stimulation active phase comprises two or more stimulation cycle, and wherein each of the at least one stimulation active phase is separated by an idle phase. In embodiments, the stimulation inactive phase can vary in time between each stimulation active phase.

In embodiments, the stimulation active phase is at least about 10 seconds. In embodiments, the stimulation active phase is about 10 seconds to about 30 minutes, about 10 seconds to about 25 minutes, about 10 seconds to about 20 minutes, about 10 seconds to about 15 minutes, about 10 seconds to about 10 minutes, about 10 seconds to about 5 minutes, about 10 seconds to about 1 minute, about 10 seconds to about 30 seconds, or about 20 seconds to about 30 minutes, about 30 seconds to about 30 minutes, about 40 seconds to about 30 minutes, about 50 seconds to about 30 minutes, about 1 minute to about 30 minutes, about 5 minutes to about 30 minutes, about 10 minutes to about 30 minutes, about 15 minutes to about 30 minutes, about 20 minutes to about 30 minutes, about 25 minutes to about 30 minutes.

In embodiments, the stimulation active phase comprises at least 2 to 100 stimulation cycles, at least 2 to 90, at least 2 to 80, at least 2 to 70, at least 2 to 60, at least 2 to 50, at least 2 to 40, at least 2 to 30, at least 2 to 20, at least 2 to 10, at least 2 to 5, or at least 2 to 4 stimulation cycles.

In embodiments, the idle phase is in a ratio to the stimulation active phase of about 200 to 1, 180 to 1, 140 to 1, 100 to 1, 60 to 1, 20 to 1, 10 to 1, 5 to 1, 1 to 1, 1 to 2, 1 to 5, 1 to 10, 1 to 201, 1 to 60, 1 to 100, 1 to 140, 1 to 180, or 1 to 200. In embodiments, the idle phase is at least 10 seconds. In embodiments, the idle phase is 10 seconds to 30 minutes, 10 seconds to about 30 minutes, about 10 seconds to about 25 minutes, about 10 seconds to about 20 minutes, about 10 seconds to about 15 minutes, about 10 seconds to about 10 minutes, about 10 seconds to about 5 minutes, about 10 seconds to about 1 minute, about 10 seconds to about 30 seconds, or about 20 seconds to about 30 minutes, about 30 seconds to about 30 minutes, about 40 seconds to about 30 minutes, about 50 seconds to about 30 minutes, about 1 minute to about 30 minutes, about 5 minutes to about 30 minutes, about 10 minutes to about 30 minutes, about 15 minutes to about 30 minutes, about 20 minutes to about 30 minutes, about 25 minutes to about 30 minutes.

An exemplary embodiment of pulsatile stimulation waveform is shown in FIG. 30. A stimulation cycle comprises at least one stimulation period; and a stimulation inactive phase. The pulsatile stimulation waveform comprises two or more stimulation active phase that includes more than one stimulation cycles and a stimulation inactive phase. Energy savings are realized by including stimulation inactive phases between the pulses as well as between stimulation inactive phases between stimulation active phases. In addition, the length of the stimulation inactive phases, stimulation inactive phases and/or the number of stimulation periods can be varied to provide application of a total amount of charge during an on time while varying the impact on the nerve. In embodiments, the frequency of the electrical signal treatment is selected to upregulate activity on the nerve and is at most 199 Hz, at most 150 Hz, at most 100 Hz, at most 50 Hz, at most 40 Hz, at most 30 Hz, at most 20 Hz, at most 10 Hz, at most 1 Hz, at most 0.5 Hz, at most 0.1 Hz, at most 0.05 Hz, or at most 0.01 Hz or less.

In yet other embodiments, a system and method of applying a low frequency stimulation signal having parameters to upregulate/stimulate nerve activity to a nerve in a subject comprises: applying the a low frequency stimulation signal to the nerve during an on time, wherein the low frequency stimulation signal comprises a first pattern and a second pattern which differ from one another. In embodiments, the first pattern comprises at least one stimulation cycle. In other embodiments, the first pattern comprises more than one stimulation active phase, wherein each stimulation active phase comprises more than one stimulation cycle, and each stimulation active phase is separated by an idle phase. In embodiments, the second pattern comprises at least one stimulation cycle. In embodiments, the second pattern comprises more than one stimulation active phase, wherein each stimulation active phase comprises more than one stimulation cycle, and each stimulation active phase is separated by an idle phase.

In yet other embodiments, a system and method of applying an low frequency stimulation signal having parameters to upregulate/stimulate nerve activity to a nerve in a subject comprises: applying the low frequency stimulation signal to the nerve during an on time, wherein the electrical signal comprises a first pattern comprising at least one stimulation cycle; and a second pattern comprising more than one stimulation active phase, wherein each stimulation active phase comprises more than one stimulation cycle, and each stimulation active phase is separated by an idle phase, wherein the first and second patterns have a different amplitude and/or different on times. In embodiments, the stimulation cycle comprises at least one stimulation period and a stimulation inactive phase, each of the at least one stimulation period comprising a pulse and optionally, a pulse delay, wherein each stimulation period has a frequency of about 0.01 Hz to 199 Hz. In embodiments, the first pattern has an amplitude greater than the second pattern. In embodiments, the ratio of the amplitude of the first pattern to the amplitude of the second pattern is at least 10 to 1, 8 to 1, 6 to 1, 4 to 1, 2 to 1 or 4 to 3. In embodiments, the amplitude of the first embodiment is maintained or held constant during the time the first pattern is applied. In other embodiments, the amplitude of the second embodiment, while different than the first pattern, is maintained or held constant during the time period the second pattern of electrical signal is applied.

In embodiments, the low frequency stimulation signal has a frequency of a period which comprises a cathodic and/or anodic phase and may have pulse delays, wherein the frequency is at most 199 Hz, at most 150 Hz, at most 100 Hz, at most 50 Hz, at most 40 Hz, at most 30 Hz, at most 20 Hz, at most 10 Hz, at most 1 Hz, at most 0.5 Hz, at most 0.1 Hz, at most 0.05 Hz, or at most 0.01 Hz or less. In other embodiments, the frequencies range 0.01 Hz to 199 Hz, or from about 0.01 Hz to about 100 Hz, or from about 0.01 Hz to about 50 Hz, or from about 0.01 Hz to about 30 Hz, or from about 0.01 Hz to about 10 Hz. In embodiments, signals at such frequencies can upregulate or stimulate nerve activity.

In embodiments, the amplitude of the signal is at least 0.01 mAmp. In other embodiments, the amplitude ranges from about 0.01 to 20 mAmps, 0.01 to 15 mAmps, 0.01 to 10 mAmps, 0.01 to 8 mAmps, or 0.01 to 5 mAmps.

In embodiments, the amplitude is at least 0.01 volt. In other embodiments, the amplitude ranges from about 0.01 to 20 volts, 0.01 to 15 volts, 0.01 to 10 volts, 0.01 to 8 volts, or 0.01 to 5 volts.

In embodiments, the on time is at least about 30 seconds. In other embodiments, the on time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow pulsatile stimulation of the nerve with improved efficiency and reduced energy consumption. In embodiments, the off time may be minimized due to the presence of stimulation inactive phases and/or idle phases. In embodiments, off times are at least about 30 seconds. In other embodiments, the off time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include off times of varying amounts. For example, a therapy cycle can include 1 minute of on time, 1 minute of off time, 2 minutes of on time, followed by 5 minutes of off time.

In any of the systems and methods described herein, application of an electrical signal can be initiated or terminated using a ramp up and/or ramp down of amplitude and/or pulse width and/or frequency. In embodiments, such ramp up and ramp down times are useful to minimize sensations or discomfort from application of an electrical signal to a nerve and/or to favorably change the kinetics of hormone secretion (e.g., insulin release). In embodiments, a ramp up includes multiple pulses, each pulse has an increasing increment of amplitude and/or an increasing increment of pulse width and/or a decreasing increment of frequency. In embodiments, a ramp down includes multiple pulses, each pulse has a decreasing increment of amplitude and/or a decreasing increment of pulse width and/or a decreasing increment of frequency.

The amplitudes can range from about 0.01 to 20 mA, 0.01 to 20 mAmps, 0.01 to 15 mAmps, 0.01 to 10 mAmps, 0.01 to 8 mAmps, or 0.1 to 5 mAmps. In a ramp up, the initial amplitude can be higher than 0.01, for example starting at 3 mAmps. In a ramp down, the initial amplitude can be lower than 20, for example starting at 10 mAmps. In ramp up and/or ramp down the amplitude is changing during the ramp up or down time, whereas in the other methods described herein once a specific amplitude is reached it is maintained for the duration of the first pattern, followed by change to the second amplitude which is then maintained for the duration of the second pattern. In some embodiments, the time period for a ramp up and/or ramp down is about 10 seconds to about 15 minutes. In some embodiments, a ramping up of the amplitude occupies all of the time of the on time of a first and/or second pattern of the low frequency stimulation signal.

In some embodiments, a system and method of independently and separately applying multiple electrical signals to multiple nerves or nerve branches/fibers in one subject. The electrical signals can be any of the high frequency signals, the low frequency stimulation signals, or other signals as described in the present disclosure. In embodiments, the method and system comprises independently and separately applying a high frequency signal having parameters to downregulate/block nerve activity to a nerve or a nerve branch/fiber or an organ in a subject and applying a low frequency stimulation signal having parameters to upregulate/stimulate nerve activity to a separate nerve or a separate nerve branch/fiber or a separate organ in the same subject. In embodiments, the high frequency signal and the low frequency stimulation signal are applied simultaneously or concurrently. In embodiments, the high frequency signal and the low frequency stimulation signal are applied at different/separate times, for example, in treating hypoglycemia. In embodiments, the high frequency signal and the low frequency stimulation signal are applied in a coordinately fashion to maximize therapeutic effect.

3. Duty Cycle

A duty cycle is measured by the percentage of time when charge is being delivered to the nerve during one cycle. In high frequency signals, one cycle includes either a microsecond cycle, a millisecond cycle, or both. In low frequency signals, one cycle includes either a stimulation cycle, or a Stimulation Second Cycle of pulsatile stimulation waveform as shown in FIG. 30. The duty cycle according to the present disclosure relates to both high frequency signals and low frequency stimulation signals, but is particularly useful in characterizing high frequency signals.

In high frequency signals, an alternative way to characterize the addition of microsecond and/or millisecond inactive phases is to characterize the addition as a change in a duty cycle. A duty cycle is measured by the percentage of time charge is being delivered to the nerve during one cycle, including either a microsecond cycle, a millisecond cycle, or both. A cycle can also be the length of an on time as in FIGS. 10 and 11. If a signal is being delivered to the nerve with no microsecond inactive phases, pulse delays, or millisecond inactive phases the duty cycle is characterized as 100%. To determine the percentage of the duty cycle being applied during an on time, the pulse widths of the charge and recharge phase of a cycle during an on time (not including any pulse delays) are added and divided by total time of the microsecond and/or millisecond cycle.

In an embodiment, a HFAC/HFAV low duty cycle is illustrated by FIG. 6. If, for example, the pulse width in FIG. 6 is 200 microseconds for each charge and recharge phase, and the microsecond inactive phase is 1600 microseconds the duty cycle can be calculated. The microsecond cycle comprises 400 microseconds of a charge and recharge phase, followed by an inactive phase of 1600 microseconds for a total of 2000 microseconds. The cycle repeats itself for the duration of the on time. The duty cycle is calculated:

(400 microseconds/2000 microseconds)×100=20 percent

This decrease in duty cycle due to microsecond inactive phases is as compared to 100% as shown in FIG. 5.

Yet another embodiment of a HFAC/HFAV low duty cycle with microsecond cycles and pulse delays is illustrated in FIG. 7. As an example, the pulse width is 70 microseconds with 30 microsecond pulse delays between the charge and recharge phase and following the recharge phase. The total time period of the charge recharge phase and pulse delays is 200 microseconds making the frequency 5000 Hz. There is a microsecond inactive phase between the charge/recharge phases of 20 microseconds. During the microsecond cycle charge is delivered for 140 microseconds. The microsecond cycle is 220 microseconds long. The duty cycle is (140 microseconds/220 microseconds)×100=64%.

Another embodiment of a HFAC/HFAV low duty cycle with microsecond cycles forming a millisecond active phase followed by a millisecond inactive phase is illustrated in FIG. 8. As an example of this electrical signal pattern, on the millisecond scale, one repetitive cycle is 60 milliseconds long including a millisecond inactive phase of 20 milliseconds and a millisecond active phase of 40 milliseconds. Each millisecond active phase includes 40 microsecond cycles. Turning to the microsecond cycle, this example has a pulse width of 100 microseconds with one charge and one recharge cycle (making period 200 microseconds and the frequency 5000 Hz) followed by a 800 microsecond inactive phase. For 1000 microseconds charge is being delivered for 200 microseconds. This repeats itself 40 times before the 20 millisecond inactive phase. The amount of time charge is being delivered during the 60 millisecond repetitive cycle is 200 microseconds×40 (total microsecond cycles)=8000 microseconds. Thus, the duty cycle is (8000 microseconds/60 milliseconds)×100=13.3%.

Another embodiment of a HFAC/HFAV low duty cycle with microsecond cycles forming millisecond active phases followed by millisecond inactive phases is illustrated in FIG. 9. On the microsecond scale, the pulse width is 70 microseconds with 30 microsecond pulse delays between the charge and recharge phase and following the recharge phase. The period is 200 microseconds making the frequency 5000 Hz. There is a microsecond inactive phase between the charge/recharge phases of 20 microseconds. The microsecond cycle is 220 microseconds long. The microsecond cycles form a 70.4 millisecond active phase followed by a 29.6 millisecond inactive phase. In the 70.4 millisecond active phase there are 70.4/0.22=320 microsecond cycles. Each microsecond cycle is delivering charge for 140 microseconds. For each millisecond active phase charge is delivered for 140 microseconds×320 microsecond cycles=44,800 microseconds. One repetitive cycle is 100 milliseconds long so the duty cycle is (44,800 microseconds/100 milliseconds)×100=44.8%.

Yet another embodiment of an HFAC/HFAV low duty cycle is illustrated in FIG. 10. The total on time is 120 seconds. As an example, the first pattern is delivered for 30 seconds. The first pattern comprises more than one microsecond cycle, where the pulse amplitude is delivered at first amplitude for 30 seconds, followed by the second pattern of 90 seconds at a second amplitude. The second pattern comprises microsecond cycles which form millisecond active phases followed by millisecond inactive phases with the pulse amplitude reduced 25%. For the first pattern, the microsecond cycles are 1000 microseconds long with a 100 microsecond pulse width for each charge and recharge phase (making the period 200 microseconds and the frequency 5000 Hz) and an 800 microsecond inactive phase. For the second pattern the microsecond cycles are 1000 microseconds long with a 100 microsecond pulse width, one charge and discharge phase (making the period 200 microseconds and the frequency 5000 Hz) and an 800 microsecond inactive phase and form millisecond active phases of 40 milliseconds long followed by a 20 millisecond inactive phases.

The total time charge is being delivered can be broken into two parts and then added together for the example given for FIG. 10. For the first 30 seconds the total time charge is being delivered is calculated as such: for every 1000 microseconds, 200 microseconds of charge is being delivered. This micro repetitive pattern occurs for 30 seconds. Thus, the time that charge is being delivered for the first 30 seconds is 200 (microseconds)/1000 microseconds×30 (seconds)=6 seconds. Calculating the time charge is being delivered for the next 90 seconds is as follows: here there are repetitive phases on the millisecond time scale (60 milliseconds) and on the microsecond time scale (1000 microseconds). On the microsecond time scale the active phase is 200 microseconds long followed by an 800 microseconds inactive phase. This repeats itself 60 times every 60 milliseconds. Thus, for 60 milliseconds the amount of time charge is being delivered is 200 (microseconds)×60 (active phases)=12,000 microseconds (or 12 milliseconds). In 90 seconds, there are 90×60 milliseconds=1500 of these 60 millisecond repetitive cycles. The total time charge is being delivered for the last 90 seconds of this second pattern would then be 12 milliseconds×1500=18 seconds. For the total 120 seconds of this algorithm the duty cycle would be ((6 seconds+18 seconds)/120 seconds)×100=16.7%.

It should be noted that lowering the duty cycle decreases the amount of energy delivered, but in addition to this, lowering the current amplitude for the last 90 seconds decreases the amount of energy delivered even more.

Decreasing the duty cycle using microsecond and/or millisecond inactive phases results in downregulating activity on the nerve while minimizing the energy requirements needed to downregulate the nerve and maintain down regulation during an on time.

In embodiments, a duty cycle including at least one microsecond and/or millisecond inactive phase is a low duty cycle of about 75% or less, 70% or less, 60% or less, 50% or less, 40% or less, 30% or less, 20% or less, and 10% or less.

In some embodiments, the disclosure provides a low duty cycle high frequency alternating current (HFAC) signal algorithm by utilizing a pulse width that is shorter than the period of the signal. The period of the signal is the length of time of one charge phase and one recharge phase, which can include one or more pulse delays. A shorter pulse width equates with a lower duty cycle and greater energy efficiency is realized. A lower duty cycle can be utilized for frequencies of about 200 Hz to about 25 kHz. The pulse width for a biphasic signal that has a 100% duty cycle for a given frequency is 1 divided by the frequency and further divided by 2.

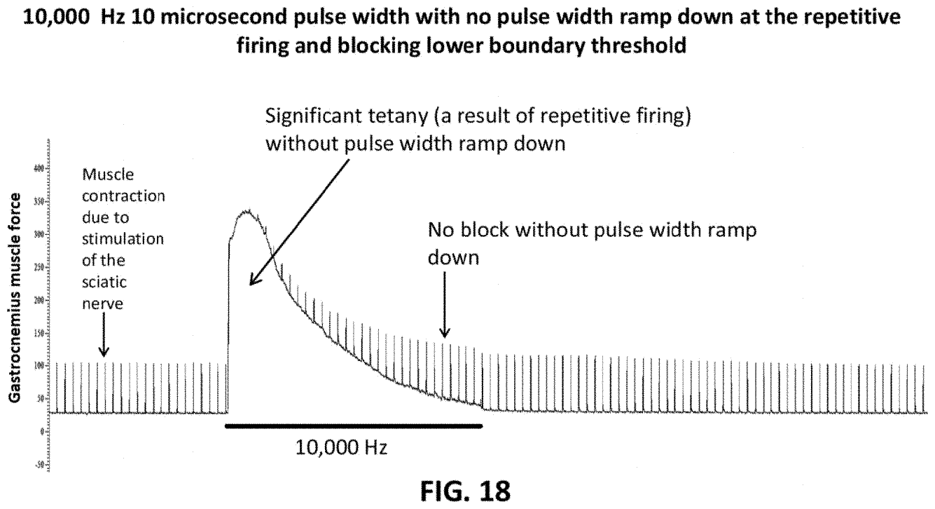

In embodiments, the pulse width is selected to be above a lower boundary threshold. While not mean to limit the invention, it is believed that an undesirable end organ response can occur when the duty cycle reaches a lower boundary threshold pulse width. This lower boundary threshold pulse width is substantially below the pulse width for a selected blocking frequency at a 100% duty cycle and is one at which no blocking of the nerve is observed or expected and/or at which repetitive firing is observed. The lower boundary threshold can be determined by applying HFAC for a period of time at a pulse width that is substantially shorter than a pulse width that is 100% of the duty cycle (e.g. 10% duty cycle). An example of this would be a pulse width of 10 microseconds at a frequency of 10,000 Hz with no pulse ramp down (FIG. 18). As shown in FIG. 18, at a pulse width of 10 microseconds, repetitive firing and tetany is observed, and no block of nerve conduction is seen. This profile represents a pulse width at or below a lower boundary threshold.

In embodiments, a lower boundary threshold can be determined by application of a variety of pulse widths without any pulse width ramp down or up and determining whether the patient feels a sensation. Pulse widths resulting in a sensation are at or below a boundary threshold for that frequency. For example, a pulse width is selected for a given frequency at a 10% duty cycle and applied to a patient to determine if the patient feels a sensation. If a sensation is felt, HFAC delivery is then stopped for a period of time to allow patient recovery and applied again at the same frequency as the first application but at a longer pulse width (e.g. 1-10 microseconds longer) than the first application. This would be repeated until the patient does not feel sensations. The pulse width at which the patient no longer feels sensations is above the lower boundary threshold. If at the first application, the patient does not feel sensations the same process would be conducted but the pulse widths would be decreased between each HFAC application. The lower boundary threshold or a pulse width that is at or below the lower boundary threshold would be determined by the pulse width in which the patient first experiences sensations.

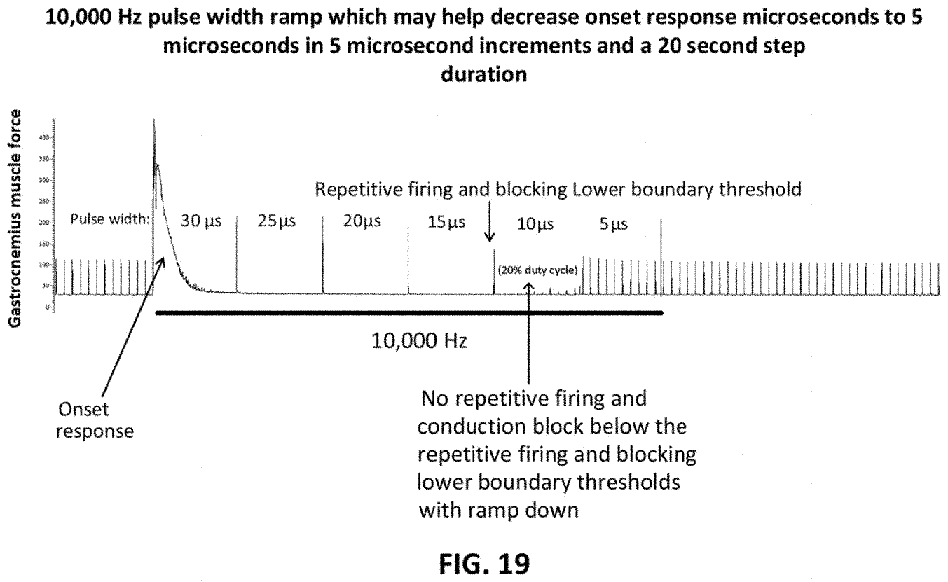

In embodiments, employing a pulse width ramp down or ramp up provides for nerve conduction block pulse width at or below a lower boundary threshold. (FIG. 19) Pulse widths with no pulse width ramp down below the blocking lower boundary threshold do not induce conduction block. Going below this boundary and inducing conduction block can be achieved by starting at a pulse width above (e.g. at least 1% longer than the lower boundary threshold up to 100% of the duty cycle) the boundary threshold and ramping down the pulse width until a constant pulse width is reached below the boundary threshold. In embodiments, the ramp down occurs in steps with a duration of about 1 second to 60 seconds at a rate that is linear or non-linear. In other embodiments, the pulse width ramp down could also be continuous which means each successive pulse has a decreased pulse width, and the rate of the continuous pulse width ramp down can be linear or non-linear. In embodiments, the incremental decrease in pulse width would be around 1 to 10 microseconds. In yet other embodiments, during the pulse width ramp down or ramp up the current amplitude (or voltage) can either be increased or decreased. In embodiments, during the pulse width ramp down the time between pulses can either be increased or decreased at a linear or non-linear rate. In other embodiments, during the ramp down the duty cycle can be fixed, increased or decreased.

FIG. 19 is an example of ramping down pulse width to a pulse width below the boundary threshold. For example, at a frequency of 10,000 Hz and an initial pulse width of 30 microseconds the pulse width is decreased to 25 microseconds for 20 seconds. Next the pulse width would decrease to 20 microseconds for 20 seconds and next to 15 microseconds for 20 seconds and follow the same pattern until the pulse width reaches 5 microseconds and is constant for the duration of the on time. Blocking of the nerve occurs at pulse width of 5 microseconds at a frequency of 10,000 Hz with a current amplitude of 0.1 mA to 20 mA depending on electrode placement and impedance.

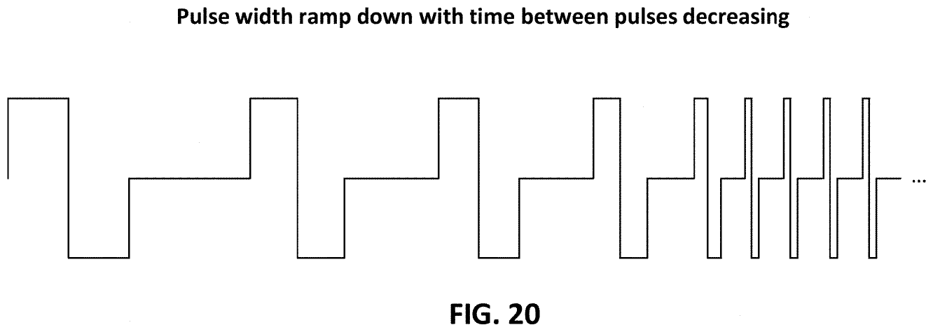

In some embodiments, a pulse width ramp down or ramp up can vary not only in the pulse width but also in the time between pulses. For example, FIG. 20 shows a pulse width ramp down with time between pulses decreasing. In embodiments, decreasing the time between pulses and the pulse width duration at the same time the duty cycle remains constant. In other embodiments, in a pulse width ramp up the time between the pulses can be increasing at the same time and the duty cycle would remain constant.

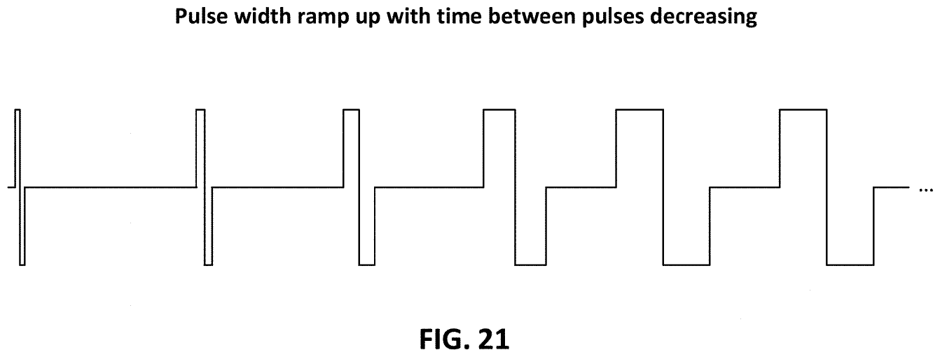

In some embodiments, ramping up of pulse widths is desired (see FIG. 21). In this embodiment, the pulse width at the start of HFAC delivery would be lower than the lower boundary threshold. Starting with pulse widths lower than the lower boundary threshold and ramping up pulse width durations may eliminate the repetitive firing. In embodiments, the ramp up can occur in steps with a duration of about 1 second to 60 seconds at a rate that is linear or non-linear. In other embodiments, the pulse width ramp up could also be continuous which means each successive pulse has an increased pulse width. The rate of the continuous pulse width ramp up can be linear or non-linear. In embodiments, the incremental increase in pulse width would be around 1 to 10 microseconds. In embodiments, during the pulse width ramp up the current amplitude (or voltage) can either be increased or decreased. In other embodiments, during the pulse width ramp up the time between pulses can either be increased or decreased at a linear or non-linear rate. During the ramp up the duty cycle can be fixed, increased or decreased. To eliminate nerve activity, and undesirable sensations, during or at the initiation of a high frequency alternating current conduction block (HFAC), a pulse width ramp down can be used in combination with a current (or voltage) ramp down. Initiation of HFAC with an amplitude that is substantially (about 5 times) above a blocking threshold may eliminate an onset response. A blocking threshold is a current (or voltage) amplitude in which conduction block is realized with a HFAC signal at or above the current (or voltage) threshold and no blockade (or a partial block) occurs below this amplitude. The power consumption of a HFAC pulse generator is considerable and a sustained current (or voltage) output that is substantially greater than the blocking threshold would not be desirable. Initiation of HFAC with a considerably high current (or voltage) amplitude and decreasing the level, in a linear or non-linear rate, may avoid an onset response and sustain blockade when the current (or voltage) amplitude is lowered to the blocking threshold.

Lower energy consumption can also be realized by a low duty cycle in which the pulse width of the HFAC signal is substantially lower than half of the period of the signal. However, sustained repetitive firing of action potentials for the duration of the signal and non-realization of conduction block may occur at short pulse widths (below the lower boundary pulse width threshold, FIG. 18). The probability of these unwanted effects decreases at pulse widths that are half (100% duty cycle), or close to half (approximately 90% duty cycle), of the duration of the period of the HFAC signal. Initiation of HFAC with pulse widths at or close to a 100% duty and ramping down the duration of the pulse width to a low duty cycle, below the lower boundary threshold, may eliminate continuous repetitive firing of action potentials for the duration of the HFAC signal and un-realized blockade (FIG. 19). With this method energy savings would be realized without the aforementioned unwanted side effects.

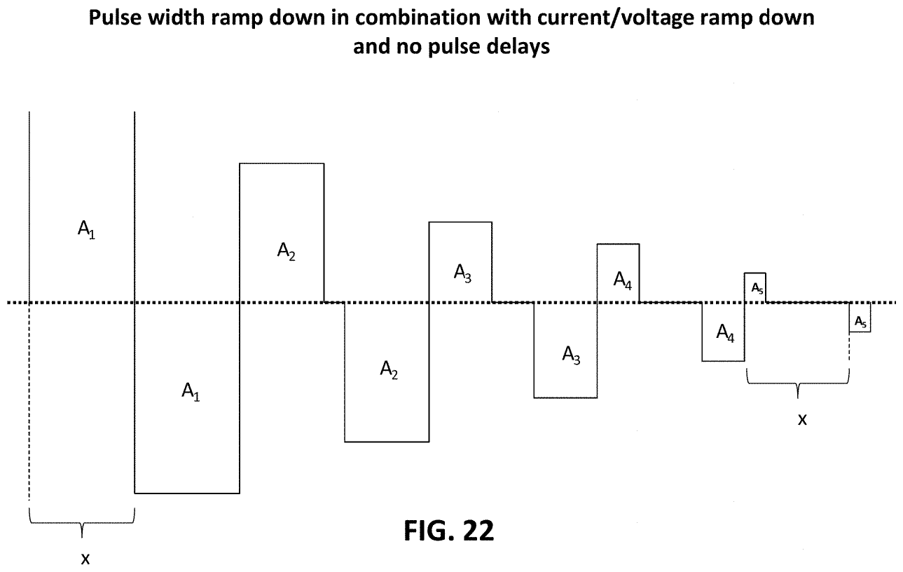

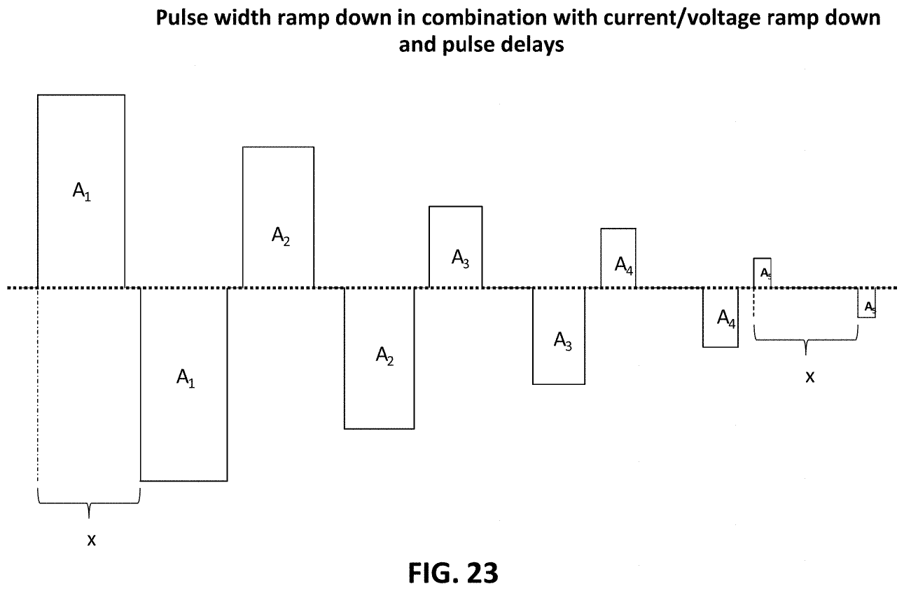

A combination of current (or voltage) ramp down with a concurrent pulse width ramp down (FIGS. 22 and 23) would decrease repetitive firing at the onset of block as well as during the course of the block with using a low duty cycle signal. Ax indicates the area of the charge and recharge phases. The area of the charge phase equals the area of the recharge phase for each cycle to avoid a direct current offset. The areas progressively decrease due to the decrease in pulse width in combination with a decrease in amplitude. X depicts that the time from the start of the charge phase to the start of the recharge phase remains constant. However, this can vary in a linear or non-linear rate. The current (or voltage) ramp down could occur continuously with the pulse width ramp down. The current (or voltage) ramp down could precede or follow the pulse width ramp down. The rate of the current (or voltage) ramp down could be the same or different than the pulse width ramp downs. The rates of the current (or voltage) ramp downs may be linear or non-linear or switch from linearity to non-linearity during the ramps, or vice versa. In other instances a current (or voltage) ramp down could occur with a fixed low duty cycle HFAC signal. In other instances the pulse width ramp down could occur with a fixed current (or voltage).

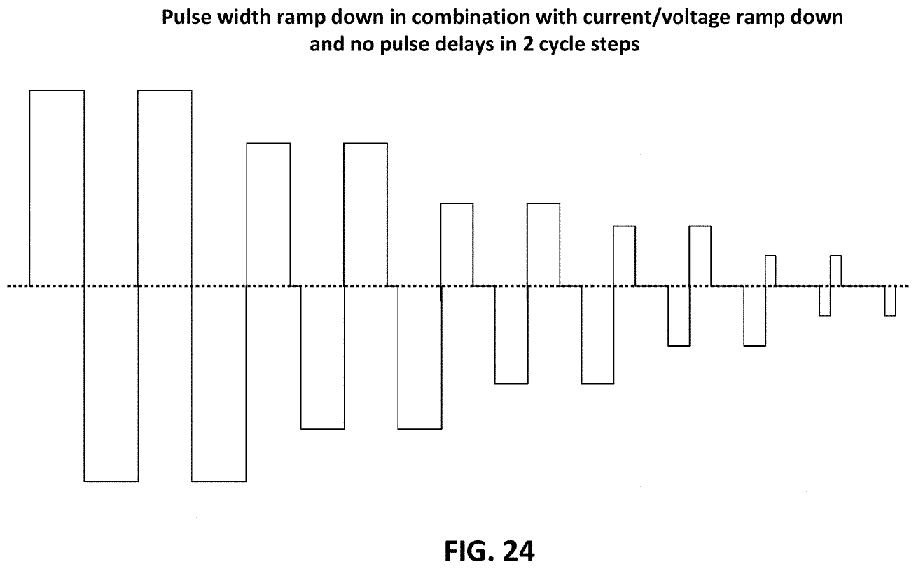

The current (or voltage) and/or pulse width ramp down can be continuous or occur in steps (FIG. 24). FIG. 24 describes a pulse width ramp down in combination with current/voltage ramp down and no pulse delays in 2 cycle steps. Cycles per steps can range up to the number of cycles to fill an about 5 min period. Steps for the pulse width ramp down and/or current (or voltage) ramp down could be as low as two cycles or as many cycles that fill about 5 minutes. Steps for the duration of the pulse widths can be 1% to 99% of the initial pulse width. The steps for the current (or voltage) ramp down can be from 0.1 mA (or volts) to 19.9 mA (or volts). For each cycle the area of the charge and recharge phase are the same (FIGS. 22 and 23). The entire time of the current (or voltage) and/or pulse width ramps can range from about 5 seconds to 30 minutes. Current amplitudes at the initiation of the ramp down can range from about 0.2 mA to about 20 mA. Voltage amplitudes can range from about 0.2 volts to about 20 volts. Frequencies can range from about 200 Hz to 100 kHz.

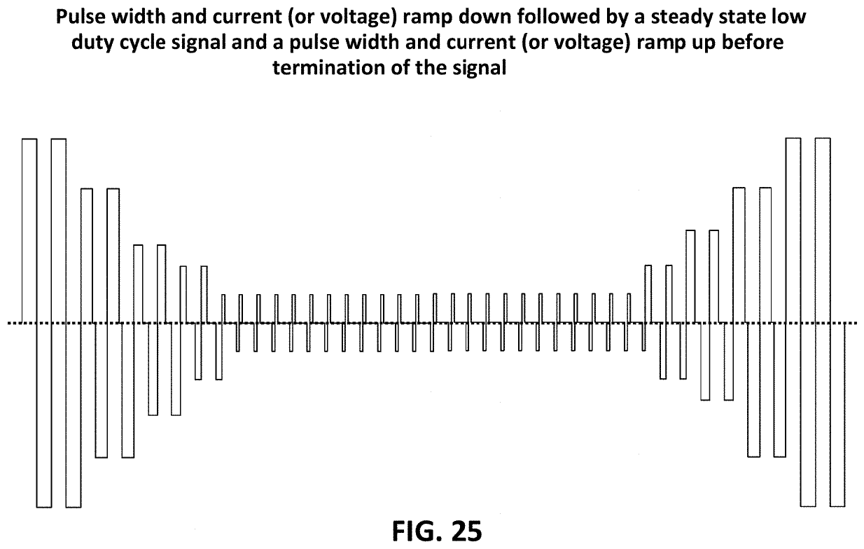

Nerve activity can occur at the termination of HFAC. To avoid this a ramp up of current (or voltage) and/or pulse width ramp up can occur prior to the cessation of 5000 Hz (FIG. 25). The ramps can occur about 5 second to 30 min prior to the termination of the HFAC signal. The rate of the ramp ups can be linear or non-linear or switch from linearity to non-linearity, or vice versa, during the ramp. The voltage and current ramps can be concurrent or non-concurrent. For example the voltage ramp can precede or follow the pulse width ramp. The ramp up can be continuous or occur in steps. Steps for the pulse width ramp up and/or current (or voltage) ramp up could be as low as two cycles or as many cycles that fill about 5 minutes. Steps for the duration of the pulse widths can be 1% of the duty cycle to 99% of the final pulse width. The steps for the current (or voltage) ramp up can be from 0.1 mA (or volts) to 19.9 mA (or volts). For each cycle the area of the charge and recharge phase are the same. In other instances a current (or voltage) ramp up could occur with a fixed duty cycle HFAC signal. In other instances the pulse width ramp up could occur with a fixed current (or voltage).

4. Therapy Programs

The external charger 101 and/or the neuroregulator 104, 104′ contain software to permit use of the therapy system 100 in a programmable variety of therapy schedules, electrical signal delivery, therapy programs, operational modes, system monitoring and interfaces as will be described herein.

In embodiments, system software can be stored on a variety of computer devices, such as an external smartphone or tablet, external programmer, the neuroregulator, and/or external charger.

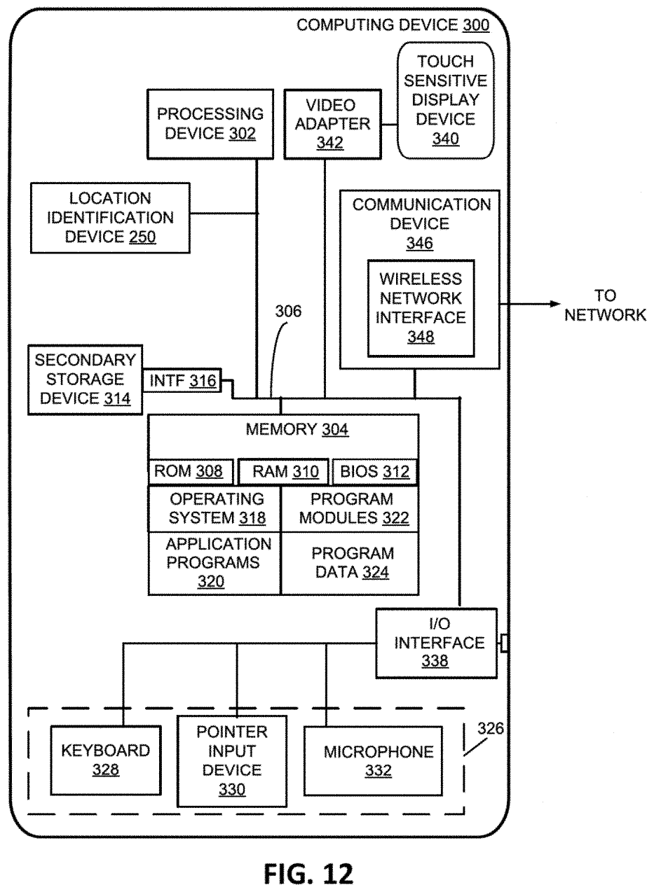

Referring to FIG. 12, an exemplary architecture of a computing device that can be used to implement aspects of the present disclosure is illustrated. For example, the external charger 101, the neuroregulator 104, 104′, an external programmer, an external smartphone of tablet, or various systems and devices of the therapy system 100 can be implemented with at least some of the components of the computing device as illustrated in FIG. 12. Such a computing device is designated herein as reference numeral 300. The computing device 300 is used to execute the operating system, application programs, and software modules (including the software engines) described herein.

The computing device 300 includes, in some embodiments, at least one processing device 302, such as a central processing unit (CPU). A variety of processing devices are available from a variety of manufacturers, for example, Intel or Advanced Micro Devices. In this example, the computing device 300 also includes a system memory 304, and a system bus 306 that couples various system components including the system memory 304 to the processing device 302. The system bus 306 is one of any number of types of bus structures including a memory bus, or memory controller; a peripheral bus; and a local bus using any of a variety of bus architectures.

Examples of computing devices suitable for the computing device 300 include a desktop computer, a laptop computer, a tablet computer, a mobile device (such as a smart phone, an iPod® mobile digital device, or other mobile devices), or other devices configured to process digital instructions.

The system memory 304 includes read only memory 308 and random access memory 310. A basic input/output system 312 containing the basic routines that act to transfer information within computing device 300, such as during start up, is typically stored in the read only memory 308.

The computing device 300 also includes a secondary storage device 314 in some embodiments, such as a hard disk drive, for storing digital data. The secondary storage device 314 is connected to the system bus 306 by a secondary storage interface 316. The secondary storage devices and their associated computer readable media provide nonvolatile storage of computer readable instructions (including application programs and program modules), data structures, and other data for the computing device 300.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 314 or memory 304, including an operating system 318, one or more application programs 320, other program modules 322, and program data 324.

In some embodiments, computing device 300 includes input devices to enable a user to provide inputs to the computing device 300. Examples of input devices 326 include a keyboard 328, pointer input device 330, microphone 332, and touch sensitive display 340. Other embodiments include other input devices 326. The input devices are often connected to the processing device 302 through an input/output interface 338 that is coupled to the system bus 306. These input devices 326 can be connected by any number of input/output interfaces, such as a parallel port, serial port, game port, or a universal serial bus. Wireless communication between input devices and interface 338 is possible as well, and includes infrared, BLUETOOTH® wireless technology, WiFi technology (802.11a/b/g/n etc.), cellular, or other radio frequency communication systems in some possible embodiments.

In this example embodiment, a touch sensitive display device 340 is also connected to the system bus 306 via an interface, such as a video adapter 342. The touch sensitive display device 340 includes touch sensors for receiving input from a user when the user touches the display. Such sensors can be capacitive sensors, pressure sensors, or other touch sensors. The sensors not only detect contact with the display, but also the location of the contact and movement of the contact over time. For example, a user can move a finger or stylus across the screen to provide written inputs. The written inputs are evaluated and, in some embodiments, converted into text inputs.

In addition to the display device 340, the computing device 300 can include various other peripheral devices (not shown), such as speakers or a printer.

The computing device 300 further includes a communication device 346 configured to establish communication across the network. In some embodiments, when used in a local area networking environment or a wide area networking environment (such as the Internet), the computing device 300 is typically connected to the network through a network interface, such as a wireless network interface 348. Other possible embodiments use other wired and/or wireless communication devices. For example, some embodiments of the computing device 300 include an Ethernet network interface, or a modem for communicating across the network. In yet other embodiments, the communication device 346 is capable of short-range wireless communication. Short-range wireless communication is one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, and a Wi-Fi technology.

The computing device 300 typically includes at least some form of computer-readable media. Computer readable media includes any available media that can be accessed by the computing device 300. By way of example, computer-readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 300.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

As described above, the computing device typically includes at least some form of computer-readable media. Computer readable media includes any available media that can be accessed by the computing device. By way of example, computer-readable media include computer readable storage media and computer readable communication media.

The computer implemented methods as described herein are implemented by storing a series of instructions on the neuroregulator, external programmer, and/or the external charger. In embodiments, a user may select parameters of the electrical signal therapy and upon selection, selects a combination of electrical signal treatments including at least one micro second cycle, and/or at least one millisecond cycle and/or at least one millisecond inactive phase.

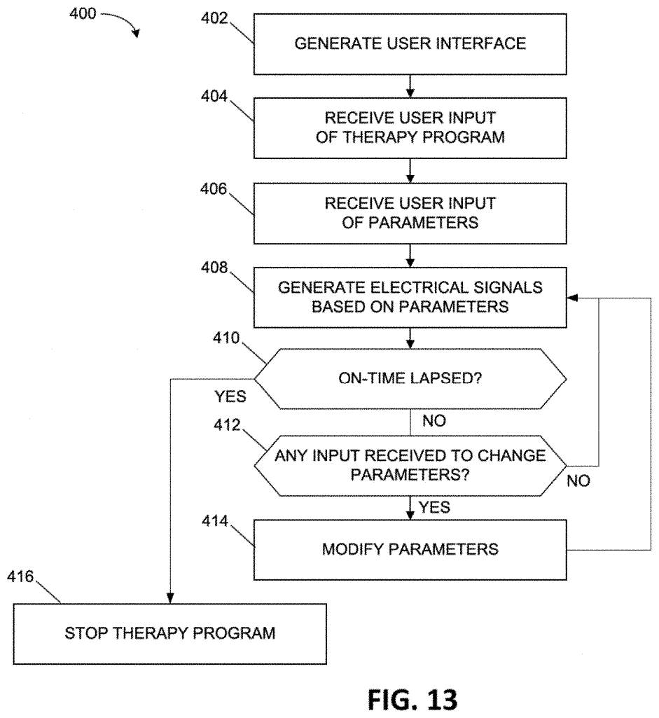

Referring to FIG. 13, an example method 400 of operating the therapy system 100 is illustrated. At operation 402, the system 100 generates a user interface configured to receive various inputs from a user, such as one or more parameters, therapy programs, schedules, and any other information usable for system operation. At operation 404, the system 100 receives a user input of a therapy program via the user interface. As described herein, the system 100 is configured to provide a plurality of therapy programs, and the user can select one of the therapy programs available through the user interface. At operation 406, the system 100 receives a user input of one or more parameters that determine the characteristics of a therapy program.

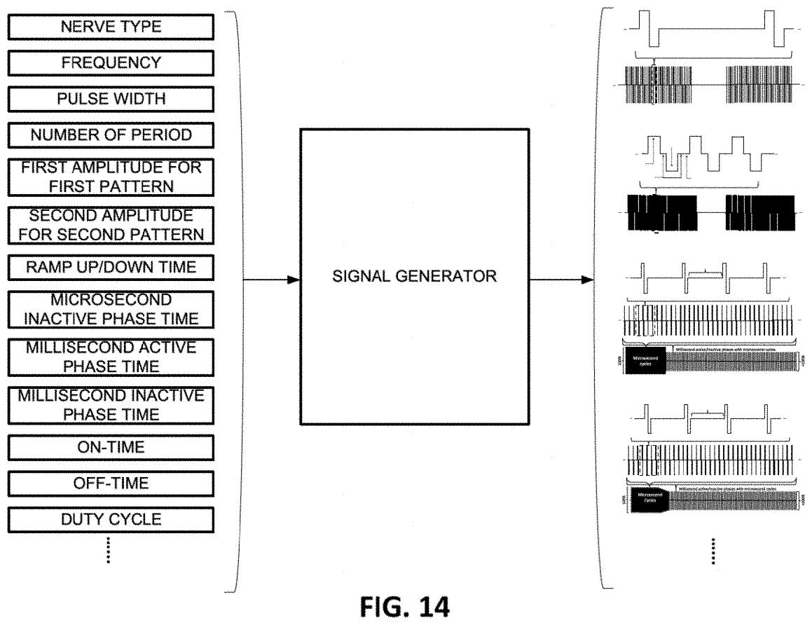

Examples of the parameters are described with reference to FIG. 14. At operation 408, the system 100 generates electrical signals based on the selected parameters, which implement the therapy program selected by the user. At operation 410, it is determined whether the on-time has lapsed. If so (“YES” at the operation 410), the system 100 stops the therapy program. If not (“NO” at the operation 410), the system 100 determines if there is any input for changing one or more of the parameters, at operation 412. If so (“YES” at the operation 412), the system 100 modifies the parameters based on the input, and continues the operation 408 and the subsequent operations. If not (“NO” at the operation 412), the system 100 continues the operation 408 and the subsequent operations.

As illustrated in FIG. 14, the system 100 receives and utilizes a plurality of parameters to generate various patterns of electrical signals for different therapy programs. Examples of the parameters are described as follows:

Parameters that are selected by a user include type of nerve. In embodiments, the type of nerve is selected from vagus nerve, renal nerve, renal artery, sympathetic nerves, and glossopharyngeal nerve.

In embodiments, a user can select parameters that feature a high frequency signal or a high frequency low duty cycle signal for downregulating/blocking nerve activity. A user can also select parameters that feature a low frequency stimulation signal for upregulating/stimulating nerve activity. A user can select parameters to independently and separately apply multiple electrical signals applied to multiple nerves or nerve branches/fibers. A user can also select parameters to concurrently or simultaneously apply multiple electrical signals applied to multiple nerves or nerve branches/fibers, or otherwise apply the multiple signals in a coordinated fashion.

In embodiments, a user selects a frequency of at least 200 Hz. In embodiments, the frequency choices in each period of a microsecond cycle are at least 200 Hz, at least 250 Hz, at least 300 Hz, at least 400 Hz, at least 500 Hz, at least 1000 Hz, at least 2000 Hz, at least 3000 Hz, at least 4000 Hz, or at least 5000 Hz, or at least 10.00 Hz, or at least 20,000 Hz, or at least 30,000 Hz, or at least 40,000 Hz, or at least 50,000 Hz, or at least 60,000 Hz, or at least 70,000 Hz, or at least 80,000 Hz, or at least 90,000 Hz, or at least 100,000 Hz, or at least 150 kHz, or at least 200 kHz, or at least 250 kHz or more. In other embodiments, the frequencies range from about 200 Hz to 250 kHz, 200 Hz to 200 kHz, 200 Hz to 150 kHz, 200 Hz to 100 kHz, 200 to 50 kHz, 200 Hz to 25 k Hz, 200 Hz to 10 kHz, or 200 Hz to 5000 Hz, or 200 Hz to 3000 Hz, or 200 Hz to 1500 Hz, or 200 to 1000 Hz. In other embodiments, the frequencies range from about 1000 Hz to 250 kHz, 1000 Hz to 200 kHz, 1000 Hz to 150 kHz, 1000 Hz to 100 kHz, 1000 to 50 kHz, or 1000 Hz to 25 kHz, or 1000 Hz to 10 kHz, or 1000 Hz to 5000 Hz, or 1000 Hz to 3000 Hz, or 1000 Hz to 1500 Hz, or 1000 Hz to 1000 Hz. In other embodiments, the frequencies range from about 200 Hz to 10 kHz, 200 Hz to 9000 Hz, 200 Hz to 8000 Hz, 200 Hz to 7000 Hz, 200 to 6000 Hz, 200 Hz to 5000 Hz, 200 Hz to 4000 Hz, 200 Hz to 3000 Hz, or 200 Hz to 2000 Hz, or 200 Hz to 1000 Hz. In other embodiments, the frequencies range from about 1000 Hz to 10 kHz, 1000 Hz to 9000 Hz, 1000 Hz to 8000 Hz, 1000 Hz to 7000 Hz, 1000 to 6000 Hz, 1000 Hz to 5000 Hz, 1000 Hz to 4000 Hz, 1000 Hz to 3000 Hz, or 1000 Hz to 2000 Hz. In embodiments, electrical signals at such frequencies can downregulate nerve activity.

In some embodiments, a user selects a frequency of 300 Hz or less. In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can stimulate nerve activity.

Optionally, a user may select a pulse width for each charge and recharge phase. The pulse width chosen for a particular frequency will depend on whether one or more pulse delays are included within the period. In embodiments, pulse delay selections include but are not limited to at least 5 microseconds, 10 microseconds, 20 microseconds, or 30 microseconds.

In embodiments, a user may select the number of periods in a microsecond cycle. In embodiments, the number of periods is at least 2 periods. In embodiments, the number periods in a microsecond cycle can range for 2 to 20, 2 to 15, 2 to 10, or 2 to 5 periods in a microsecond cycle. In embodiments, a user may select the number of stimulation periods in a stimulation cycle. In embodiments, the number of stimulation periods is at least 2 stimulation periods. In embodiments, the number periods in a stimulation cycle can range for 2 to 20, 2 to 15, 2 to 10, or 2 to 5 periods in a stimulation cycle. A user may also select a first and/or second amplitude. In embodiments, the first selected amplitude is applied to first pattern of electrical signal treatment. In embodiments, a second selected amplitude is applied to a second pattern of electrical signal treatment, where the first and second amplitudes are different from one another. The selections of amplitudes include about 0.01 to 20 mAmps, 0.01 to 15 mAmps, 0.01 to 10 mAmps, 0.01 to 8 mAmps, or 0.01 to 5 mAmps. In embodiments, the first and/or second amplitude is constant during the time period of the electrical signal treatment. In embodiments, the amplitude is at least 1 volt. In other embodiments, the amplitude ranges from about 0.01 to 20 volts, 0.01 to 15 volts, 0.01 to 10 volts, 0.01 to 8 volts, or 0.01 to 5 volts. In embodiments, a single amplitude or voltage is selected.

In yet other embodiments, a user can select a ramp up and/or a ramp down time for amplitude and/or pulse width and/or frequency. During the ramp up and ramp down time the amplitude or pulse width or frequency is changing. In embodiments, the amplitudes for ramp up include about 0.01 to 20 mAmps, 0.01 to 15 mAmps, 0.01 to 10 mAmps, 0.01 to 8 mAmps, or 0.01 to 5 mAmps. In embodiments, the amplitude for a ramp up is at least 0.01 volt. In other embodiments, the amplitude ranges from about 0.01 to 20 volts, 0.01 to 15 volts, 0.01 to 10 volts, 0.01 to 8 volts, or 0.01 to 5 volts. In embodiments, the time or ramp up and/or ramp down is about 200 microseconds to 25 milliseconds for high frequency signals. In embodiments, the time or ramp up and/or ramp down is about 10 seconds to 15 minutes for low frequency stimulation signals.

In embodiments, a user can select a microsecond inactive phase time for high frequency signals. In embodiments, the microsecond inactive phase is at least about 80 microseconds. In embodiments, the microsecond inactive phase is at least 80 microseconds up to 10,000 microseconds, 200 microseconds up to 10,000 microseconds, or 400 microseconds up to 10,000 microseconds.

In embodiments, a user can select a millisecond active phase. In embodiments, the millisecond active phase is at least 0.16 millisecond. In embodiments, the millisecond active phase is 0.16 millisecond to 1,100 milliseconds, 0.16 millisecond to 900 milliseconds, 0.16 millisecond to 800 milliseconds, 0.16 millisecond to 700 milliseconds, 0.16 millisecond to 600 milliseconds, 0.16 millisecond to 500 milliseconds, 0.16 to 400 milliseconds, 0.16 to 300 milliseconds, 0.16 to 200 milliseconds, 0.16 to 100 milliseconds, 0.16 to 50 milliseconds, 0.16 to 40 milliseconds, 0.16 to 30 milliseconds, 0.16 to 20 milliseconds, 0.16 to 10 milliseconds, or 0.16 to 5 milliseconds. In embodiments, the millisecond active phase is at least 1 millisecond. In other embodiments, the millisecond active phase is 1 to 1,100 milliseconds, 1 millisecond to 900 milliseconds, 1 millisecond to 800 milliseconds, 1 millisecond to 700 milliseconds, 1 millisecond to 600 milliseconds, 1 millisecond to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, 1 to 10 milliseconds, or 1 to 5 milliseconds.

In embodiments, a user can select the time of a millisecond inactive phase for high frequency signals. In embodiments, the millisecond inactive phase is at least 0.08 milliseconds. In embodiments, the millisecond inactive phase is 0.08 millisecond to 11,000 milliseconds, 0.08 millisecond to 9000 milliseconds, 0.08 millisecond to 8000 milliseconds, 0.08 millisecond to 7000 milliseconds, 0.08 millisecond to 6000 milliseconds, 0.08 millisecond to 5000 milliseconds, 0.08 to 4000 milliseconds, 0.08 to 3000 milliseconds, 0.08 to 2000 milliseconds, 0.08 to 1000 milliseconds, 0.08 to 500 milliseconds, 0.08 to 400 milliseconds, 0.08 to 300 milliseconds, 0.08 to 200 milliseconds, 0.08 to 100 milliseconds, 0.08 to 50 milliseconds, 0.08 to 40 milliseconds, 0.08 to 30 milliseconds, 0.08 to 20 milliseconds, or 0.08 to 10 milliseconds. In embodiments, the millisecond inactive phase is 1 millisecond to 11,000 milliseconds, 1 millisecond to 9000 milliseconds, 1 millisecond to 8000 milliseconds, 1 millisecond to 7000 milliseconds, 1 millisecond to 6000 milliseconds, 1 millisecond to 5000 milliseconds, 1 to 4000 milliseconds, 1 to 3000 milliseconds, 1 to 2000 milliseconds, 1 to 1000 milliseconds, 1 to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, or 1 to 10 milliseconds.

In embodiments, a user can select time of a stimulation active phase for a low frequency stimulation signal. In embodiments, the stimulation active phase is at least about 10 seconds. In embodiments, the stimulation active phase is about 10 seconds to about 30 minutes, about 10 seconds to about 25 minutes, about 10 seconds to about 20 minutes, about 10 seconds to about 15 minutes, about 10 seconds to about 10 minutes, about 10 seconds to about 5 minutes, about 10 seconds to about 1 minute, about 10 seconds to about 30 seconds, or about 20 seconds to about 30 minutes, about 30 seconds to about 30 minutes, about 40 seconds to about 30 minutes, about 50 seconds to about 30 minutes, about 1 minute to about 30 minutes, about 5 minutes to about 30 minutes, about 10 minutes to about 30 minutes, about 15 minutes to about 30 minutes, about 20 minutes to about 30 minutes, about 25 minutes to about 30 minutes.

In embodiments, a user can select the time of a stimulation inactive phase for a low frequency stimulation signal. In embodiments, the stimulation inactive phase is about 0.01 seconds to about 100 seconds. In embodiments, a stimulation inactive phase is about 100 seconds or less, about 50 seconds or less, or about 10 second or less, or about 5 seconds or less, or about 1 seconds or less, or about 0.1 seconds or less, or about 0.01 seconds or less. In embodiments, the stimulation inactive phase is at least 0.01 seconds up to 100 seconds, 0.01 seconds up to 50 seconds, 0.01 seconds up to 10 seconds, 0.01 seconds up to 5 seconds, or 0.01 seconds up to 1 second, or 0.01 seconds up to 0.5 seconds, or 0.01 seconds up to 0.2 seconds, or 0.01 seconds up to 0.1 seconds.

In embodiments, a user can select an on time for a high frequency signal. In embodiments, an on time can be selected from 30 seconds to about 30 minutes, 30 seconds to about 15 minutes, 30 seconds to about 10 minutes, 30 seconds to about 5 minutes, 30 seconds to about 2 minutes, or 30 seconds to about 1 minute.

In embodiments, a user can select an off time for a high frequency signal. In embodiments, off times are at least about 30 seconds. In other embodiments, the off time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute.

Optionally, a user may select a percentage of duty cycle for a microsecond and/or millisecond cycle. In embodiments, a user can select a duty cycle of 100% or less, 90% or less, 80% or less, 70% or less, 60% or less, 50% or less, 40% or less, 30% or less, 20% or less, or 10% or less. In embodiments, a user can select a duty cycle of 75% or less.

In embodiments, a user can select an on time for a low frequency stimulation signal. In embodiments, the on time is at least about 30 seconds. In other embodiments, the on time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include on times of varying amounts. For example, a therapy cycle can include 1 minutes of on time, 1 minute of off time, 2 minutes of on time, followed by 5 minutes of off time.

In embodiments, a user can select an off time for a low frequency stimulation signal. In embodiments, the off time is selected in order to allow at least partial recovery of the nerve. In embodiments, the off time may be minimized due to the presence of stimulation inactive phases and/or idle phases. In embodiments, off times are at least about 30 seconds. In other embodiments, the off time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include off times of varying amounts. For example, a therapy cycle can include 1 minute of on time, 1 minute of off time, 2 minutes of on time, followed by 5 minutes of off time.

In some embodiments, a user can select parameters for multiple electrical signals that are to be applied to multiple nerves or multiple nerve branches/fibers of a subject in one or more related therapy programs. The parameters of each electrical signal is independently and separately adjustable by the user, and may be concurrently applied and/or applied in a coordinated fashion to the multiple nerves or multiple nerve branches/fibers of the subject.

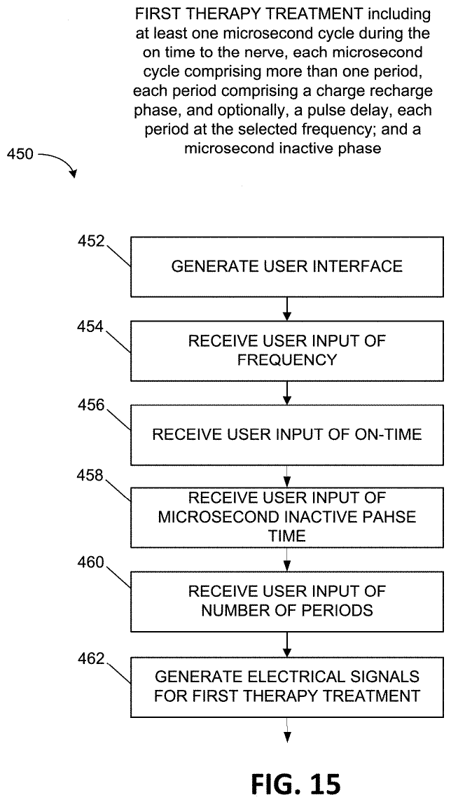

Referring to FIGS. 15-17, example methods for operating the therapy system 100 are described for different therapy programs. In the illustrated examples, three different therapy programs are described. However, the system 100 can operate to provide other therapy programs in accordance with the present disclosure.

FIG. 15 is a flowchart illustrating an example method 450 for operating the therapy system 100 for a first therapy program. At operation 452, the system 100 generates a user interface for enabling a user to input one or more parameters for the first therapy program. At operation 454, the system 100 receives a user input of a frequency parameter via the user interface. At operation 456, the system 100 receives a user input of an on-time parameter via the user interface. At operation 458, the system 100 receives a user input of a microsecond inactive time parameter via the user interface. At operation 460, the system 100 receives a user input of a number of periods via the user interface. In some embodiments, the system 100 receives additional parameters including, but not limited to, amplitude, a pulse width or pulse delay time, and a ramp up/down time. In other embodiments, the system 100 receives only some of the parameters described above. At operation 462, the system 100 generates electrical signals for the first therapy treatment based on the inputted parameters. The first therapy treatment can include a high frequency signal comprising at least one microsecond cycle during the on time to the nerve, each microsecond cycle comprising more than one period, each period comprising a charge recharge phase, and optionally, a pulse delay, each period at the selected frequency; and a microsecond inactive phase. It should be noted that the first therapy treatment can also include a low frequency stimulation signal (not shown in FIG. 15) comprising at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. The first therapy treatment can alternatively include both a high frequency signal and a low frequency stimulation signal (not shown in FIG. 15) and the corresponding parameters thereof, wherein the high frequency signal for blocking nerve activity is applied to a first nerve or nerve branch/fiber or organ, and the low frequency stimulation signal for stimulating/upregulating nerve activity is applied to a second nerve or nerve branch/fiber or an organ.

The method 450 illustrates one example operation of the system 100 for the first therapy program. In some embodiments, the method 450 includes only some of the operations described above. In other embodiments, the method 450 includes additional operations along with all or some of the operations described above.

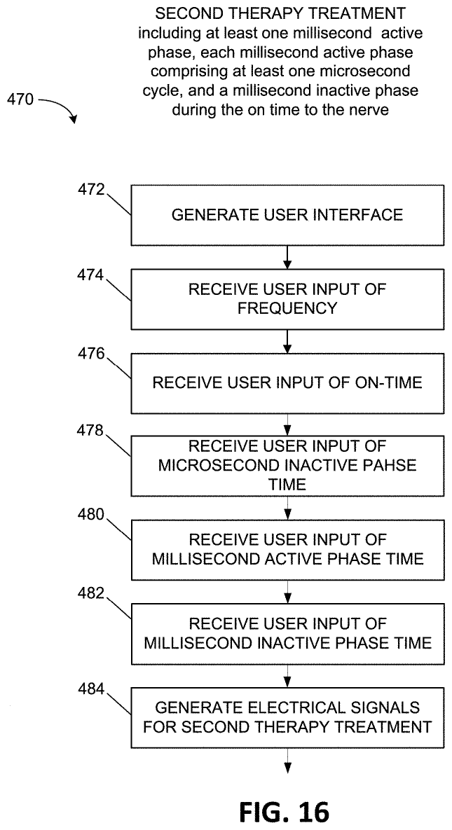

FIG. 16 is a flowchart illustrating an example method 470 for operating the therapy system 100 for a second therapy program. At operation 472, the system 100 generates a user interface for enabling a user to input one or more parameters for the second therapy program. At operation 474, the system 100 receives a user input of a frequency parameter via the user interface. At operation 476, the system 100 receives a user input of an on-time parameter via the user interface. At operation 478, the system 100 receives a user input of a microsecond inactive time parameter via the user interface. At operation 480, the system 100 receives a user input of a millisecond active phase time via the user interface. At operation 482, the system 100 receives a user input of a millisecond inactive phase time via the user interface. In some embodiments, the system 100 receives additional parameters. In other embodiments, the system 100 receives only some of the parameters described above. At operation 462, the system 100 generates electrical signals for the second therapy treatment based on the inputted parameters. The second therapy treatment can include a high frequency signal comprising at least one millisecond active phase, each millisecond active phase comprising at least one microsecond cycle, and a millisecond inactive phase during the on time to the nerve. It should be noted that the first therapy treatment can also include a low frequency stimulation signal (not shown in FIG. 16) comprising at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. The first therapy treatment can alternatively include both a high frequency signal and a low frequency stimulation signal and the corresponding parameters thereof (not shown in FIG. 16), wherein the high frequency signal for blocking nerve activity is applied to a first nerve or nerve branch/fiber or an organ, and the low frequency stimulation signal for stimulating/upregulating nerve activity is applied to a second nerve or nerve branch/fiber or an organ.

The method 470 illustrates one example operation of the system 100 for the second therapy program. In some embodiments, the method 470 includes only some of the operations described above. In other embodiments, the method 470 includes additional operations along with all or some of the operations described above.

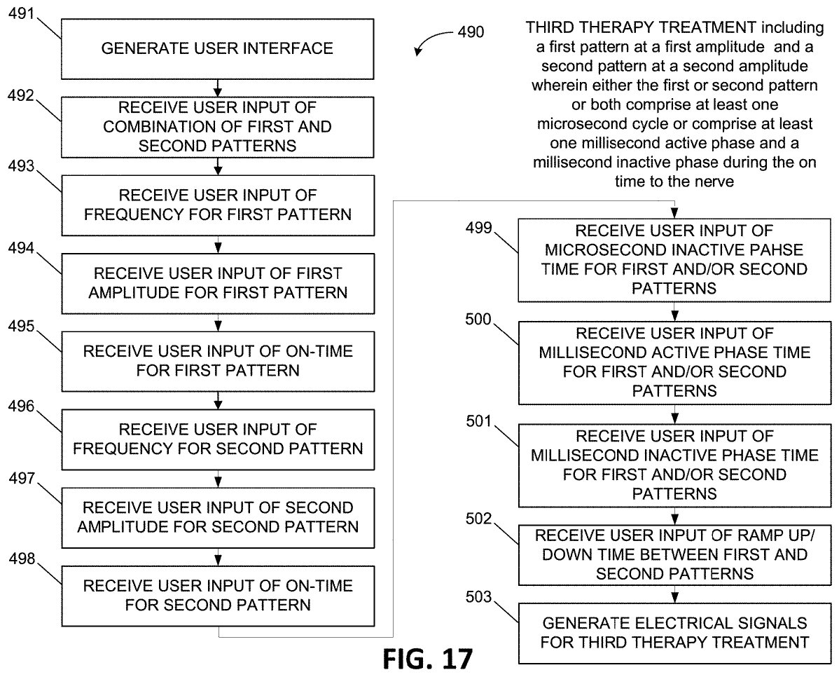

FIG. 17 is a flowchart illustrating an example method 490 for operating the therapy system 100 for a third therapy program. At operation 491, the system 100 generates a user interface for enabling a user to input one or more parameters for the third therapy program. At operation 492, the system 100 receives a user selection of a combination of first and second patterns via the user interface. At operation 493, the system 100 receives a user input of a first frequency parameter for the first pattern via the user interface. At operation 494, the system 100 receives a user input of a first amplitude for the first pattern via the user interface. At operation 495, the system 100 receives a user input of an on-time parameter for the first pattern via the user interface. At operation 496, the system 100 receives a user input of a second frequency parameter for the second pattern via the user interface. At operation 497, the system 100 receives a user input of a second amplitude for the second pattern via the user interface. At operation 498, the system 100 receives a user input of an on-time parameter for the second pattern via the user interface. At operation 499, the system 100 receives a user input of a microsecond inactive phase time parameter for the first and/or second patterns via the user interface. At operation 500, the system 100 receives a user input of a millisecond active phase time for the first and/or second patterns via the user interface. At operation 501, the system 100 receives a user input of a millisecond inactive phase time for the first and/or second patterns via the user interface. At operation 502, the system 100 receives a user input of a ramp up and/or down time parameters via the user interface. In some embodiments, the system 100 receives additional parameters. In other embodiments, the system 100 receives only some of the parameters described above. At operation 503, the system 100 generates electrical signals for the third therapy treatment based on the inputted parameters. The third therapy treatment can include a first pattern at a first amplitude and a second pattern at a second amplitude, wherein either the first or second pattern or both comprise at least one microsecond cycle or comprise at least one millisecond active phase and a millisecond inactive phase during the on time to the nerve. The methods and systems for operating the therapy system 100 for the third therapy program apply to a high frequency signal, a low frequency stimulation signal, or the combination thereof.

The method 490 illustrates one example operation of the system 100 for the third therapy program. In some embodiments, the method 490 includes only some of the operations described above. In other embodiments, the method 470 includes additional operations along with all or some of the operations described above.

As described herein, a user interface provides any one or all of the above parameters. In some embodiments, the parameters may have default values. In other embodiments, each parameters can have one or more options for selection as described herein.

In embodiments, a user interface can provide at least three different therapy programs. If a first therapy program including a high frequency signal is selected, a user may select a frequency, an on time, the number of periods, a microsecond inactive time, an amplitude, and optionally, a pulse width or pulse delay time and ramp up/down time. The first program then provides an electrical signal treatment comprising multiple microsecond cycles during the on time at the selected frequency, each microsecond cycle comprising more than one period, each period comprising a charge recharge phase, and optionally, a pulse delay, each period at the selected frequency; and a microsecond inactive phase. If a first therapy program including a low frequency stimulation signal is selected, a user may select a frequency, an on time, the number of stimulation periods, a stimulation inactive time, an amplitude, and optionally, a pulse width or pulse delay time and ramp up/down time. The first program then provides an electrical signal treatment comprising at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. If a first therapy program including both a high frequency signal applied to a first nerve and a low frequency stimulation signal applied to a second nerve is selected, a user may independently select parameters respectively for the high frequency signal and the low frequency stimulation signal.

If a second therapy program is selected, a user selects the frequency, an amplitude, a microsecond inactive phase time, a millisecond active phase time, a millisecond inactive phase time, and an on time. In embodiments, the second program then provides an electrical signal treatment that comprises at least one millisecond active phase during an on time, each millisecond active phase comprising at least microsecond cycle; and a millisecond inactive phase. If a second therapy program including a low frequency stimulation signal is selected, a user may select a frequency, an on time, the number of stimulation periods, a stimulation inactive time, an amplitude, and optionally, a pulse width or pulse delay time and ramp up/down time. The second program then provides an electrical signal treatment comprising at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. If a second therapy program including both a high frequency signal applied to a first nerve and a low frequency stimulation signal applied to a second nerve is selected, a user may independently select parameters respectively for the high frequency signal and the low frequency stimulation signal.

If a third therapy program is selected, a user selects a first pattern comprising a frequency, a first amplitude, and an on time; and a second pattern comprising a second frequency, a second amplitude; and a second on time. A user then further selects for either the first or second pattern or both, a microsecond inactive phase time, a millisecond active phase time, and a millisecond inactive phase time. Optionally a user selects a ramp up and/or ramp down time between the first and second patterns. In embodiments, the third program provides an electrical signal treatment that comprises a first pattern of electrical signal at a first amplitude and a second pattern at a second amplitude. The third therapy can optionally include a high frequency signal alone, a low frequency stimulation signal alone, or the combination thereof. In situations where the third therapy program include both a high frequency signal and a low frequency stimulation signal, a user may independently select parameters respectively for the high frequency signal and the low frequency stimulation signal.

In another aspect of the disclosure, a computer implemented method and a computer readable medium are provided. In embodiments, the computer readable medium comprises executable instructions for implementing an electrical signal therapy for downregulating and/or upregulating activity on a nerve in a subject comprising providing at least one frequency for selection, providing at least one on time for selection, providing at least one microsecond inactive phase time for selection, providing for a number of periods, and once selections are made, providing instructions for applying an electrical signal treatment comprising at least one microsecond cycle during the on time to the nerve, each microsecond cycle comprising more than one period, each period comprising a charge recharge phase, and optionally, a pulse delay, each period at the selected frequency; and a microsecond inactive phase. In other embodiments, the computer readable medium comprises executable instructions for implementing a low frequency stimulation signal therapy for stimulating/upregulating activity on a nerve in a subject comprising providing at least one stimulation cycle, providing at least one on time selection, providing at least one stimulation inactive phase time for selection, providing for a number of periods, and once selections are made, providing instructions for applying a low frequency stimulation signal treatment comprising at least one stimulation period, each of the at least one stimulation period comprising a pulse and optionally a stimulation inactive phase, wherein the pulse comprises a cathodic and/or anodic phase and optionally a pulse delay, the pulse having a pulse width. In other embodiments, a computer readable medium comprises executable instructions for implementing an electrical signal therapy for downregulating and/or upregulating activity on a nerve in a subject comprising providing at least one frequency for selection, providing at least one on time for selection, providing at least one microsecond inactive phase time for selection, providing at least one millisecond active phase time for selection, providing at least one millisecond inactive phase time for selection; and once selections are made, providing instructions for applying an electrical signal treatment comprising at least one millisecond active phase, each millisecond active phase comprising at least one microsecond cycle, and a millisecond inactive phase during the on time to the nerve. In other embodiments, a computer readable medium comprises executable instructions for implementing a low frequency stimulation signal therapy for upregulating/stimulating activity on a nerve in a subject comprising providing at least one frequency for selection, providing at least one on time for selection, providing at least one stimulation inactive phase time for selection, providing at least one stimulation active phase time for selection, providing at least one idle phase time for selection; and once selections are made, providing instructions for applying a low frequency stimulation signal treatment comprising at least one stimulation active phase, each stimulation active phase comprising at least one stimulation cycle, and an idle phase during the on time to the nerve.

In yet other embodiments, a computer readable medium comprises executable instructions for implementing an electrical signal therapy for downregulating and/or upregulating activity on a nerve comprising providing a first pattern of electrical signal comprising providing at least one frequency for selection, providing a first amplitude for selection, and providing a first on time; providing a second pattern for selection comprising providing at least one frequency, providing a second amplitude, and providing a second on time. Further embodiments, comprise providing for microsecond inactive phase time for selection in the first or second pattern or both, providing at least one millisecond active phase time for selection in the first or second pattern or both, providing at least one millisecond inactive phase time for selection in the first or second pattern or both; and once selections are made, providing instructions for applying an electrical signal treatment comprising a first pattern at a first amplitude and a second pattern at a second amplitude wherein either the first or second pattern or both comprise at least one microsecond cycle or comprise at least one millisecond active phase and a millisecond inactive phase during the on time to the nerve. Embodiments further comprise providing for a ramp up and ramp down time, and once selections are made providing instructions to apply a ramp up or ramp down time between the first and second pattern.

In embodiments, a computer implemented method comprises applying a high frequency signal to a nerve at a selected frequency, a selected on time, a selected number of periods, and a selected microsecond inactive phase time, wherein the electrical signal comprises at least one microsecond cycle during an on time, each microsecond cycle comprising more than one period, each period comprising a charge recharge phase, and optionally, a pulse delay, each period having a frequency of at least about 200 Hz; and a microsecond inactive phase. In embodiments, the method comprises selecting a frequency, selecting an on time, selecting the number of periods, and selecting a microsecond inactive phase time. In other embodiments, a computer implemented method comprises applying a low frequency stimulation signal to a nerve at a selected frequency, a selected on time, a selected number of periods, and a selected stimulation inactive phase time, wherein the low frequency stimulation signal comprises at least one stimulation cycle during an on time, each stimulation cycle comprising more than one stimulation period, each stimulation period comprising a pulse, and optionally, a pulse delay, each stimulation period having a frequency of at most about 199 Hz; and a stimulation inactive phase. In embodiments, the method comprises selecting a frequency, selecting an on time, selecting the number of stimulation periods, and selecting a stimulation inactive phase time for the low frequency stimulation signal.

In embodiments, a computer implemented method comprises applying an electrical signal to a nerve at a selected frequency, a selected on time, a selected microsecond inactive phase time, a selected millisecond active phase time, and a selected millisecond inactive phase time, wherein the electrical signal comprises at least one millisecond active phase, each millisecond active phase comprising at least one microsecond cycle, and a millisecond inactive phase during the on time to the nerve. In other embodiments, a computer implemented method comprises applying a low frequency stimulation signal to a nerve at a selected frequency, a selected on time, a selected stimulation inactive phase time, a selected idle phase time, and a selected stimulation inactive phase time, wherein the low frequency stimulation signal comprises at least one stimulation active phase, each stimulation active phase comprising at least one stimulation cycle, and an idle phase during the on time to the nerve.

In yet other embodiments, a computer implemented method comprises applying an electrical signal therapy for downregulating activity on a nerve comprising applying a first pattern of electrical signal comprising a selected frequency, a selected on time, a selected first amplitude, applying a second pattern of electrical signal comprising a selected second frequency, a selected second amplitude, and a second selected on time; further providing for a selected microsecond inactive phase time in the first or second pattern or both, providing a selected millisecond active phase time in the first or second pattern or both, providing a selected millisecond inactive phase time in the first or second pattern or both; and applying an electrical signal treatment comprising a first pattern at a first amplitude and a second pattern at a second amplitude, wherein either the first or second pattern or both comprise at least one microsecond cycle or comprise at least one millisecond active phase and a millisecond inactive phase during the on time to the nerve. Embodiments further comprises applying a selected ramp up and/ramp down time between the first and second pattern.

In some embodiments, a computer implemented method comprises applying a first electrical signal to a first nerve and applying a second electrical signal to a second nerve, wherein the first electrical signal comprises at least one microsecond cycle and optionally a microsecond inactive phase, wherein each of the at least one microsecond cycle comprises at least one period, each of the at least one period comprising a pulse comprising a charge recharge phase, the pulse having a pulse width, and wherein the second electrical signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width, and wherein the first electrical signal downregulates nerve activity and has a frequency from about 200 Hz to about 100 kHz, and wherein the second electrical signal upregulates nerve activity and has a frequency from about 0.01 Hz to 199 Hz.

5. Therapy Schedule

In embodiments, to initiate the therapy regimen, the clinician downloads therapy parameters and/or one or more therapy programs, and a therapy schedule from an external computer, smartphone or tablet 107 to the external charger 101. In general, the therapy parameters indicate configuration values for the neuroregulator 101. For example, in the case of vagal nerve therapy for obesity, the therapy parameters may define the pulse amplitude with a fixed but selectable voltage or current, frequency, microsecond inactive phase time, millisecond active phase time, millisecond inactive phase time, stimulation inactive phase, idle, pulse width, pulse delay, ramp up, ramp down, on time, off time, start time, end time, waveform shape, and pattern of electrical pulses in a cycle for the electrical signals emitted by the implanted neuroregulator 104.

In general, the therapy schedule indicates a therapy cycle start time, the number of therapy cycles, timing of therapy cycles and duration of the delivery of therapy cycles for at least one day of the week. A therapy cycle refers to a discrete period of time (e.g. on the order of minutes) that contains one or more on times and off times. The pattern of on and off times can be repetitive, non-fixed or randomized throughout a therapy schedule. Preferably, the clinician programs a therapy schedule start time and duration for each day of a predetermined period, such as a week, month, time patient is on vacation, or time to next follow-up visit. In an embodiment, multiple therapy cycles can be scheduled within a single day. Therapy can also be withheld for one or more days at the determination of the clinician.

During a therapy schedule the neuroregulator 104 completes one or more therapy cycles. Typically, each therapy schedule includes multiple therapy cycles. The clinician has the ability to program the duration of each therapy cycle (i.e., via the clinician computer, smartphone or tablet 107).

When configured in the “on” state, the neuroregulator applies therapy (i.e., emits an electrical signal) as has been described herein. The neuroregulator 104 is then cycled to an “off” state, during which no signal is emitted by the neuroregulator 104, at intermittent periods (on the order of minutes). Such a therapy cycle may mitigate the chances of accommodation by the patient's body. A long off state also has the advantage of saving energy.

The therapy schedule indicates the times during the day when one or multiple therapy cycles are scheduled to be applied to a patient. In one embodiment, as an illustrative example, one or multiple therapy cycles can be scheduled between 8 AM and 9 AM. In certain embodiments, the therapy parameters indicates details of the pulse amplitude with a fixed but selectable voltage or current, frequency, pulse width, pulse delays, microsecond inactive phase time, millisecond active phase time, millisecond inactive phase time, stimulation active phase time, stimulation inactive phase time, stimulation second cycle time, idle phase time, ramp up, ramp down, on time, off time, waveform shape and pattern of active/inactive phases in a cycle. As an illustrative example, a therapy cycle may define an on period wherein one or more sets of pulses are delivered to the nerve for two minutes, followed by an off period of one minute where no pulses are delivered. A second on period of two minutes may follow the initial off period, followed by an off period of five minutes, wherein the cycle repeats itself. The therapy schedule may then continue for a period of six to twenty four hours as determined by the physician.

In embodiments, the therapy schedule can be executed to apply multiple electrical signals to multiple nerves or nerve branches/fibers in a subject, allowing the neuroregulator to independently deliver and control each of the electrical signals applied to the corresponding nerve or nerve branch/fiber or organ.

B. Methods

1. Methods for Downregulating and/or Upregulating Nerve Activity

In some aspects, the present disclosure relates to the systems and methods of the disclosure are useful to downregulate and/or upregulate activity on a nerve of a subject including but not limited to the vagus nerve, renal nerve, renal artery, sympathetic nerves, glossopharyngeal nerve, celiac nerve, and combinations thereof. The systems and methods are useful in treating gastrointestinal disorders, obesity and eating disorders, pancreatitis and other inflammatory conditions, ulcerative colitis, Crohn's disease, diabetes, prediabetes, hypertension, and congestive heart failure.

In embodiments, a method of treating gastrointestinal disorders comprises applying a high frequency electrical signal to downregulate nerve activity to a nerve of a subject by applying the electrical signal to the nerve during an on time, wherein the electrical signal comprises more than one microsecond cycle comprising more than one period, each period comprising a charge recharge phase, and optionally, a pulse delay, each period having a frequency of at least about 200 Hz; and a microsecond inactive phase. In embodiments, parameters of the electrical signal downregulate (or block) activity of the nerve. In other embodiments, parameters of the electrical signal upregulate (or stimulate) activity of the nerve. In embodiments, the nerve is selected from the group consisting of the vagus nerve, the renal nerve, the renal artery, splanchnic nerve, celiac plexus, and combinations thereof. A nerve generally comprises one or more nerve branches/fibers.

In other embodiments, the method of treating gastrointestinal disorders comprises applying an electrical signal to a nerve of a subject, wherein the electrical signal comprises more than one microsecond cycle to form a millisecond active phase, and applying more than one millisecond active phase during the on time, wherein each millisecond active phase is separated by a millisecond inactive phase during the on time. In embodiments, the millisecond inactive phase is longer than the millisecond active phase. In embodiments, the millisecond inactive phase can vary in time between each millisecond active phase. In embodiments, parameters of the electrical signal downregulate activity of the neve. In embodiments, the nerve is selected from the group consisting of the vagus nerve, the renal nerve, the renal artery, splanchnic nerve, celiac plexus, and combinations thereof.

In yet other embodiments, a method of treating gastrointestinal disorders comprises applying a high frequency electrical signal to a nerve of a subject by applying the electrical signal to the nerve during an on time, wherein the electrical signal comprises a first pattern comprising at least one microsecond cycle; and a second pattern comprising more than one millisecond active phase, wherein each millisecond active phase comprises more than one microsecond cycle, and each millisecond active phase is separated by a millisecond inactive phase. In embodiments, the first and second patterns have a different amplitude. In embodiments, the microsecond cycle comprises at least one period comprising a charge recharge phase, and optionally, a pulse delay, each period having a frequency of at least about 200 Hz; and a microsecond inactive phase. In embodiments, the first pattern has an amplitude greater than the second pattern. In embodiments, the first pattern has an on time and the second pattern has on times that differ from one another. In embodiments, the nerve is selected from the group consisting of the vagus nerve, the renal nerve, the renal artery, splanchnic nerve, celiac plexus, and combinations thereof.

In embodiments, methods for treating gastrointestinal conditions are performed where the nerve is selected from the vagus nerve and its individual branches and/or splanchnic nerve, and/or celiac complex. In embodiments, at least one electrode is placed on or near the vagus nerve. In embodiments, gastrointestinal disease includes obesity, overweight, pancreatitis, dysmotility, bulimia, gastrointestinal disease with an inflammatory basis such as ulcerative colitis and Crohn's disease, low vagal tone, gastroparesis, reflux disease, peptic ulcers, or combinations thereof. In embodiments, the electrical signal therapy can be combined with administration of therapeutic agents that affect energy regulation. In embodiments, the methods include the electrical signal parameters, systems, computer readable media, and computer implemented methods as described herein.

In embodiments, a method of treating disorders of blood glucose regulation comprises applying an electrical signal having parameters that downregulate nerve activity to a nerve of a subject by applying the electrical signal to the nerve during an on time, wherein the electrical signal comprises more than one microsecond cycle comprising at least one period comprising a charge recharge phase and optionally, a pulse delay, each period having a frequency of at least about 200 Hz; and a microsecond inactive phase.

In other embodiments, the method of treating disorders of blood glucose regulation comprises applying an electrical signal to a nerve of a subject, wherein the electrical signal comprises more than one microsecond cycle to form a millisecond active phase, and applying more than one millisecond active phase during the on time, wherein each millisecond active phase is separated by a millisecond inactive phase during the on time. In embodiments, the millisecond inactive phase is longer than the millisecond active phase. In embodiments, the millisecond inactive phase can vary in time between each millisecond active phase.

In yet other embodiments, a method of treating disorders of blood glucose regulation comprises applying an electrical signal having a frequency to downregulate nerve activity to a nerve of a subject by applying the electrical signal to the nerve during an on time, wherein the electrical signal comprises a first pattern comprising at least one microsecond cycle; and a second pattern comprising more than one millisecond active phase, wherein each millisecond active phase comprises more than one microsecond cycle, and each millisecond active phase is separated by a millisecond inactive phase, wherein the first and second patterns have a different amplitude. In embodiments, the microsecond cycle comprises at least one period comprising a charge recharge phase, and optionally, a pulse delay, wherein each period has a frequency of at least 200 Hz; and a microsecond inactive phase. In embodiments, the first pattern has an amplitude greater than the second pattern.

In embodiments, the methods for treating disorders of glucose regulation, the nerve is selected from the group consisting of the vagus nerve, sympathetic nerves, splanchnic nerve, celiac plexus, and combinations thereof. In embodiments, at least one electrode is placed on or near the vagus nerve.

In embodiments, disorders of glucose regulation include diabetes, prediabetes, metabolic syndrome or combinations thereof. In embodiments, the methods of treating disorders of glucose regulation include also treating the disorders in combinations with drugs used to treat, diabetes, or prediabetes such as insulin and analogs thereof, GLP1 agonists, sulfonylureas, and the like. In embodiments, the methods include the electrical signal parameters, systems, computer readable media, and computer implemented methods as described herein.

In embodiments, a method of treating diabetes or a condition associated with impaired glucose regulation in a subject comprises applying an electrical signal having parameters that downregulate nerve activity to a nerve in a subject by applying the electrical signal to the nerve during an on time, wherein the electrical signal comprises at more than one microsecond cycle comprising more than one period comprising a charge recharge phase, and optionally, a pulse delay, each period has a frequency of at least about 200 Hz; and a microsecond inactive phase. In other embodiments, the method of treating diabetes or a condition associated with impaired glucose regulation comprises applying an electrical signal to a nerve of a subject, wherein the electrical signal comprises more than one microsecond cycle to form a millisecond active phase, and applying more than one millisecond active phase during the on time, wherein each millisecond active phase is separated by a millisecond inactive phase during the on time. In embodiments, the millisecond inactive phase is longer than the millisecond active phase. In embodiments, the millisecond inactive phase can vary in time between each millisecond active phase.

In yet other embodiments, a method of treating diabetes or a condition associated with impaired glucose regulation in a subject comprises applying an electrical signal having a frequency to downregulate nerve activity to a nerve of a subject by applying the electrical signal to the nerve during an on time, wherein the electrical signal comprises a first pattern comprising at least one microsecond cycle; and a second pattern comprising more than one millisecond active phase, wherein each millisecond active phase comprises more than one microsecond cycle, and each millisecond active phase is separated by a millisecond inactive phase. In embodiments, the first and second patterns have a different amplitude. In embodiments, the microsecond cycle comprises at least one period comprising a charge recharge phase, and optionally, a pulse delay, wherein each period has a frequency of at least about 200 Hz; and a microsecond inactive phase. In embodiments, the first pattern has an amplitude greater than the second pattern. In embodiments, the method further comprises applying a ramp up and/or ramp down time between the first and second patterns. In embodiments, the methods of treating diabetes or a condition associated with impaired glucose regulation, wherein the nerve is selected from the group consisting of the vagus nerve, renal nerve, renal artery, sympathetic nerves, baroreceptors, glossopharyngeal nerve, and combinations thereof. In embodiments, at least one electrode is placed on or near the vagus nerve. In other embodiments, at least one electrode is placed on or near renal nerve, renal artery, sympathetic nerves, baroreceptors, glossopharyngeal nerve, and/or on the vagus nerve.

In embodiments, the methods of treating disorders described above include the parameters as described herein with regard to methods of downregulating and/or upregulating nerve activity of nerve includes those of frequency, on times, amplitudes, ramp up and ramp down times.

In embodiments, the electrical signal has a frequency in each period of a microsecond cycle of at least 200 Hz, at least 250 Hz, at least 300 Hz, at least 400 Hz, at least 500 Hz, at least 1000 Hz, at least 2000 Hz, at least 3000 Hz, at least 4000 Hz, or at least 5000 Hz, or at least 10,000 Hz, or at least 20,000 Hz, or at least 30,000 Hz, or at least 40,000 Hz, or at least 50,000 Hz, or at least 60,000 Hz, or at least 70,000 Hz, or at least 80,000 Hz, or at least 90,000 Hz, or at least 100,000 Hz, or at least 150 kHz, or at least 200 kHz, or at least 250 kHz or more. In other embodiments, the frequencies range from about 200 Hz to 250 kHz, 200 Hz to 200 kHz, 200 Hz to 150 kHz, 200 Hz to 100 kHz, 200 to 50 kHz, 200 Hz to 25 k Hz, 200 Hz to 10 kHz, or 200 Hz to 5000 Hz, or 200 Hz to 3000 Hz. or 200 Hz to 1500 Hz, or 200 to 1000 Hz. In other embodiments, the frequencies range from about 1000 Hz to 250 kHz, 1000 Hz to 200 kHz, 1000 Hz to 150 kHz, 1000 Hz to 100 kHz, 1000 to 50 kHz, or 1000 Hz to 25 kHz, or 1000 Hz to 10 kHz, or 1000 Hz to 5000 Hz, or 1000 Hz to 3000 Hz, or 1000 Hz to 1500 Hz, or 1000 Hz to 1000 Hz. In other embodiments, the frequencies range from about 200 Hz to 10 kHz, 200 Hz to 9000 Hz, 200 Hz to 8000 Hz, 200 Hz to 7000 Hz, 200 to 6000 Hz, 200 Hz to 5000 Hz, 200 Hz to 4000 Hz, 200 Hz to 3000 Hz, or 200 Hz to 2000 Hz, or 200 Hz to 1000 Hz. In other embodiments, the frequencies range from about 1000 Hz to 10 kHz, 1000 Hz to 9000 Hz, 1000 Hz to 8000 Hz, 1000 Hz to 7000 Hz, 1000 to 6000 Hz, 1000 Hz to 5000 Hz, 1000 Hz to 4000 Hz, 1000 Hz to 3000 Hz, or 1000 Hz to 2000 Hz. In embodiments, electrical signals at such frequencies can downregulate nerve activity.

In some embodiments, a user selects a frequency of 300 Hz or less. In embodiments, the electrical signal has a frequency of a period in a microsecond cycle. In embodiments, a period has a frequency of 300 Hz or less, 250 Hz or less, 200 Hz or less, 150 Hz or less, 100 Hz or less, 50 Hz or less, 10 Hz or less, 1 Hz or less. In embodiments, the electrical signal has a frequency of about 0.1 to 300 Hz, 0.1 to 250 Hz, 0.1 to 200 Hz, 0.1 to 150 Hz, 0.1 to 100 Hz, 0.1 to 50 Hz, 0.1 to 10 Hz, or 0.1 to 1 Hz. In embodiments, electrical signals at such frequencies can stimulate nerve activity.

In embodiments, the amplitude of the signal is at least 0.01 mAmp. In other embodiments, the amplitude ranges from about 0.01 to 20 mAmps, 0.01 to 15 mAmps, 0.01 to 10 mAmps, 0.01 to 8 mAmps, or 0.01 to 5 mAmps.

In embodiments, the amplitude is at least 1 volt. In other embodiments, the amplitude ranges from about 0.01 to 20 volts, 0.01 to 15 volts, 0.01 to 10 volts, 0.01 to 8 volts, or 0.01 to 5 volts.

In yet other embodiments, a user can select a ramp up and/or a ramp down time and amplitude. During the ramp up and ramp down time the amplitude is changing. In embodiments, the amplitudes for ramp up include about 0.01 to 20 mAmps, 0.01 to 15 mAmps, 0.01 to 10 mAmps, 0.01 to 8 mAmps, or 0.01 to 5 mAmps. In embodiments, the amplitude for a ramp up is at least 0.01 volt. In other embodiments, the amplitude ranges from about 0.01 to 20 volts, 0.01 to 15 volts, 0.01 to 10 volts, 0.01 to 8 volts, or 0.01 to 5 volts. In embodiments, the time or ramp up and/or ramp down is about 200 microseconds to 25 milliseconds.

In embodiments, the microsecond inactive phase is at least about 80 microseconds. In embodiments, the microsecond inactive phase is at least 80 microseconds up to 10,000 microseconds, 200 microseconds up to 10,000 microseconds, or 400 microseconds up to 10,000 microseconds.

In embodiments, the millisecond active phase is at least 0.16 millisecond. In embodiments, the millisecond active phase is 0.16 millisecond to 1,100 milliseconds, 0.16 millisecond to 900 milliseconds, 0.16 millisecond to 800 milliseconds, 0.16 millisecond to 700 milliseconds, 0.16 millisecond to 600 milliseconds. 0.16 millisecond to 500 milliseconds, 0.16 to 400 milliseconds, 0.16 to 300 milliseconds, 0.16 to 200 milliseconds, 0.16 to 100 milliseconds, 0.16 to 50 milliseconds, 0.16 to 40 milliseconds, 0.16 to 30 milliseconds, 0.16 to 20 milliseconds, 0.16 to 10 milliseconds, or 0.16 to 5 milliseconds. In embodiments, the millisecond active phase is at least 1 millisecond. In other embodiments, the millisecond active phase is 1 to 1,100 milliseconds, 1 millisecond to 900 milliseconds, 1 millisecond to 800 milliseconds, 1 millisecond to 700 milliseconds, 1 millisecond to 600 milliseconds, 1 millisecond to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, 1 to 10 milliseconds, or 1 to 5 milliseconds.

In embodiments, the millisecond active phase comprises at least 2 to 100 microsecond cycles, at least 2 to 90, at least 2 to 80, at least 2 to 70, at least 2 to 60, at least 2 to 50, at least 2 to 40, at least 2 to 30, at least 2 to 20, at least 2 to 10, at least 2 to 5, or at least 2 to 4 microsecond cycles.

In embodiments, the millisecond inactive phase is in a ratio to the millisecond active phase of about 10 to 1, 8 to 1, 6 to 1, 4 to 1, 2 to 1 or 1 to 2. In embodiments, the millisecond inactive phase is at least 0.08 milliseconds. In embodiments, the millisecond inactive phase is 0.08 millisecond to 11,000 milliseconds, 0.08 millisecond to 9000 milliseconds, 0.08 millisecond to 8000 milliseconds, 0.08 millisecond to 7000 milliseconds, 0.08 millisecond to 6000 milliseconds, 0.08 millisecond to 5000 milliseconds, 0.08 to 4000 milliseconds, 0.08 to 3000 milliseconds, 0.08 to 2000 milliseconds, 0.08 to 1000 milliseconds, 0.08 to 500 milliseconds, 0.08 to 400 milliseconds, 0.08 to 300 milliseconds, 0.08 to 200 milliseconds, 0.08 to 100 milliseconds, 0.08 to 50 milliseconds, 0.08 to 40 milliseconds, 0.08 to 30 milliseconds, 0.08 to 20 milliseconds, or 0.08 to 10 milliseconds. In embodiments, the millisecond inactive phase is 1 millisecond to 11,000 milliseconds, 1 millisecond to 9000 milliseconds, 1 millisecond to 8000 milliseconds, 1 millisecond to 7000 milliseconds, 1 millisecond to 6000 milliseconds, 1 millisecond to 5000 milliseconds, 1 to 4000 milliseconds, 1 to 3000 milliseconds, 1 to 2000 milliseconds, 1 to 1000 milliseconds, 1 to 500 milliseconds, 1 to 400 milliseconds, 1 to 300 milliseconds, 1 to 200 milliseconds, 1 to 100 milliseconds, 1 to 50 milliseconds, 1 to 40 milliseconds, 1 to 30 milliseconds, 1 to 20 milliseconds, or 1 to 10 milliseconds.

In embodiments, the on time is at least about 30 seconds. In other embodiments, the on time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow at least partial recovery of the nerve. In embodiments, the off time may be minimized due to the presence of microsecond inactive phases and/or millisecond inactive phases. In embodiments, off times are at least about 30 seconds. In other embodiments, the off time is about 30 seconds to 30 minutes, about 30 seconds to 25 minutes, about 30 seconds to 20 minutes, about 30 seconds to 15 minutes, about 30 seconds to 10 minutes, about 30 seconds to 5 minutes, about 30 seconds to 4 minutes, about 30 seconds to 3 minutes, about 30 seconds to 2 minutes, or about 30 seconds to one minute. In embodiments, a therapy cycle can include off times of varying amounts. For example, a therapy cycle can include 1 minutes of on time, 1 minute of off time, 2 minutes of on time, followed by 5 minutes of off time.

2. Methods for Downregulating Nerve Activity with High Frequency Low Duty Cycle Signals

In some aspects, the present disclosure relates to a method for regulating nerve activity of a subject comprising applying a low duty cycle electrical signal having parameters that downregulate nerve activity to a nerve of a subject by applying the electrical signal to the nerve during an on time, wherein the electrical signal comprises more than one microsecond cycle comprising at least one period comprising a charge recharge phase and optionally, a pulse delay, each period having a frequency of about 200 Hz to about 100 kHz; and a microsecond inactive phase. In some embodiments, the method for regulating nerve activity of a subject comprises applying a low duty cycle electrical signal to the nerve of the subject, wherein the electrical signal comprises more than one microsecond cycle to form a millisecond active phase, and applying more than one millisecond active phase during the on time, wherein each millisecond active phase is separated by a millisecond inactive phase during the on time. In embodiments, the millisecond inactive phase is longer than the millisecond active phase. In embodiments, the millisecond inactive phase can vary in time between each millisecond active phase.

In some embodiments, the low duty cycle electrical signal for downregulating the nerve activity has duty cycle about 75% or less, 70% or less, 60% or less, 50% or less, 40% or less, 30% or less, 20% or less, and 10% or less. In preferred embodiments, the duty cycle of the electrical signal is about 50% or less.

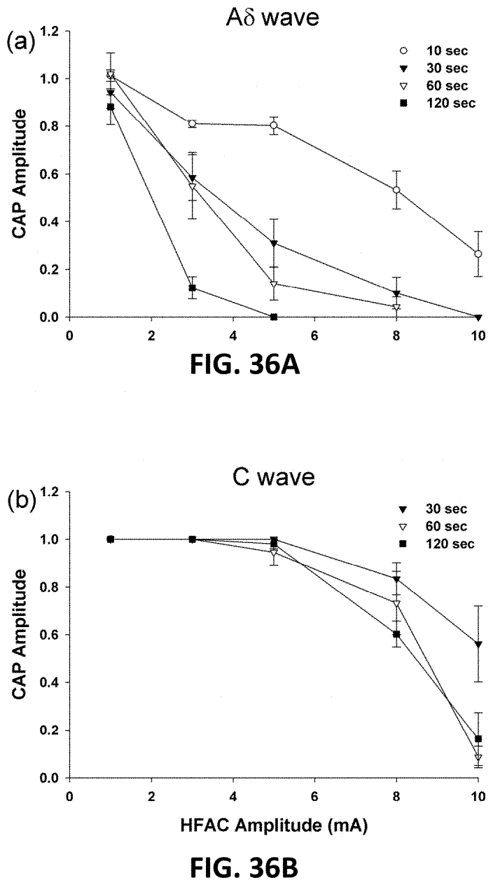

As an exemplary example shown in FIG. 37(b), the high frequency low duty cycle electrical signal comprises more than one microsecond cycle, each microsecond cycle comprises a period comprising a charge recharge phase having a pulse width of 90 microseconds for each of the charge phase and the recharge phase, and a pulse delay of 10 microseconds between the charge phase and recharge phase. The electrical signal also comprises a microsecond inactive phase of 820 microseconds. The microsecond cycle is thus 1000 microseconds, having a frequency of 1,000 Hz. The duty cycle of the electrical signal is thus 180 microsecond/1000 microsecond, or 18%.

As another exemplary example shown in FIG. 37(c), the high frequency low duty cycle electrical signal comprises more than one millisecond active phase, each millisecond active phase is 40 milliseconds and is separated by a millisecond inactive phase. Each millisecond inactive phase is of 20 millisecond. Each millisecond active phase is composed of 40 microsecond active phases, and each microsecond active phase is of 1,000 microseconds having a frequency of 1,000 Hz. Each microsecond active phase comprises a period comprising a charge recharge phase having a pulse width of 90 microseconds for each of the charge phase and the recharge phase, and a pulse delay of 10 microseconds between the charge phase and recharge phase. Each microsecond cycle also comprises a microsecond inactive phase of 820 microseconds. The duty cycle is therefore 40*180 microsecond/60 millisecond=12%.

In some embodiments, the pulse width of the high frequency low duty electrical signal is in a range from about 10 microseconds to about 500 microseconds, or from about 20 microseconds to about 450 microseconds, or from about 30 microseconds to about 400 microseconds, or from about 40 microseconds to about 350 microseconds, or from about 50 microseconds to about 300 microseconds, or from about 60 microseconds to about 250 microseconds, or from about 70 microseconds to about 200 microseconds, or from about 80 microseconds to about 150 microseconds, or from about 90 microseconds to about 100 microseconds.

In some embodiments, the microsecond inactive phases of the high frequency low duty electrical signal is in a range from about 500 microseconds to about 1,000 microseconds, or from about 550 microseconds to about 950 microseconds, or from about 600 microseconds to about 900 microseconds, or from about 650 microseconds to about 850 microseconds, or from about 700 microseconds to about 850 microseconds, or from about 750 microseconds to about 850 microseconds.

In some embodiments, the microsecond cycle of the high frequency low duty electrical signal is in a range from about 500 microseconds to about 5,000 microseconds.

In embodiments, the frequency of the high frequency low duty electrical signal is at least 200 Hz, at least 250 Hz, at least 300 Hz, at least 400 Hz, at least 500 Hz, at least 1000 Hz, at least 2000 Hz, at least 3000 Hz, at least 4000 Hz, or at least 5000 Hz, or at least 10,000 Hz, or at least 20,000 Hz, or at least 30,000 Hz, or at least 40,000 Hz, or at least 50,000 Hz, or at least 60,000 Hz, or at least 70,000 Hz, or at least 80,000 Hz, or at least 90,000 Hz, or at least 100,000 Hz, or at least 150 kHz, or at least 200 kHz, or at least 250 kHz or more. In other embodiments, the frequencies range from about 200 Hz to 250 kHz, 200 Hz to 200 kHz, 200 Hz to 150 kHz, 200 Hz to 100 kHz, 200 to 50 kHz, 200 Hz to 25 k Hz, 200 Hz to 10 kHz, or 200 Hz to 5000 Hz, or 200 Hz to 3000 Hz, or 200 Hz to 1500 Hz, or 200 to 1000 Hz. In other embodiments, the frequencies range from about 1000 Hz to 250 kHz, 1000 Hz to 200 kHz, 1000 Hz to 150 kHz, 1000 Hz to 100 kHz, 1000 to 50 kHz, or 1000 Hz to 25 kHz, or 1000 Hz to 10 kHz, or 1000 Hz to 5000 Hz, or 1000 Hz to 3000 Hz, or 1000 Hz to 1500 Hz, or 1000 Hz to 1000 Hz. In other embodiments, the frequencies range from about 200 Hz to 10 kHz, 200 Hz to 9000 Hz, 200 Hz to 8000 Hz, 200 Hz to 7000 Hz, 200 to 6000 Hz, 200 Hz to 5000 Hz, 200 Hz to 4000 Hz, 200 Hz to 3000 Hz, or 200 Hz to 2000 Hz, or 200 Hz to 1000 Hz. In other embodiments, the frequencies range from about 1000 Hz to 10 kHz, 1000 Hz to 9000 Hz, 1000 Hz to 8000 Hz, 1000 Hz to 7000 Hz, 1000 to 6000 Hz, 1000 Hz to 5000 Hz, 1000 Hz to 4000 Hz, 1000 Hz to 3000 Hz, or 1000 Hz to 2000 Hz. In embodiments, electrical signals at such frequencies can downregulate nerve activity.

In other embodiments, the frequency of the high frequency low duty electrical signal is in a range from about 200 Hz to about 2,000 Hz, or from about 400 Hz to about 1,800 Hz, or from about 600 Hz to about 1,600 Hz, or from about 800 Hz to about 1,400 Hz, or from about 900 Hz to about 1,200 Hz, or from about 1,000 Hz to about 1,100 Hz.

In some embodiments, the millisecond active phase of the high frequency low duty electrical signal is in a range from about 1 millisecond to about 500 milliseconds, or from about 5 milliseconds to about 300 milliseconds, or from about 15 milliseconds to about 100 milliseconds, or from about 20 milliseconds to about 50 milliseconds.

It is surprisingly found that the high frequency low duty electrical signal can effectively downregulate nerve activity or induce conduction block, resulting in a degree of block compared with the traditional high frequency electrical signal have duty cycle above 90% duty. The high frequency low duty electrical signal can therefore significantly reduce the energy consumption without sacrificing the performance or efficacy. Without wishing to be bound by a particular theory, it is believed that the recovery from HFAC-induced conduction block can take minutes, and this persistence of nerve blockage beyond the duration of HFAC delivery produces a “carry-over effect,” which has been observed in may nerve systems including but not limited to thinly myelinated and non-myelinated fibers. The discontinuous application of HFAC comprising low duty cycle electrical signals according to the present application may make use of the persistence of nerve blockage in the microsecond and/or millisecond inactive phases, thereby maintaining nerve blockage and lowering the power consumption.

In embodiments, the duty cycle of the present high frequency electrical signal can be decreased by order(s) of magnitude compared with traditional HFAC without significant inactive phases. The significant reduction of power consumption provides opportunities for miniaturization of neuroregulator or similar bio-electronic devices for nerve blockage.

3. Methods and Systems for Stimulating/Upregulating Nerve Activity with Low Frequency Stimulation Signals

In embodiments, a system and method for upregulating/stimulating nerve activity of a nerve in a subject comprises applying to the nerve a low frequency stimulation signal comprising at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. In embodiments, the low frequency stimulation signal is in a range from about 0.01 Hz to about 100 Hz, preferably from about 0.01 Hz to about 30 Hz.

In other embodiments, a system and method for upregulating/stimulating nerve activity of a nerve in a subject comprises delivering to the nerve more than one stimulation active cycle to form a stimulation active phase, each stimulation active phase separated by an idle phase. The length of time of the stimulation inactive phases and/or idle provides for the ability to vary how often electrical signal treatment is applied to the nerve during an on time and allows for energy savings as compared to low frequency electrical signal therapy not having inactive phases.

In embodiments, a system and method for upregulating/stimulating nerve activity of a nerve in a subject comprises: applying to the nerve at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width, and wherein the low frequency stimulation signal is in a range from about 0.01 Hz to about 100 Hz, preferably from about 0.01 Hz to about 30 Hz.

In embodiments, a stimulation active cycle has a stimulation period comprising a pulse, and optionally, includes one or more pulse delays. The stimulation period of a pulse is based on the frequency selected and the presence of pulse delays. In embodiments, a stimulation cycle comprises at least one stimulation period; and a stimulation inactive phase.

In some embodiments, a first pulse delay occurs after the negative phase and/or a second pulse delay occurs after the positive phase. In embodiments, the first and second pulse delays are the same length. In embodiments, the length of the first and/or second pulse delay is selected to allow for a charge balanced alternating current signal to be delivered to the nerve.

In embodiments, the low frequency stimulation signal has a frequency in each period of a stimulation active cycle of at most 199 Hz, at most 150 Hz, at most 100 Hz, at most 50 Hz, at most 40 Hz, at most 30 Hz, at most 20 Hz, at most 10 Hz, at most 1 Hz, at most 0.5 Hz, at most 0.1 Hz, at most 0.05 Hz, or at most 0.01 Hz or less. In other embodiments, the frequencies range 0.01 Hz to 199 Hz, or from about 0.01 Hz to about 100 Hz, or from about 0.01 Hz to about 50 Hz, or from about 0.01 Hz to about 30 Hz, or from about 0.01 Hz to about 10 Hz. In embodiments, low frequency stimulation signals at such frequencies can upregulate nerve activity.

In embodiments, the amplitude of the signal is at least 0.01 mAmp. In other embodiments, the amplitude ranges from about 0.01 to 20 mAmps, 0.01 to 15 mAmps, 0.01 to 10 mAmps, 0.01 to 8 mAmps, or 0.01 to 5 mAmps.

In embodiments, the amplitude is at least 0.01 volt. In other embodiments, the amplitude ranges from about 0.01 to 20 volts, 0.01 to 15 volts, 0.01 to 10 volts, 0.01 to 8 volts, or 0.01 to 5 volts.

In embodiments, the on time is at least about 30 seconds. In other embodiments, the on time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include on times of varying amounts. For example, a therapy cycle can include 1 minutes of on time, 1 minute of off time, 2 minutes of on time, followed by 5 minutes of off time.

In embodiments, the off time is selected in order to allow at least partial recovery of the nerve. In embodiments, the off time may be minimized due to the presence of stimulation inactive phases and/or idle phases. In embodiments, off times are at least about 30 seconds. In other embodiments, the off time is about 30 seconds to 90 minutes, about 30 seconds to 80 minutes, about 30 seconds to 70 minutes, about 30 seconds to 60 minutes, about 30 seconds to 50 minutes, about 30 seconds to 40 minutes, about 30 seconds to 30 minutes, about 30 seconds to 20 minutes, about 30 seconds to 10 minutes, about 30 seconds to 8 minutes, about 30 seconds to 6 minutes, about 30 seconds to 4 minutes, about 30 seconds to 2 minutes, about 30 seconds to 1 minute, or about 30 seconds to 0.5 minute. In embodiments, a therapy cycle can include off times of varying amounts. For example, a therapy cycle can include 1 minute of on time, 1 minute of off time, 2 minutes of on time, followed by 5 minutes of off time.

In embodiments, the stimulation cycle comprises more than one stimulation period, each stimulation period comprising a pulse and may or may not contain pulse delays; and a stimulation inactive phase. In some embodiments, the stimulation inactive phase is longer than the stimulation period. In embodiments, the length of the stimulation inactive phase can vary between each stimulation period.

In embodiments, the stimulation period is about 0.01 seconds to about 100 seconds. In embodiments, a stimulation inactive phase is about 100 seconds or less, about 50 seconds or less, or about 10 second or less, or about 5 seconds or less, or about 1 second or less, or about 0.1 seconds or less, or about 0.01 seconds or less. In embodiments, the stimulation period is at least about 0.01 seconds up to 100 seconds, 0.01 seconds up to 50 seconds, 0.01 seconds up to 10 seconds, 0.01 seconds up to 5 seconds, or 0.01 seconds up to 1 second, or 0.01 seconds up to 0.5 seconds, or 0.01 seconds up to 0.2 seconds, or 0.01 seconds up to 0.1 seconds.

In embodiments, the stimulation inactive phase is in a ratio to the pulse of about 1000 to 1, 500 to 1, 100 to 1, 50 to 1, 10 to 1, 5 to 1, 3 to 1, or 1 to 1. In embodiments, the stimulation inactive phase is at least about 0.01 seconds, or about 0.02, or about 0.03 seconds. In embodiments, the stimulation inactive phase is at least about 0.01 seconds up to about 100 seconds, about 0.1 seconds up to about 100 seconds, or about 1 second up to about 100 seconds, or about 5 seconds up to about 100 seconds, or about 10 seconds up to about 100 seconds, or about 20 seconds up to about 100 seconds, or about 40 seconds up to about 100 seconds, or about 60 seconds up to about 100 seconds, or about 80 seconds up to about 100 seconds.

In embodiments, the stimulation inactive phase is about 0.01 seconds to about 100 seconds. In embodiments, a stimulation inactive phase is about 100 seconds or less, about 50 seconds or less, or about 10 second or less, or about 5 seconds or less, or about 1 second or less, or about 0.1 seconds or less, or about 0.01 seconds or less. In embodiments, the stimulation inactive phase is at least about 0.01 seconds up to about 100 seconds, about 0.01 seconds up to about 50 seconds, about 0.01 seconds up to about 10 seconds, about 0.01 seconds up to about 5 seconds, or about 0.01 seconds up to about 1 second, or about 0.01 seconds up to about 0.5 seconds, or about 0.01 seconds up to about 0.2 seconds, or about 0.01 seconds up to about 0.1 seconds.

In embodiments, the frequency is at most 199 Hz, at most 150 Hz, at most 100 Hz, at most 50 Hz, at most 40 Hz, at most 30 Hz, at most 20 Hz, at most 10 Hz, at most 1 Hz, at most 0.5 Hz, at most 0.1 Hz, at most 0.05 Hz, or at most 0.01 Hz or less.

In embodiments, multiple stimulation periods can be administered in a single stimulation cycle. In other embodiments, the application of the low frequency stimulation signal includes multiple stimulation cycles.

In embodiments, the low frequency stimulation signal is continuous, having multiple stimulation cycles with optional stimulation inactive phases but without idle phase. In other embodiments, the low frequency stimulation signal is pulsatile, having multiple stimulation phases and at least one idle phase, each of the multiple stimulation phase comprising two or more stimulation cycles and optional stimulation inactive phases.

In other embodiments, a system and method for upregulating/stimulating nerve activity of a nerve in a subject comprises: applying a low frequency electrical signal to the nerve, wherein the stimulation signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width. In embodiments, the stimulation signal further comprises at least one stimulation active phase, wherein each of the at least one stimulation active phase comprises two or more stimulation cycle, and wherein each of the at least one stimulation active phase is separated by an idle. In embodiments, the stimulation inactive phase is longer than the stimulation active phase. In embodiments, the stimulation inactive phase can vary in time between each stimulation active phase.

In embodiments, the stimulation active phase is at least about 10 seconds. In embodiments, the stimulation active phase is about 10 seconds to about 30 minutes, about 10 seconds to about 25 minutes, about 10 seconds to about 20 minutes, about 10 seconds to about 15 minutes, about 10 seconds to about 10 minutes, about 10 seconds to about 5 minutes, about 10 seconds to about 1 minute, about 10 seconds to about 30 seconds, or about 20 seconds to about 30 minutes, about 30 seconds to about 30 minutes, about 40 seconds to about 30 minutes, about 50 seconds to about 30 minutes, about 1 minute to about 30 minutes, about 5 minutes to about 30 minutes, about 10 minutes to about 30 minutes, about 15 minutes to about 30 minutes, about 20 minutes to about 30 minutes, about 25 minutes to about 30 minutes.

In embodiments, the stimulation active phase comprises at least 2 to 100 stimulation cycles, at least 2 to 90, at least 2 to 80, at least 2 to 70, at least 2 to 60, at least 2 to 50, at least 2 to 40, at least 2 to 30, at least 2 to 20, at least 2 to 10, at least 2 to 5, or at least 2 to 4 stimulation cycles.

In embodiments, the idle phase is in a ratio to the stimulation active phase of about 200 to 1, 180 to 1, 140 to 1, 100 to 1, 60 to 1, 20 to 1, 10 to 1, 5 to 11, to 1, 2 to 1, 3 to 1, 5 to 1, 10 to 1, 20 to 1, 60 to 1, 100 to 1, 140 to 1, 180 to 1, or 200 to 1. In embodiments, the idle phase is at least 10 seconds. In embodiments, the idle phase is 10 seconds to 30 minutes, 10 seconds to about 30 minutes, about 10 seconds to about 25 minutes, about 10 seconds to about 20 minutes, about 10 seconds to about 15 minutes, about 10 seconds to about 10 minutes, about 10 seconds to about 5 minutes, about 10 seconds to about 1 minute, about 10 seconds to about 30 seconds, or about 20 seconds to about 30 minutes, about 30 seconds to about 30 minutes, about 40 seconds to about 30 minutes, about 50 seconds to about 30 minutes, about 1 minute to about 30 minutes, about 5 minutes to about 30 minutes, about 10 minutes to about 30 minutes, about 15 minutes to about 30 minutes, about 20 minutes to about 30 minutes, about 25 minutes to about 30 minutes.

In yet other embodiments, a system and method for upregulating/stimulating nerve activity of a nerve in a subject comprises: applying to the nerve a low frequency stimulation signal to the nerve during an on time, wherein the low frequency stimulation signal comprises a first pattern and a second pattern which differ from one another. In embodiments, the first pattern comprises at least one stimulation cycle. In other embodiments, the first pattern comprises more than one stimulation active phase, wherein each stimulation active phase comprises more than one stimulation cycle, and each stimulation active phase is separated by an idle phase. In embodiments, the second pattern comprises at least one stimulation cycle. In embodiments, the second pattern comprises more than one stimulation active phase, wherein each stimulation active phase comprises more than one stimulation cycle, and each stimulation active phase is separated by an idle phase.

In yet other embodiments, a system and method for upregulating/stimulating nerve activity of a nerve in a subject comprises: applying to the nerve a low frequency stimulation signal to the nerve during an on time, wherein the electrical signal comprises a first pattern comprising at least one stimulation cycle; and a second pattern comprising more than one stimulation active phase, wherein each stimulation active phase comprises more than one stimulation cycle, and each stimulation active phase is separated by an idle phase, wherein the first and second patterns have a different amplitude and/or different on times. In embodiments, the stimulation cycle comprises at least one stimulation period and a stimulation inactive phase, each of the at least one stimulation period comprising a pulse and optionally, a pulse delay, wherein each stimulation period has a frequency of about 0.01 Hz to 199 Hz.

In embodiments, the low frequency stimulation signal has a frequency of a period which comprises a charge recharge phase and may have pulse delays, wherein the frequency is at most 199 Hz, at most 150 Hz, at most 100 Hz, at most 50 Hz, at most 40 Hz, at most 30 Hz, at most 20 Hz, at most 10 Hz, at most 1 Hz, at most 0.5 Hz, at most 0.1 Hz, at most 0.05 Hz, or at most 0.01 Hz or less. In other embodiments, the frequencies range 0.01 Hz to 199 Hz, or from about 0.01 Hz to about 100 Hz. or from about 0.01 Hz to about 50 Hz, or from about 0.01 Hz to about 30 Hz, or from about 0.01 Hz to about 10 Hz.

In embodiments, the amplitude of the signal is at least 1 mAmp. In other embodiments, the amplitude ranges from about 0.01 to 20 mAmps, 0.01 to 15 mAmps, 0.01 to 10 mAmps, 0.01 to 8 mAmps, or 0.01 to 5 mAmps.

In embodiments, the amplitude is at least 1 volt. In other embodiments, the amplitude ranges from about 0.01 to 20 volts, 0.01 to 15 volts, 0.01 to 10 volts, 0.01 to 8 volts, or 0.01 to 5 volts.

In any of the systems and methods described herein, application of a low frequency stimulation signal can be initiated or terminated using a ramp up and/or ramp down of amplitude and/or pulse width and/or frequency. In embodiments, such ramp up and ramp down times are useful to minimize sensations or discomfort from application of an electrical signal to a nerve. In embodiments, a ramp up includes multiple pulses, each pulse has an increasing increment of amplitude and/or an increasing increment of pulse width and/or a decreasing increment of frequency. In embodiments, a ramp down includes multiple pulses, each pulse has a decreasing increment of amplitude and/or a decreasing increment of pulse width and/or a decreasing increment of frequency.

In embodiments, the low frequency stimulation signal comprises an abrupt start of pulses, or a ramp up of current/voltage amplitude, or a ramp up of frequency, or a ramping up of pulse widths, or combination thereof at or near initiation of applying the low frequency stimulation signal.

In embodiments, the low frequency stimulation signal comprises an abrupt end of pulses, or a ramp down of current/voltage amplitude, or a ramp down of frequency, or a ramping down of pulse widths, or combination thereof at or near termination of applying the low frequency stimulation signal.

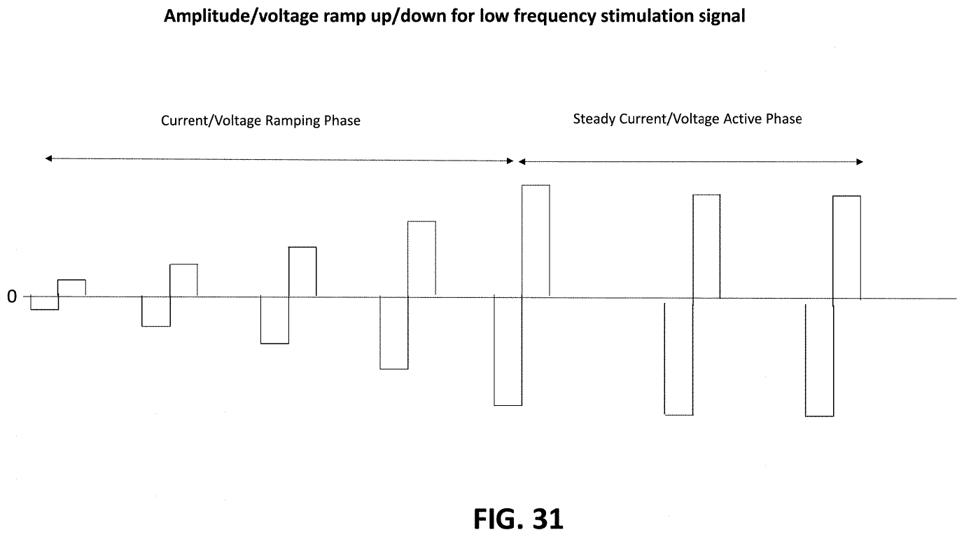

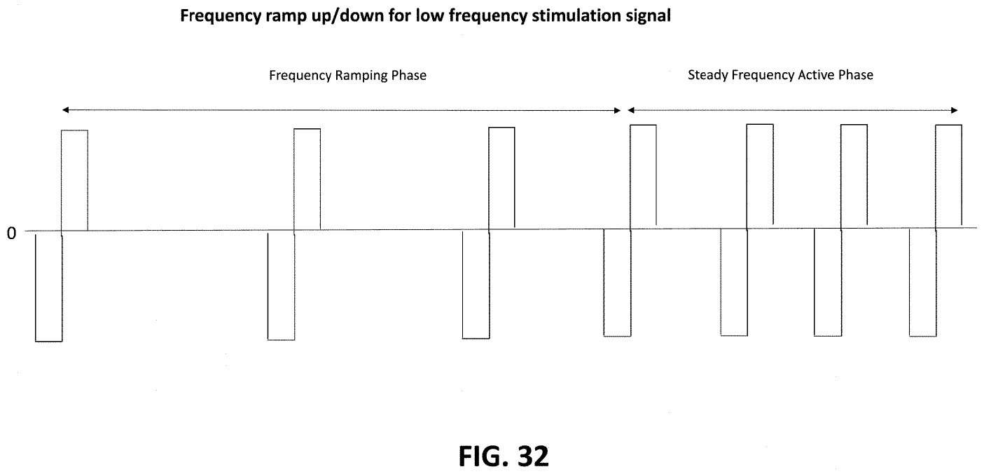

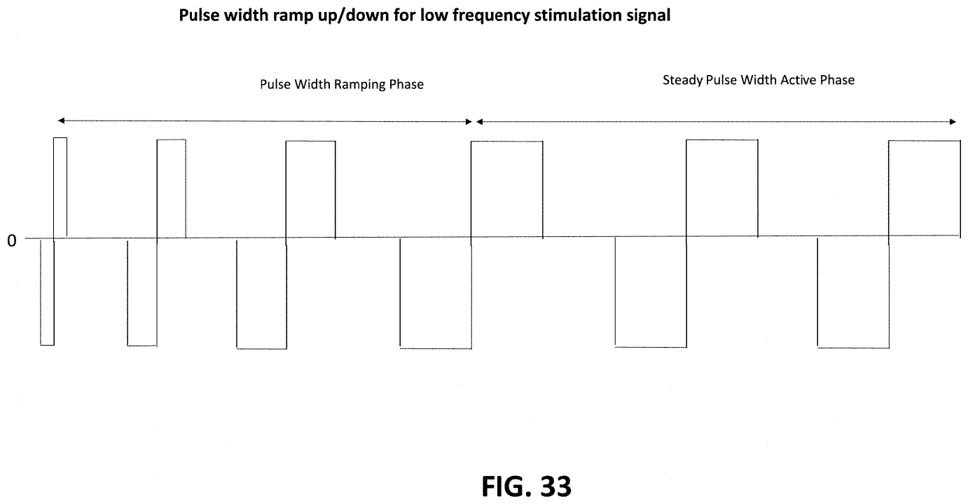

In embodiments, the low frequency stimulation signal comprises a ramping up phase at or near the initiation of the low frequency stimulation signal. FIG. 31 is an example of ramping up amplitude/voltage following the initiation of the low frequency stimulation signal. The signal starts off with a ramping phase comprising pulses having lower current amplitude/voltage, and each pulse has an increasing increment of amplitude/voltage, until the signal reaches a steady phase or steady state of amplitude/voltage of the pulses. FIG. 32 is an example of ramping up frequency following the initiation of the low frequency stimulation signal. Likewise, the signal starts off with a ramping phase comprising pulses having lower frequency, and each pulse has an increasing increment of frequency, until the signal reaches a steady phase of frequency of the pulses. FIG. 33 is an example of ramping up frequency following the initiation of the low frequency stimulation signal. Likewise, the signal starts off with a ramping phase comprising pulses having lower pulse width, and each pulse has an increasing increment of pulse width, until the signal reaches a steady phase of pulse width of the pulses.

In other embodiments, the low frequency stimulation signal comprises a ramping down phase at or near the termination of the low frequency stimulation signal. Although not shown in any figure of the present disclosure, the ramping down phase is similar to ramping up phase in principle, and can be appreciated by a person having ordinary skill in the art.

In embodiment, the low frequency stimulation signal comprises both a ramping up phase and a ramping down phase. In embodiments, the low frequency stimulation signal comprises a combination of concurrent ramping up/down of amplitude/voltage, and/or pulse width, and/or frequency, the principle of which is demonstrated in FIGS. 23 and 24.

In embodiments, the low frequency stimulation signal comprise a ramping up/down phase of about 10 seconds to about 15 minutes, or from about 10 seconds to about 10 minutes, or from about 10 seconds to about 5 minutes, of from about 10 seconds to about 1 minute, or from about 10 seconds to about 30 seconds, or from about 20 seconds to about 15 minutes, or from about 30 seconds to about 15 minutes, or from about 1 minute to about 15 minutes, or from about 5 minutes to about 15 minutes, or from about 10 minute to about 15 minutes.

In embodiments, the ramp up or ramp down of the low frequency stimulation signal is linear or non-linear.

4. Methods and Systems for Regulating Nerve Activity by Combination of Downregulation and Upregulation

In some aspects, the present disclosure relates to methods and systems for regulating nerve activity of a subject by combining a high frequency electrical signal applied to a nerve or an organ and a low frequency stimulation signal applied to a separate nerve or a separate organ. The high frequency signal has parameters to downregulate or block nerve activity, and the low frequency stimulation signal has parameters to upregulate or stimulate nerve activity.

In some embodiments, the present disclosure relates to a method and system for regulating nerve activity of a subject comprising applying a first electrical signal to a first nerve/organ and applying a second electrical signal to a second nerve/organ, wherein the first electrical signal downregulates nerve activity and has a frequency from about 200 Hz to about 100 kHz, or from about 200 Hz to about 80 kHz, or from about 200 Hz to about 60 kHz, or from about 200 Hz to about 40 kHz, or from about 200 Hz to about 20 kHz, or from about 200 Hz to about 10 kHz, or from about 200 Hz to about 5,000 Hz, or from about 200 Hz to about 2,500 Hz, or from about 200 Hz to about 1,500 Hz, or from about 200 Hz to about 1,000 Hz, and wherein the second electrical signal upregulates nerve activity and has a frequency from about 0.01 Hz to 199 Hz, or rom about 0.01 Hz to about 150 Hz, or from about 0.01 Hz to about 100 Hz, or from about 0.01 Hz to about 50 Hz, or from about 0.01 Hz to about 30 Hz, or from about 0.01 Hz to about 20 Hz, or from about 0.01 Hz to about 10 Hz. In embodiments, the first electrical signal and the second electrical signal are applied concurrently or simultaneously.

In embodiments, the first electrical signal comprises at least one microsecond cycle and optionally a microsecond inactive phase, wherein each of the at least one microsecond cycle comprises at least one period, each of the at least one period comprising a pulse comprising a charge recharge phase, the pulse having a pulse width, and wherein the second electrical signal comprises at least one stimulation cycle, wherein each of the at least one stimulation cycle comprises at least one stimulation period, each of the at least one stimulation period comprising a pulse and optionally a stimulation inactive phase, wherein the pulse comprises a cathodic and/or anodic phase and optionally a pulse delay, the pulse having a pulse width.

In embodiments, the first electrical signal further comprises at least one millisecond active phase, wherein each of the at least one millisecond active phase comprises at least one microsecond cycle, and wherein each of the at least one millisecond active phase is separated by a millisecond inactive phase. In embodiments, the second electrical signal further comprises at least one stimulation active phase, wherein each of the at least one stimulation active phase comprises at least one stimulation active cycle, and wherein each of the at least one stimulation active phase is separated by an idle.

In embodiments, the first electrical signal is low duty cycle of about 75% or less, or preferably 50% or less.

In embodiments, the pulse width of the first electrical signal is from about 10 microseconds to about 500 microseconds. In embodiments, the pulse width of the second electrical signal is from about 50 microseconds to about 10,000 microseconds.

In embodiments, the microsecond inactive phase of the first electrical signal is from about 0 to about 10,000 microseconds. In embodiments, the stimulation inactive phase of the second electrical signal is from about 0.01 to about 100 seconds.

In embodiments, the first electrical signal and the second electrical signal each independently has an on time of about 30 seconds to about 30 minutes. In embodiments, the second electrical signal has an on time of about 30 seconds to about 90 minutes, or from about 30 seconds to about 60 minutes, or from about 30 seconds to about 30 minutes.

In embodiments, the on time of the second electrical signal is about the same as the on time of the first electrical signal. In embodiments, the on time of the second electrical signal is substantially longer than the on time of the first electrical signal.

In embodiments, the first electrical signal and the second electrical signal each independently has an off time of about 30 seconds to about 30 minutes. In embodiments, the second electrical signal has an off time of about 30 seconds to about 90 minutes, or from about 30 seconds to about 60 minutes, or from about 30 seconds to about 30 minutes.

In embodiments, the on time of the second electrical signal is about the same as the off time of the first electrical signal. In embodiments, the off time of the second electrical signal is substantially longer than the off time of the first electrical signal.

In embodiments, the first electrical signal and the second electrical signal each independently has a current amplitude in a range from about 0.01 mAmps to about 20 mAmps. In embodiments, the first electrical signal and the second electrical signal each independently has a voltage in a range from about 0.01 volts to about 20 volts. In embodiments, the current amplitude/voltage of the first electrical signal is about the same as the current amplitude/voltage of the second electrical signal. In embodiments, the current amplitude/voltage of the first electrical signal is substantially longer than the current amplitude/voltage of the second electrical signal.

In embodiments, the second electrical signal comprises an abrupt start of pulses, or a ramp up of current/voltage amplitude, or a ramp up of frequency, or a ramping up of pulse widths, or combination thereof. In embodiments, the ramp up is at or near the initiation of applying the second electrical signal. In embodiments, the ramp down is at or near the termination of the second electrical signal. In embodiments, upon initiation of applying the second electrical signal, the ramp up time lasts until the second electrical signal reaches a steady state of amplitude/voltage/frequency/pulse width. In other embodiments, the ramp down starts after a steady state of amplitude/voltage/frequency/pulse width, and lasts until the termination of applying the second electrical signal. In embodiments, the ramp up/down time is about the same as the time of the steady state of amplitude/voltage/frequency/pulse width. In embodiments, the ramp up/down time is substantially shorter than the time of the steady state of amplitude/voltage/frequency/pulse width. In embodiments, the ramp up/down time is substantially longer than the time of the steady state of amplitude/voltage/frequency/pulse width.

In embodiments, the ramp up or ramp down time of current/voltage amplitude, frequency, or pulse widths of the second electrical signal is from about 10 seconds to about 15 minutes.

In embodiments, the ramp up or ramp down of the second electrical signal is linear or non-linear.

In embodiments, the first nerve and the second nerve are independently from a nerve selected from the group consisting of the vagus nerve, anterior vagus nerve, posterior vagus nerve, hepatic branch of vagus nerve, celiac branch of vagus nerve, renal nerve, renal artery, sympathetic nerves, baroreceptors, glossopharyngeal nerve.

In embodiments, the first nerve and the second are different.

In embodiments, the first organ and the second organ are selected from the group of duodenum, jejunum, ileum, small bowel, colon, stomach, esophagus, liver, spleen, pancreas, and combinations thereof.

In embodiments, the first electrode is placed on a nerve and the second electrode is place on an organ.

5. In Some Embodiments, the Present Methods and Systems Relate to Treating a Subject Having a Disease or Disorder Selected from the Group Consisting of Obesity, Overweight, Pancreatitis, Dysmotility, Bulimia, Gastrointestinal Disease with an Inflammatory Basis, Ulcerative Colitis, Crohn's Disease, Low Vagal Tone, Gastroparesis, Diabetes, Prediabetes, Type II Diabetes, Chronic Pain, Hypertension, Gastroesophageal Reflux Disease, Peptic Ulcer Disease and Combinations Thereof. Methods and Systems for Treating Disorder of Blood Glucose

In some aspects, the present disclosure relates to methods and systems for treating disorder of blood glucose or diabetes by combination of a high frequency signal and a low frequency stimulation signal described herein, the high frequency signal and the low frequency stimulation signal applied to separate nerves or nerve branches/fibers or organ.

In some embodiments, the present disclosure relates to a method for treating a condition associated with impaired glucose regulation of a subject in need thereof comprising applying a first electrical signal to one or more hepatic branches of a vagus nerve, or the anterior trunk of the vagus nerve cranial to the hepatic branch, of the subject and applying a second electrical signal to one or more celiac nerve branch of the vagus nerve, or the posterior trunk of the vagus nerve cranial to the celiac branch, of the subject, wherein the first electrical signal downregulates nerve activity and has a frequency of about 200 Hz to about 100 kHz, and wherein the second electrical signal upregulates nerve activity and has a frequency of about 0.01 Hz to 199 Hz, and wherein the first electrical signal is low duty cycle of about 75% or less. In embodiments, the first electrical signal is a high frequency signal or a high frequency low duty cycle signal according to the present disclosure. In embodiments, the second electrical signal is a low frequency stimulation signal according to the present disclosure. In embodiments, the first electrical signal and the second electrical signal are applied concurrently or simultaneously. In embodiments, the first electrical signal and the second electrical signal are applied at different/separate times. In embodiments, the first electrical signal and the second electrical signal are applied in a coordinated fashion.

In embodiments, the first electrical signal further comprises at least one millisecond active phase, wherein each of the at least one millisecond active phase comprises at least one microsecond cycle, and wherein each of the at least one millisecond active phase is separated by a millisecond inactive phase. In embodiments, the second electrical signal further comprises at least one stimulation active phase, wherein each of the at least one stimulation active phase comprises at least one stimulation active cycle, and wherein each of the at least one stimulation active phase is separated by an idle.

In embodiments, the first electrical signal is low duty cycle of about 75% or less, or preferably 50% or less.

In embodiments, the pulse width of the first electrical signal is from about 10 microseconds to about 500 microseconds. In embodiments, the pulse width of the second electrical signal is from about 50 microseconds to about 10,000 microseconds.

In embodiments, the microsecond inactive phase of the first electrical signal is from about 0 to about 10,000 microseconds. In embodiments, the stimulation inactive phase of the second electrical signal is from about 0.01 to about 100 seconds.

In embodiments, the first electrical signal and the second electrical signal each independently has an on time of about 30 seconds to about 30 minutes.

In embodiments, the first electrical signal and the second electrical signal each independently has a current amplitude in a range from about 0.01 mAmps to about 20 mAmps. In embodiments, the first electrical signal and the second electrical signal each independently has a voltage in a range from about 0.01 volts to about 20 volts.

In embodiments, the second electrical signal comprises an abrupt start of pulses, or a ramp up of current/voltage amplitude, or a ramp up of frequency, or a ramping up of pulse widths, or combination thereof at or near initiation of applying the second electrical signal.

In embodiments, the ramp up or ramp down time of current/voltage amplitude, frequency, or pulse widths of the second electrical signal is from about 10 seconds to about 15 minutes, or from about 10 seconds to about 10 minutes, or from about 10 seconds to about 5 minutes, or from about 10 seconds to about 1 minute, or from about 10 seconds to about 30 seconds, or from about 30 seconds to about 15 minutes, or from about 1 minute to about 15 minutes, or from about 5 minutes to about 15 minutes, or from about 10 minutes to about 15 minutes.

In embodiments, the ramp up or ramp down of the second electrical signal is linear or non-linear.

A dual vagus nerve bio-electronic modulation technique by combining blockage and stimulation of separate vagus nerves or vagus nerve branches/fibers in a subject provides an effective solution to treating diabetes or disorder of blood glucose. It was surprisingly found from the animal studies (shown in Examples) that the dual vagus nerve modulation technique could effectively enhance glycemic control in both rat and pig models of T2DM. Comparatively, either standalone HFAC downregulation of the vagus nerve or standalone stimulation of vagus nerve does not show the same level of therapeutic effect. The dual vagus nerve bio-electronic modulation technique will offer therapeutic benefit in human patients with T2DM.

As an exemplary embodiment of treating disorder of blood glucose, the present method comprises applying a high frequency low duty signal to the hepatic branch of a vagus nerve (or equivalently any segment of the vagus nerve central to the hepatic branching point) to downregulate the nervy activity or block conduction, and concurrently applying a low frequency electrical signal to the celiac branch of the same vagus nerve (or equivalently any segment of the vagus nerve central to the celiac branching point) to upregulate the nerve activity or stimulate conduction. It was surprisingly found that concurrent blockade of the hepatic branch of the vagus nerve with simultaneous stimulation of the celiac branch of the vagus nerve significantly improved glycemic control in animal models, compared with either blockage alone or stimulation alone (discussed in Examples). Without wishing to be bound by a particular theory, it is believe that blocking the hepatic branch via the high frequency signal may decrease glucose release from the liver and decreasing insulin resistance, while the concurrent stimulation of the celiac branch via the low frequency signal the may increase the release of insulin into the blood.

In some aspects, the present disclosure relates to a method or system for treating a condition associated with impaired glucose regulation of a subject in need thereof comprising applying a first electrical signal to one or more nerve of the subject and applying a second electrical signal to one or more nerve of the subject in a coordinated fashion.

In some embodiments, a system for treating a condition associated with impaired blood glucose regulation comprises: an implantable neuroregulator: at least one first electrode electrically connected to the implantable neuroregulator and adapted to be placed on one or more hepatic nerve branch of a vagus nerve, or the anterior trunk of the vagus nerve cranial to the branching point of the hepatic branch, of a subject; at least one second electrode electrically connected to the implantable neuroregulator and adapted to be placed on one or more celiac nerve branch of the vagus nerve, or the posterior trunk of the vagus nerve cranial to the branching point of the celiac branch, of the subject: and a blood glucose sensor configured to measure the blood glucose of the subject and convey a blood glucose value to the system, wherein the implantable neuroregulator comprises a microprocessor, wherein the microprocessor is configured to independently deliver a first electrical signal to the first nerve branch through the first electrode and deliver a second electrical signal to the second nerve branch through the second electrode, wherein the first electrical signal has parameters to downregulate nerve activity and the second electrical signal has parameters to stimulate nerve activity, and wherein the first electrical signal has a frequency of about 200 Hz to about 100 kHz, wherein the second electrical signal has a frequency of about 0.01 Hz to 199 Hz, and wherein the microprocessor is configured to apply a coordinated change to the first electrical signal and/or the second electrical signal in response to the blood glucose value.

In some embodiments, the blood glucose sensor is operatively independent from but integrated to the system. In other embodiments, the blood glucose sensor is electrically connected to the system. In yet other embodiments, the blood glucose sensor is electrically connected to the neuroregulator. In yet other embodiments, the blood glucose sensor is in wireless communication with the system.

In some embodiments, a method for regulating nerve activity of a subject comprises (1) concurrently applying a high frequency signal that blocks nerve activity to one or more hepatic branches, or the anterior trunk of the vagus nerve cranial to the branching point of the hepatic branch, of a vagus nerve of the subject and applying a low frequency stimulation signal to one or more celiac nerve branches, or the posterior trunk of the vagus nerve cranial to the branching point of the celiac branch, of the vagus nerve of the subject; (2) measuring the blood glucose of the subject by a glucose sensor to obtain a glucose value; (3) applying coordinated changes to the first and/or the second signals by tuning the parameters thereof depending on or in response to the glucose value indicated by the glucose sensor measurement.

In some embodiments, the coordinated change is selected from the group of stopping the first signal while keeping the second signal continuous, stopping the first signal while increasing the frequency of the second signal, decreasing the first signal while keeping the second signal constant, decreasing the first signal while stopping the second signal, decreasing the first signal while increasing the frequency of the second signal.

As an exemplary example, if blood glucose of a subject falls to an unsafe hypoglycemic level (e.g., in a range about 30 to about 55 mg/dL), as measured by a glucose sensor (e.g., an implantable glucose sensor or a wireless glucose sensor), coordinated changes to the blocking and stimulation signals can be applied with the intent to increase blood glucose to a safe level (e.g., at or above about 70 mg/dL) according to Table 2 shown below. For example, applying changes to stop the high frequency blocking signal and keep the low frequency stimulation signal continuous. In some embodiments, the coordinated change comprises changing the high frequency blocking signal into a low frequency signal by reducing the frequency to below 199 Hz, and terminating the low frequency stimulation signal. A user can make any change to any signals by tuning the parameters thereof through the therapy system. Alternatively, the therapy system can be programed to automatically change the therapy signals in response to the blood glucose value indicated by the glucose sensor.

TABLE 2
Coordinated changes of the high frequency blocking signal and the low frequency
stimulation signal to recover the blood glucose level to a safe level.

| Glucose value indicated by the | High frequency signal | Low frequency stimulation |
|---|---|---|
| glucose sensor | applied to a first nerve | signal to a second nerve |
| When glucose value is in a unsafe | Stop | Continuous |
| level, e.g., 30-55 mg/dL | | |
| When glucose value is in a unsafe | Stop | Increase in Frequency (from |
| level, e.g., 30-55 mg/dL | | 1-9 Hz to 10 to 199 Hz) |
| When glucose value is in a unsafe | Constant | Increase in frequency (from |
| level, e.g., 30-55 mg/dL | | 1-9 Hz to 10 to 199 Hz |
| When glucose value is in a unsafe | Lower frequency (from | Constant |
| level, e.g., 30-55 mg/dL | above 199 Hz to a range | |
| | of 199 Hz to 0.01 Hz) | |
| When glucose value is in a unsafe | Lower frequency (from | Stop |
| level, e.g., 30-55 mg/dL | above 199 Hz to a range | |
| | of 199 Hz to 0.01 Hz) | |
| When glucose value is in a unsafe | Lower frequency (from | Increase frequency (from |
| level, e.g., 30-55 mg/dL | above 199 Hz to a range | 1-9 Hz to 10 to 199 Hz) |
| | of 199 Hz to 0.01 Hz) | |

### EXAMPLES

### Example 1—Experiments to Test the Ability of High Frequency Low Duty Cycle Electrical Signals

Experiments to test the ability of low duty cycle as illustrated in the exemplary embodiments in the figures to block a nerve as compared to high duty cycle were conducted on an isolated rat vagus nerve. Compound action potentials (hereinafter CAP) were elicited with a bipolar hook stimulation electrode and recorded with a bipolar hook recording electrode positioned at approximately 16 mm away from the stimulation electrode. A third bipolar hook electrode that delivered high frequency alternating current (HFAC) algorithms (either high duty cycle or low duty cycle) was positioned between the stimulation and recording electrode.

The amplitude of the CAP was measured for 1 min before the application of HFAC and within 1 second following cessation of HFAC. Baseline was calculated by taking the average amplitude of the CAPs for 1 min prior to the delivery of HFAC. Block was measured by taking the CAP amplitude following HFAC and dividing it by the baseline CAP amplitude.

With a HFAC amplitude in the range of about 6 mA it was determined that all of the exemplary low duty cycle electrical signal patterns as shown in FIGS. 6-10 blocked the nerve to the same degree as the high duty cycle algorithm depicted in FIG. 5. (data not shown)

Conduction along the vagus nerve did not recover immediately following HFAC for high or low duty cycle electrical signal treatment. The time of recovery was on the order of about 5 minutes. For high and low duty cycle electrical signal treatment recovery times were similar.

### Example 2—the Duration and Intensity of HFAC Influences the Degree and Recovery of Nerve Conduction Block

Example 2 was carried out to investigate various HFAC parameters for the induction of prolonged conduction block while minimizing energy requirements would allow the development of smaller devices. Electrically-evoked compound action potentials (CAP, neurograms) from isolated vagus nerves was used to assess the influence of HFAC amplitude and duration on degree of carry-over of axonal conduction block after cessation of HFAC. A family of current-effect curves was generated at different HFAC durations to test if degree of block, measured with-in 1 second following the cessation of 5,000 Hz, and recovery time progressively increased with larger HFAC amplitudes and durations.

Method

Vagus Nerve Isolation

All experimental procedures were approved by the Institutional Animal Care and Use Committee at the University of Minnesota and performed on adult male Sprague-Dawley rats (225-375 g, n=17). Rats were euthanized with an overdose of isoflurane before an incision was made in the lower neck to expose the cervical vagus nerve. The ribcage was also removed to expose the thoracic vagus. Oxygen-saturated synthetic interstitial fluid (SIF; containing (in mM) 123 NaCl, 3.5 KCl, 0.7 MgSO₄, 2.0 CaCl₂), 9.5 Na gluconate, 1.7 NaH₂PO₄, 5.5 glucose, 7.5 sucrose, and 10 HEPES; pH 7.45) was introduced to the exposed cervical and thoracic cavities. The left and right cervical/thoracic vagus nerves were dissected from neck to the level of the heart. Vasculature and fat were further dissected to remove excess tissue from the nerve. Following excision, the nerves were placed in ice-cold oxygenated SIF. Electrophysiological study was initiated within 10 minutes of the transfer of the nerve from chilled oxygenated SIF to the recording chamber. The second nerve remained in the ice-cold oxygenated SIF until experimentation was finished on the first nerve (typically about 90 minutes).

Electrophysiology

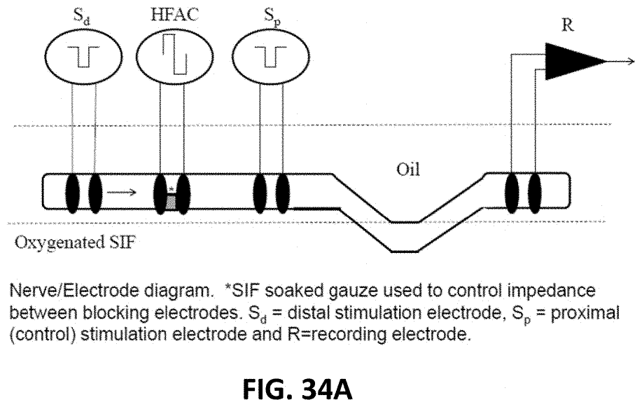

Excised nerves were positioned in a recording chamber on three, and in some cases four, sets of bipolar hook electrodes and suspended in mineral oil. The recording chamber was suspended inside a hot water bath held at 34° C. The electrode arrangement was similar to that in Waataja et al. with an electrode delivering HFAC positioned between stimulation (“distal” electrode) and recording electrodes. In some experiments a “proximal” stimulating electrode was positioned between the blocking electrode and the recording electrode (FIG. 34(a)). The stimulation and recording electrodes consisted of pairs of platinum/iridium and Ag/AgCl wire (0.01-0.015 inch diameter), respectively. The electrode delivering HFAC consisted of a pair of platinum-iridium ribbon wires (0.02 inch thickness; 0.05 inch width) in a hook configuration which cradled the nerve (180 degrees of contact). Blocking electrodes were separated by 2 mm. A piece of oxygenated SIF soaked gauze was placed between the pair of blocking electrodes to maintain a similar impedance between nerves/experiments. This also increased availability of oxygen and nutrients to the nerve at the site of the blocking electrode. A layer of oxygenated SIF below the mineral oil provided a grounding path between the blocking and recording electrode, or in some cases between the proximal stimulation electrode and the recording electrode. Temperature measurements were taken inside the recording chamber to assure the nerve was exposed to a constant temperature of 34° C. The recording chamber consisted of an inner and outer chamber. The outer chamber contained a thermostatically controlled heating element submerged in water. The outer chamber housed an inner chamber holding the vagus nerve in mineral oil with an underlying layer of SIF. The vagus nerve was electrically activated through the stimulation electrodes with monophasic (negative) pulses generated by a constant current stimulus isolation unit (A385, World Precision Instruments, Sarasota, Fla., USA) driven by a pulse generator (Isostim A320, World Precision Instruments, Sarasota, Fla., USA) at 1 Hz. Typical stimulus durations were 0.1-0.5 ms and amplitudes 0.5-4.5 mA. In cases where a proximal stimulation electrode was used, two similar stimulation paradigms were implemented, as described above, with a 200 ms delay between stimulations. Stimulus-evoked nerve signals were led from recording electrodes to the headstage of a differential amplifier (DAM 80, 10,000× gain, typical bandpass of 10 Hz to 3 kHz, World Precision Instruments, Sarasota, Fla., USA) and referenced to a Ag/AgCl pellet in the underlying SIF. The resulting signal was led in parallel to an oscilloscope and a data acquisition system (Power 1401 with Spike 2, Cambridge Electronic Design, Cambridge, UK). In some recordings a rolling average of 30 data points was utilized to filter out high frequency noise.

High Frequency Alternating Current (HFAC)

Generation of the HFAC signal was done through a proprietary device provided by ReShape Lifesciences Inc. (San Clemente, Calif., USA). The signal was a 5000 Hz bi-phasic constant current square waveform consisting of a charge component and a recharge component (FIG. 34(b)). The charge and recharge waveform components were of opposite polarity and were generated from the same current source so the current in each component was matched. The waveform was measured throughout 5000 Hz delivery to ensure that the current of each waveform component was within specification and the voltage of each waveform component matched within specification. Any deviation outside of specification would result in termination of the signal. However, this did not occur during this study. The output of the pulse generator was not capacitively coupled but rather employed a proprietary method for charge balance. Shorting periods (10 μs) were incorporated as part of the duty cycle of each of the charge and recharge waveform components. During these shorting periods, the electrodes were short-circuited together to remove any charge remaining after application of the waveform.

The pulse generator has been measured for direct current (DC) and consistently met a<1 uA leakage current specification; typically <50 nA. The average impedance at 5000 Hz (1-10 mA, biphasic square wave) between HFAC electrodes placed on a vagus nerve in mineral oil was 1700+/−52 Ohms. HFAC was applied for 10, 30, 60 or 120 seconds. HFAC was delivered at 1, 3, 5, 8 and 10 mA current amplitudes. The HFAC amplitudes reported are measured from the base to the peak of the waveform.

Measurements and Analyses

Isolated vagus nerves were electrically activated, and compound action potential (CAP) waveforms were recorded over atypical distance of 16-24 mm. In experiments which included a proximal stimulation electrode the typical nerve length was 26 mm. Conduction distance was measured as the shortest distance between the stimulation and recording electrodes, latency was measured from onset of stimulus artifact to time of initial peak of CAP waveforms, and peak conduction velocity (CV) of each waveform was estimated as distance/latency (m s⁻¹). Waveform amplitudes were measured from peak to peak.

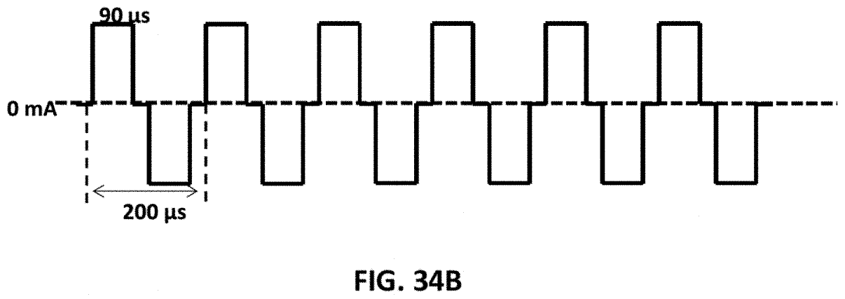

Before testing the effects of HFAC, CAP waveforms were optimized by adjusting stimulus duration and amplitude. Typically, CAP waveform amplitudes at 1.5-2.0 times stimulus threshold were established as baseline measures. Vagus nerve CAPs had A6 and C waveforms characterized by CVs of 3.7-9.4 m s⁻¹ and 0.52-0.86 m s⁻¹, respectively. An example of a CAP before HFAC can be seen on the top trace of FIG. 35.

Experiments were only conducted after CAP amplitudes remained constant for at least 15 minutes and the condition that the CAP had a static CV; indicating the temperature of the nerve had reached an equilibrium. Typically the CV increased slightly during the first 5 minutes in the heated recording chamber before reaching a steady state. From all 20 possible combinations of duration and amplitude, one combination was chosen at random to be applied first on a given nerve. Following full recovery, and a 5 min resting period, another combination was chosen at random and applied to the nerve. This pattern was repeated no more than 6 times on one nerve: or if there was a lack of full recovery following the application of HFAC. With this protocol it happened that a similar condition may have been tested on the same rat or nerve more than once, however; the study did not end until all conditions were tested on at least 4 rats. The total number of times an individual condition was tested was greater or equal to 5.

CAP recordings were obtained for 1 minute before HFAC (baseline) and within 1 second following cessation of HFAC, until recovery was evident. Degree of block was measured as the CAP amplitude immediately following termination (within 1 second) of the HFAC, unless otherwise specified, divided by the average baseline values. Full nerve block was defined as a depression of the CAP waveform to 5% of its baseline value measured at the first evoked CAP following HFAC, unless otherwise specified. The CAP was considered fully recovered when the CAP waveform returned to 95% of its baseline amplitude. Measurements from the oscilloscope were used to determine nerve block and recovery. The time course of recovery was determined from continuous data generated by the data acquisition system. In proximal stimulation control experiments CAPs were evaluated during the application of 5000 Hz. Using Spike 2 software a low pass filter (1750 Hz threshold and a 125 Hz transition gap) was applied to the digitized neurogram. Visual inspection was made and there were no major changes in CAP morphology with these settings. To quantify the degree of block during recovery, the area between the normalized time course recovery curve and baseline was calculated.

Graphing was performed with SigmaPlot (Systat Software, Chicago, Ill., USA). Statistical analyses were performed by SAS Software-Version 9.3 (SAS Institute, Inc., Cary, N.C., USA.). All data are presented as mean±SEM. A linear mixed effects model with random intercept was used to model outcomes. The random intercept was used to model repeated measurements on the same nerve. The fixed effects of duration, amplitude and duration by amplitude interaction were included in the models. Least square means based on model results were used to further explore potential interaction effects. The Tukey-Kramer method was used for the pairwise comparisons of the model based least square means. An alpha level of 0.05 or less was considered significant. Each parameter was used on a subset of nerves from at least 4 of the 17 rats studied.

Results

Degree of Nerve Block Following 5000 Hz was Dependent on HFAC Amplitude and Duration

All trials on an individual nerve, with various combinations of HFAC amplitude and durations, used a randomization procedure. There was not a progressive increase of HFAC amplitude or duration on individual nerves. A particular combination of HFAC amplitude and duration may have occurred at the first trial, anywhere between the first and last trial or the last trial on a nerve; per the randomization procedure. Block was measured within 1 second following the cessation of 5,000 Hz. At a fixed HFAC duration, current amplitudes of 1, 3, 5, 8 and 10 mA were delivered to the nerve at 5,000 Hz. The amplitude of the A6 and C waves decreased progressively with increasing HFAC current amplitudes (FIG. 35). There were no apparent changes in peak CV. The A6 wave was more sensitive to HFAC amplitude then the C wave (FIG. 35). At 60 seconds HFAC duration, the A6 wave was fully blocked at 8 mA, whereas it required 10 mA to fully block the C wave.

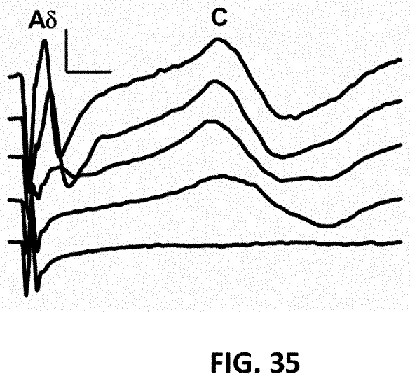

To test the effect of HFAC duration on the degree of nerve block, a family of current-effect curves was created at different HFAC durations for A6 and C waves. The HFAC durations tested were 10, 30, 60 and 120 seconds. For statistical comparisons, only C wave data obtained for durations of 30 seconds and greater were evaluated. For A6 waves, HFAC durations of 10 seconds produced a current-effect curves with increasing waveform attenuation as HFAC current amplitudes were increased (FIG. 36(a)). At an HFAC duration of 10 seconds, full blockade could only be produced at an HFAC amplitude of 10 mA (1 of 5 nerves tested). On average 10 mA with a 10 seconds duration produced a 74%±9% attenuation of the A6 wave.

There was a considerable leftward shift in the A6 wave current-effect curves as HFAC durations progressed from 10 to 30 seconds. Complete block was achieved at 10 mA at 30 seconds duration. For C waves, attenuation was achieved at HFAC durations of at least 30 seconds and amplitudes greater than 5 mA (FIG. 36(b)).

The Aδ wave current-effect curve for 60 second HFAC duration was similar to that of 30 seconds (no significant difference at all amplitudes tested), except full block was observed at 8 mA. For the C wave at 10 mA, there was a significantly greater block with 60 seconds duration (95±4% decrease) than at 30 seconds (44±15% decrease).

The greatest leftward shift in the Aδ wave current-effect curves, was at 120 second HFAC duration (FIG. 36(a)). The Aδ wave was significantly more sensitive to 3 mA at 120 second HFAC duration than 60 second HFAC duration, and full block was achieved at 5 mA. The current-effect curves for the C wave were similar at 60 and 120 second HFAC durations (no significant difference at all amplitudes tested).

Considering all combinations of HFAC intervals and amplitudes, there was a significant effect of interactions between HFAC duration and amplitude on the degree of block of Aδ and C waves (test of fixed effects). For HFAC amplitudes greater than 1 mA there was a significant difference in degree of block across HFAC durations for the Aδ wave (test of effects). For HFAC amplitudes greater than 5 mA there was a significant difference in degree of block across HFAC durations for the C wave (tests of effects). Similar degrees of block at the same current amplitude and duration were observed regardless of the trial number between nerves.

The general shapes of the current-effect curves for Aδ and C waves were very different. For Aδ waves at HFAC durations greater than 10 seconds, the current-effect curves had large initial negative slopes that progressively decreased as HFAC current amplitudes increased (FIG. 36(a)). The best fit for this behavior was an exponential function of the form:

CAP Amplitude=α*e^{-β*(HFAC Amplitude)}

where α and β are constants chosen to give the best fit. The best fit R² values were 0.99, 0.98 and 0.97 for 30, 60 and 120 seconds, respectively. Slopes of the current-effect curves for C waves became more steep as current amplitudes were increased (FIG. 36(b)). The best fit curve for this behavior was a polynomial function of the form:

CAP Amplitude=−a*(HFAC Amplitude)²+b(HFAC Amplitude)−c

where a, b and c are constants chosen to give the best fit. The best fit R² values were 0.99, 0.95 and 0.99 for 30, 60 and 120 seconds, respectively.

Recovery Time of CAP Amplitude was Dependent on the Amplitude and Duration of HFAC

The time required for both Aδ and C waves to recover following conduction blockade was dependent on HFAC current amplitude at all durations tested. There was a gradual and roughly linear increase in recovery times of Aδ waves as HFAC current amplitude was increased (FIG. 37(a)). Recovery of C waves followed the same trend as Aδ waves, with increased recovery times as HFAC current amplitudes were increased (FIG. 37(b)).

Recovery times of Aδ waves were dependent on HFAC duration. For HFAC amplitudes above 1 mA there was a significant difference in recovery time across HFAC durations (test of effects). For example, it took the Aδ wave 6.7±0.19 minutes to recover at 10 mA with 30 second HFAC duration compared to 2.9±0.7 minutes at 10 mA with 10 seconds HFAC duration (FIG. 37(a)). The same tendency of longer HFAC durations extending recovery time was observed for 60 and 120 second with apparent leftward shifts in their current-recovery curves compared to 30 seconds and less duration. For the C wave at 8 and 10 mA HFAC current amplitudes, there was a significant difference in recovery time across HFAC durations (test of effects, FIG. 37(b)).

Recovery times for Aδ waves did not differ significantly (between 6.7 and 7.2 minutes, FIG. 37(a)) at combinations of HFAC duration and current amplitude (10 mA for 30 seconds, 8 mA for 60 seconds and 5 mA for 120 seconds, (FIG. 36(a)) that produced full conduction block. For the C wave, the same magnitude of block was observed at 10 mA for both 60 and 120 second HFAC durations (FIG. 36(b)). However, recovery time after 60 second HFAC duration (3.6±1.3 minutes, FIG. 37(b)) appeared to be about half of that observed with 120 second HFAC duration (8.1±1.8 minutes, FIG. 37(b)).

Not every nerve recovered following HFAC. Lack of recovery was defined as a static CAP amplitude less than 95% of its initial amplitude for about 20 mins following cessation of HFAC. Full recovery was evident in the majority off all combinations tested with the exception of 10 mA at 60 and 120 second HFAC duration for the C wave. With these combinations approximately 33% of nerves tested experienced a partial recovery (˜50 to 80% steady amplitude for 20 minutes following cessation of 5000 Hz) but did not meet the 95% wave amplitude to be considered fully recovered. Lack of recovery was observed at these higher current amplitudes/durations regardless if the HFAC signal applied prior to any other HFAC signal or after multiple applications of HFAC. Similar recovery times occurred at the same current amplitude and duration regardless of the trial number between nerves.

Degree of Sustained Block During Recovery

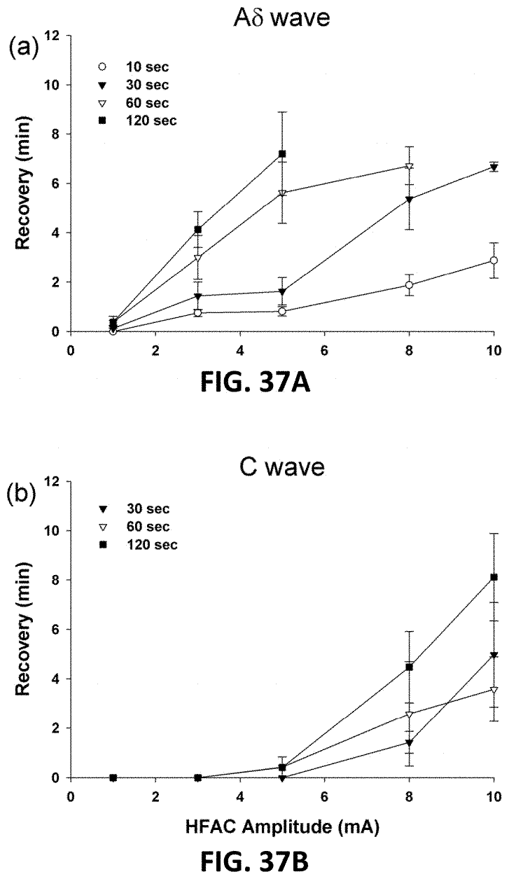

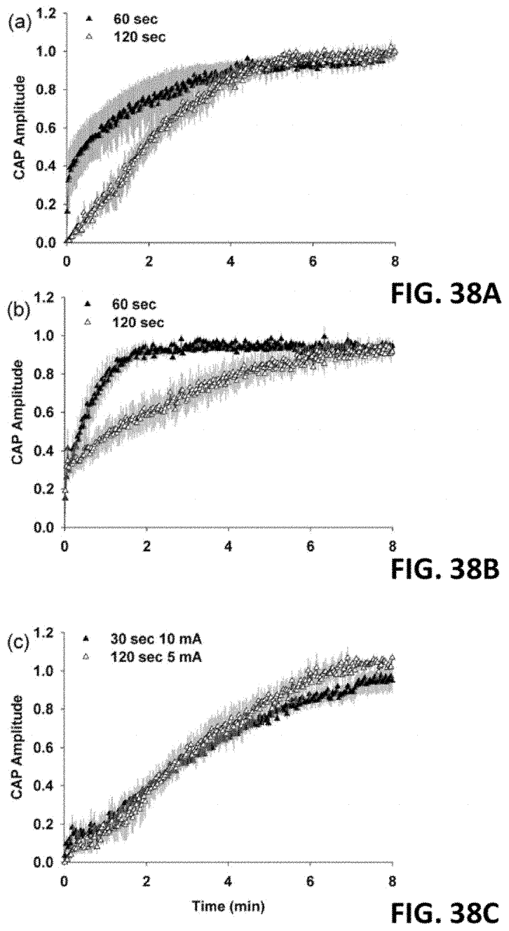

Sole reliance on descriptions of the degree of block immediately following the cessation of HFAC and recovery time is insufficient for the assessment of the degree of sustained block during recovery. For example, two different combinations of HFAC amplitude and duration may produce similar recovery times and degrees of block immediately following the cessation of HFAC. However, the initial degree of nerve block may not predict the dynamics of recovery, including how long a substantial blockade of the nerve will persist following the conclusion of HFAC delivery. To better estimate overall degree of sustained block during recovery, we first plotted CAP amplitude at various time points following HFAC delivery ((FIG. 38(a)) for each combination of HFAC duration and intensity. We next calculated the “area above the curve” for plots of CAP recovery following HFAC as a measure of the degree of block during recovery. These values for “area” (area unit=time*(1−CAP amplitude)) were then plotted against HFAC amplitude for different durations ((FIG. 39).

In general, the degree of sustained block during recovery correlated well with the initial degree of block and recovery time for most combinations of HFAC amplitudes and durations. However, there were some deviations from this pattern. For example, 5 mA of HFAC delivered for 60 or 120 seconds produced a relatively similar degree of initial block of Aδ waves (86±7% and 100±0%, respectively, (FIG. 36(a)). The times to recovery from block under the same conditions were also reasonably similar (5.7±1.3 and 7.2±1.7 minutes, respectively, FIG. 37(a)). However, the amount of sustained block of Aδ waves during recovery with 5 mA for 60 seconds HFAC duration (33±10 area units) was only 35% of that produced by 120 second HFAC at 5 mA (93±8 area units; FIG. 39(a)). This difference can be appreciated by comparing the time course of recovery for both conditions (FIG. 38(a)).

For the C wave, a substantial difference in sustained block during recovery was observed following 10 mA of HFAC for 60 seconds (22±5 area units) compared to 120 seconds (71±22 area units; FIG. 39(b)). The degree of initial nerve block following HFAC was similar under these same conditions (95±5% and 86±11%, respectively). A large part of the difference in amount of block during recovery can be attributed to different times of recovery (3.6±1.3 minutes and 8.1±1.8 minutes, FIG. 37(b)) for the 60 and 120 second HFAC durations. However, the kinetics of recovery also differs. The initial slope of the recovery curve following 60 seconds of HFAC was much steeper than that following 120 seconds duration (FIG. 38(b)).

A similarity in the sustained block during recovery between different combinations of HFAC amplitudes and durations was that when full block was achieved the degree of block during recovery was similar for the Aδ wave. Complete block immediately following HFAC was achieved for the Aδ wave at all of the following combinations: 30 seconds 10 mA, 60 seconds 8 mA and 120 second 5 mA. For all of these combinations the degree of block during recovery was similar (FIG. 39(a)). FIG. 37(c) demonstrates the similarities of the recovery kinetics of 10 mA with 30 seconds HFAC duration and 5 mA with 120 seconds HFAC duration for the Aδ wave.

It should be noted that same degree of block during recovery (as well as initial degree of block or time of recovery) was not dependent on testing on the 1^{st} nerve or the 2^{nd} (which was resting in the ice-cold oxygenated SIF during the first nerve experimentation).

Proximal Stimulation Control Experiments

It has been demonstrated that activation of fibers can occur during the application of HFAC (Kilgore and Bhadra 2013). This begs the question that the carry-over effect we observed was due to activation-induced fatigue of the nerve. To control for this possible event, longer segments of vagus nerve were excised and an additional proximal stimulation electrode was placed between the blocking electrode and recording electrode (FIG. 34(a)), similar to Williamson et. al. (Williamson and Andrews 2005). Also, a longer segment of nerve was grounded in the underlying SIF, between the proximal electrode and recording electrode, which allowed for CAPs to be measured during the application of 5000 Hz. Using this method, HFAC durations of 30 and 120 seconds, with various current amplitudes, were chosen due to the fact that these durations induced a full and partial degree of block immediately following the cessation of 5000 Hz for both the Aδ and C wave demonstrated through earlier experiments in this study.

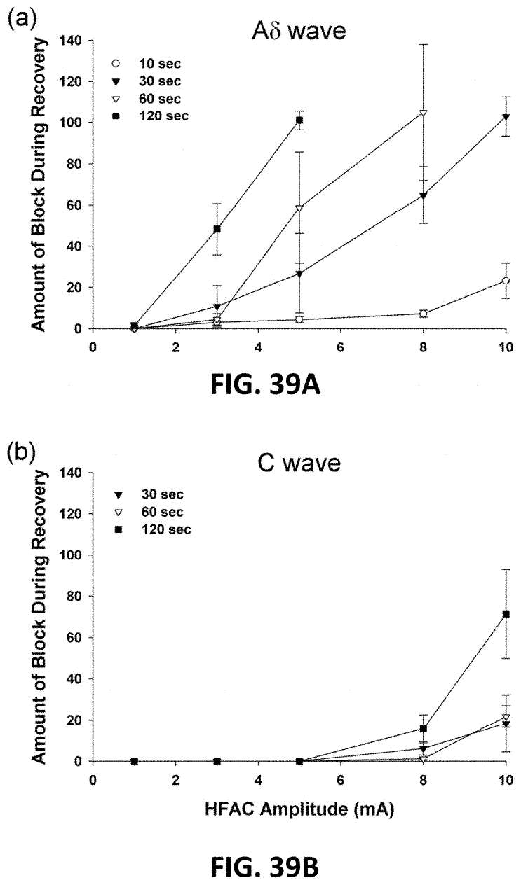

For the Aδ wave a 120 second application of HFAC at 5 mA was chosen to be tested with a proximal stimulation electrode. This was done to determine if the disappearance of the CAP elicited by the distal stimulation electrode following 5000 Hz was induced by a long duration activation-induced fatigue or conduction block under the blocking electrode. If there was a substantial long duration depression of the Aδ wave elicited by the proximal electrode (“proximal Ad wave”) during and following 5000 Hz then activation-induced fatigue would likely be the mechanism of the carry-over block. Within the first second following the initiation of HFAC there was an 90±10% decrease in the Aδ wave amplitude produced by the distal electrode (“distal Aδ wave”, FIG. 40(a)). At this time there was an 18±5% decrease of the proximal Aδ wave amplitude, likely due to activation-induced collision block at the onset of HFAC. The proximal Aδ wave reached a maximal depression of 25±11% decrease at 3 seconds following the initiation of 5000 Hz. Over time the Aδ wave produced by the distal electrode continued to decrease and reached a full block at about 30 seconds following the initiation of 5000 Hz, on average, and full block persisted for the rest of the application of HFAC. On the other hand the Aδ wave produced by the proximal increased to baseline levels by the end of the application of 5000 Hz (FIG. 40(a)). The distal Aδ wave continued to be depressed for minutes following the application of 5000 Hz with recovery evident at about 6 minutes, on average, whereas the proximal Aδ wave continued to remain at baseline amplitude during this period.

Next, proximal stimulation was utilized with a HFAC amplitude/duration combination that produced a partial block of the Aδ wave immediately following 5000 Hz, A HFAC duration of 30 seconds at a 5 mA amplitude was tested. At the first stimulation during the application of 5000 Hz the distal Aδ wave was depressed by 95±6% whereas the proximal Aδ wave was depressed by 24±7% (FIG. 40(b)). The distal Aδ wave continued to be attenuated by about 95% during the application of 5000 Hz whereas the proximal Aδ wave was depressed by about 10% on average. At the first stimulation (within 1 second) following 5000 Hz the distal Aδ wave was attenuated by 79±13% and at approximately 7 seconds, on average, following cessation of 5000 Hz there was a quick increase in the distal CAP amplitude to a 44±9% depression with a steady recovery over the course of minutes. There was no attenuation of the proximal Aδ wave following HFAC (FIG. 40(b)).

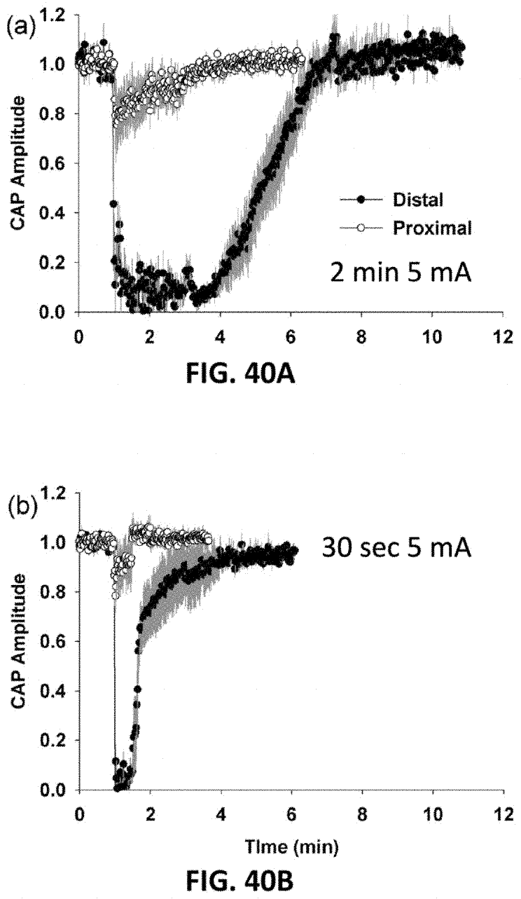

Proximal stimulation was performed using a 120 second HFAC duration at 8 and 10 mA HFAC amplitudes which was shown earlier in this study to produce a partial and a near full block of the C wave following the application of 5000 Hz, respectfully. At the first stimulation during the application of a 10 mA HFAC amplitude the distal C wave was depressed by 70±11% whereas the proximal C wave was attenuated by 14±6% (FIG. 41(a)). The amplitude of the distal C wave continued to decline and a full attenuation was observed at about 90 seconds following the initiation of HFAC, on average, which was sustained until the last stimulation during HFAC. The proximal C wave amplitude increased during the application of 5000 Hz and returned to baseline amplitude in about 40 seconds following the initiation of HFAC on average (FIG. 41(a)). Following the application of 5000 Hz the distal C wave remained approximately 95% blocked but experienced a partial quick recovery of about 20% 30 seconds following cessation of 5000 Hz in these control experiments. Following this it took minutes to for the distal C wave to recover whereas the proximal C wave remained at baseline values during this time (FIG. 41(a)).

A combination of HFAC amplitude/duration was tested which produced a partial block of the C wave following the application of 5000 Hz that was demonstrated through experiments earlier in this study. The combination tested was 120 seconds HFAC duration at an 8 mA current amplitude. For the 8 mA 120 second HFAC application there was a 33±13% decrease in the distal C wave amplitude following 1 second of HFAC (FIG. 41(b)). The C wave elicited by the proximal electrode was unchanged at this time. During the course of the HFAC application the C wave amplitude elicited from the distal electrode decreased and was depressed by 66±18% at the last stimulation during 5000 Hz whereas there was no change in amplitude of the proximal C wave at this time. Following the application of 5000 Hz the distal C wave increased in size following the first stimulation to an attenuation of 46±16%. It took minutes for the wave to recover and there was no change in the proximal C wave during this time (FIG. 41(b)).

### Example 3—Effectiveness and Efficiency of High Frequency Low Duty Cycle Electrical Signals

Methods

Similar experimental procedure as described in Example 2 were followed in Example 3.

Vagus Nerve Isolation

All experimental procedures were approved by the Institutional Animal Care and Use Committee at the University of Minnesota and performed on adult male Sprague-Dawley rats (225-375 g, n=17). The left and right cervical/thoracic vagus nerves were dissected from neck to the level of the heart and placed in ice-cold oxygenated synthetic interstitial fluid (SIF). Electrophysiological study was initiated within 10 minutes of the transfer of the nerve from SIF to the recording chamber (34° C.). The second nerve remained in the ice-cold oxygenated SIF until experimentation was finished on the first nerve (typically 90 minutes).

Electrophysiology

Excised nerves were suspended on three, and in some cases four, sets of bipolar hook electrodes in mineral oil (FIG. 34(a)). An electrode delivering HFAC was positioned between stimulation (“distal” electrode) and recording electrodes. In some experiments an additional “proximal” stimulating electrode was positioned between the blocking and the recording electrodes (FIG. 34(a)). The stimulation and recording electrodes consisted of pairs of platinum/iridium and Ag/AgCl wire (0.01-0.015 inch diameter), respectively. The electrode delivering HFAC consisted of a pair of platinum-iridium ribbon wires (0.02 inch thickness; 0.05 inch width).

The vagus nerve was electrically activated through the stimulation electrodes with monophasic (negative) pulses (typical durations of 0.1-0.5 ms and amplitudes 0.5-4.5 mA). In cases where a proximal stimulation electrode was used, two similar stimulation paradigms were implemented with a 200 ms delay between stimulations. Stimulus-evoked nerve signals were led from recording electrodes to the headstage of a differential amplifier (10,000× gain, typical bandpass of 10 Hz to 3 kHz) and referenced to an Ag/AgCl pellet in the underlying SIF. The resulting signal was led in parallel to an oscilloscope and a data acquisition system.

High Frequency Alternating Current (HFAC)

Generation of the HFAC signal was done through a proprietary device provided by ReShape Lifesciences (San Clemente, Calif.). The signal was a bi-phasic constant current square waveform at a frequency of either 5,000 Hz or 1,000 Hz. The charge and recharge waveform components were generated from the same current source so the current in each component was matched. The waveform was measured throughout HFAC delivery to ensure that the current of each waveform component was within specification and the voltage of each waveform component matched within specification. The output of the pulse generator was not capacitively coupled but rather employed a proprietary method for charge balance. Shorting periods (10 μs) were incorporated as part of the duty cycle. During these shorting periods, the electrodes were short-circuited together to remove any charge remaining after application of the waveform.

The pulse generator has been measured for direct current (DC) and consistently met a<1 uA leakage current specification; typically <50 nA. The average impedance between the blocking electrodes was 1700+/−52 Ohms.

Measurements and Analyses

Compound action potential (CAP) recordings were obtained for 1 minute before HFAC (baseline) and within 1 second following cessation of HFAC, until recovery was evident. Degree of block was measured as the CAP amplitude immediately following termination (within 1 second) of the HFAC, unless otherwise specified, divided by the average baseline values. Following recovery, the nerve was given 5 minutes to rest before another HFAC duration or amplitude was tested. 5,000 Hz induced noise was digitally low pass filtered, in some cases, at a 1750 Hz threshold and a 125 Hz transition gap.

All data are presented as mean±SEM. To test the influence of duration and current amplitude on degree of block a linear mixed effects model with random intercept was used to model outcomes. A least square means based on model results were used to further explore potential interaction effects. The Tukey-Kramer method was used for the pairwise comparisons of the model based least square means.

Results

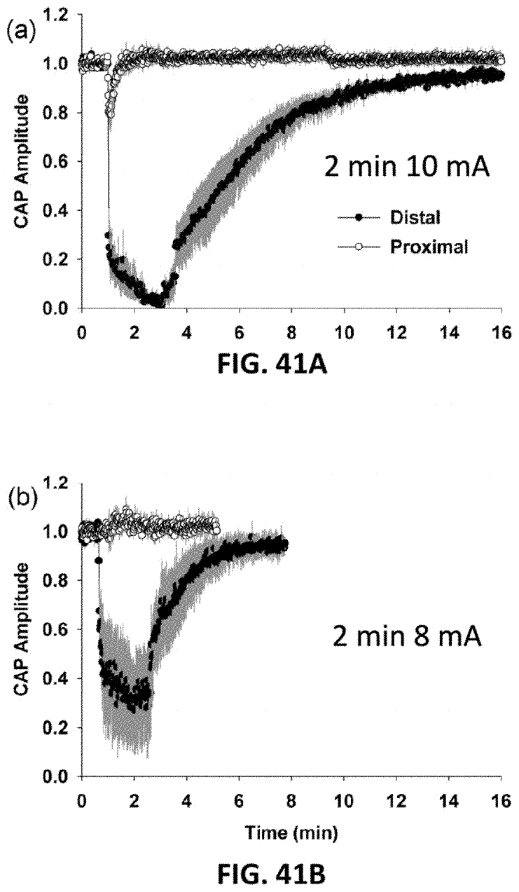

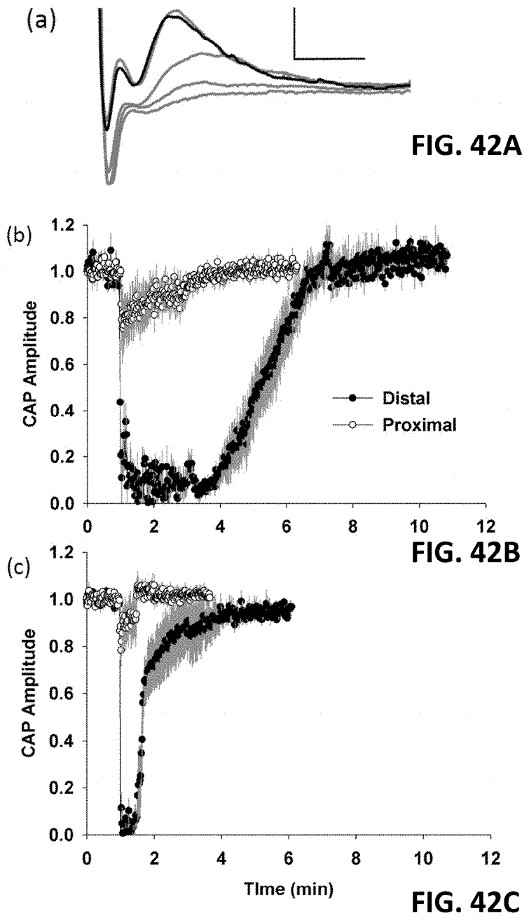

Results are shown in FIGS. 42 and 43. As can be seen, following the cessation of HFAC, conduction block persisted at the site of the blocking electrode. Considering all combinations of HFAC application durations and amplitudes, there was a significant effect of interactions between HFAC duration and amplitude on the degree of block of the CAP. For HFAC amplitudes greater than 1 mA there was a significant difference in degree of block across HFAC durations. Recovery time was significantly influenced by both the HFAC duration and amplitude.

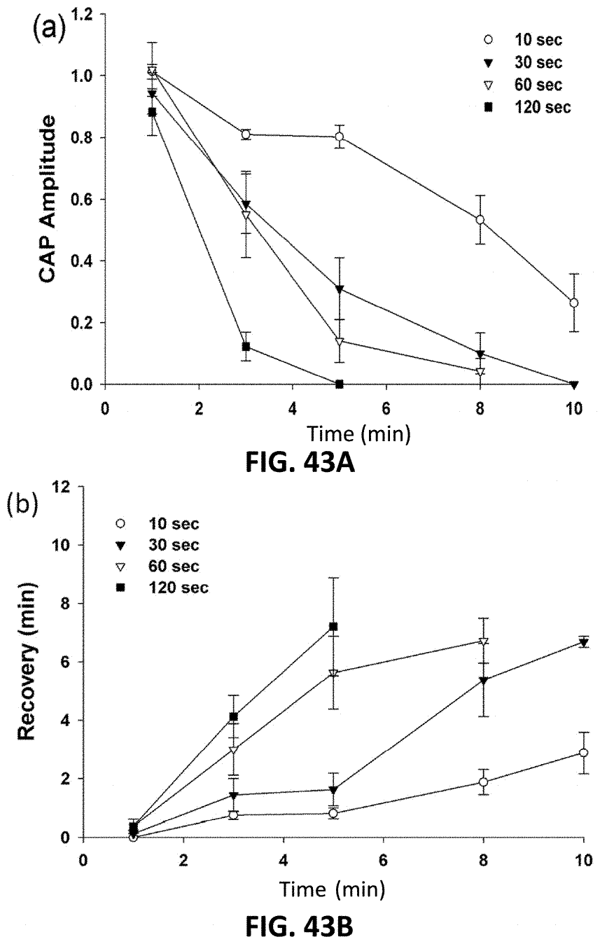

FIG. 44 shows the design of electrical signals. FIG. 44(a) shows a traditional HFAC algorithm having high duty cycle of about 90% as a control. FIG. 44(b) shows a low duty cycle HFAC algorithm comprising a 1000 Hz signal with 90 μs pulse widths incorporated 820 μs inactive periods, with a low duty cycle of about 18%. FIG. 44(c) shows a low duty cycle HFAC algorithm comprising a 1000 Hz signal with microsecond inactive periods and was interwoven with 20 millisecond inactive periods, with a low duty cycle of about 12%.

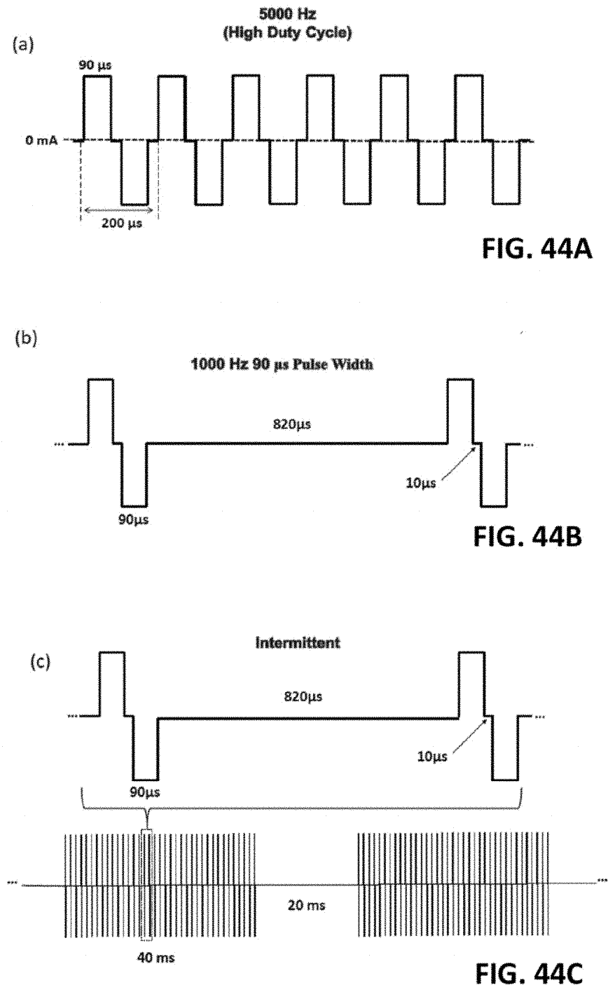

As shown in FIGS. 45(a) and 45(b), the low duty cycle HFAC signals (FIG. 44(b) and FIG. 44(c)) produced about the same degree of nerve conduction block and the time of recovery as the high duty cycle HFAC (FIG. 44(a)). These unexpected results strongly supports that lowering the duty cycle on the scale of microseconds and milliseconds could achieve a similar carry over blockade as a high duty cycle signal while significantly reduce power consumption.

### Example 4—Strength Duration for Stimulating Sub-Diaphragmatic Pig Vagus Nerve

A comparative study on mono-phasic and bi-phasic waveforms of electrical signals for stimulating Sub-diaphragmatic pig vagus nerve was carried out. This is used to determine the excitability of the nerve with different pulse types.

Strength-duration relationships were determined by varying the stimulation amplitude (max 10 mA) and duration (max 10 ms) to a threshold required to evoke CAP waveforms. Chronaxie and rheobase were determined by fitting the strength duration curve to power functions of the form:

strength=α(duration)^{-β}.

where α and β are constants chosen to give the best fit. Rheobase was defined as the stimulus strength (mA) required to elicit a threshold compound action potential waveform with 10 ms stimulus duration. To determine the chronaxie, the rheobase was doubled and the power function was solved for duration at this strength.

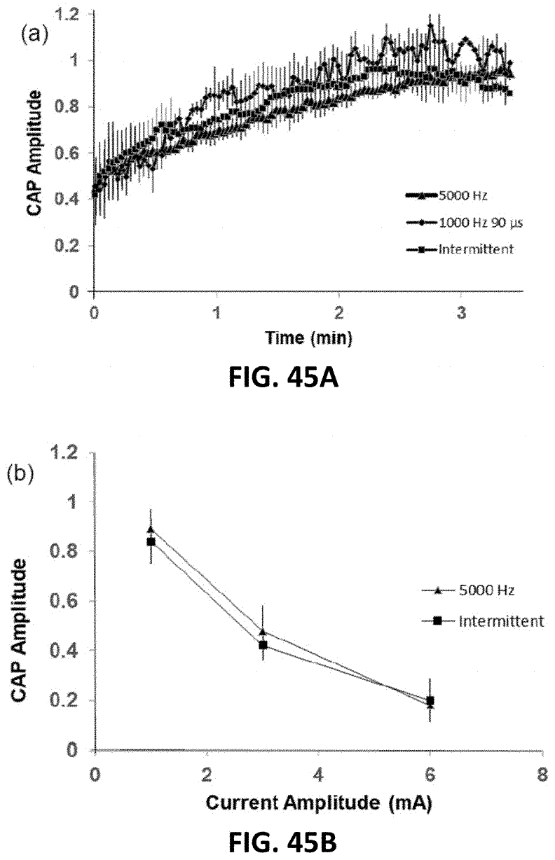

As shown in FIG. 46, the stimulation signal with a mono-phasic pulse has a relatively lower excitability of the pig vagus nerve compared with the stimulation signal with bi-phasic pulses. Accordingly, the mono-phasic stimulation signal may also have relatively longer duration strength and better energy efficiency. However, it should be noted that the excitability of the nerve may also depend on the nerve type and factors other than the waveform.

### Example 5—Combination of Downregulation and Upregulation of Rat Vagus Nerve

Method

Rat Experiments

Rat experiments were approved by the Institutional Animal Care and Use Committee at the University of Minnesota. Male ZDF rats (63 days old), or adult male Sprague Dawley control rats, were given food ad libitum (Purina #5008 for ZDF rats and Envigo 2918 for Sprague Dawley) except for an 18 hr fast prior to glucose challenge experiments. Rats were anesthetized with an intraperitoneal (IP) injection of sodium pentobarbital (40-50 mg/kg). Next, rats were placed on a heating blanket and the right jugular vein was cannulated. The depth of anesthesia was assessed periodically by testing a paw withdrawal reflex. If a reflex was observed a maintenance dose (5 mg/kg) of pentobarbital was administered IV. Next, the abdominal cavity was opened and the liver retracted. The hepatic branch of the anterior sub-diaphragmatic vagus nerve and the celiac branch of the posterior sub-diaphragmatic vagus nerve were isolated and separated from the esophagus. There were 5 experimental conditions: 1) sham operation (nerve isolation only, ZDF n=6, Sprague Dawley n=5), 2) vagotomy/stimulation (positive control, ZDF n=4, Sprague Dawley n=5), 3) HFAC/stimulation (ZDF n=4, Sprague Dawley n=5), 4) vagotomy alone (ZDF n=4) and 5) stimulation alone (ZDF n=4). In the vagotomy/stimulation group the hepatic branch was ligated, and the celiac branch was stimulated at 1 Hz. In the HFAC/stimulation group, a 5000 Hz alternating current signal was applied to the hepatic branch, and the celiac branch was stimulated at 1 Hz. In the vagotomy alone group the hepatic branch was ligated. In the stimulation alone group the celiac branch was stimulated at 1 Hz and the hepatic branch remained intact.

Stimulation and HFAC parameters consisted of the following: the celiac branch was suspended on bipolar platinum/iridium wires (0.01 inch diameter). A monophasic negative square wave with a pulse width of 4 millisecond was generated by a grass s44 stimulator (Grass Medical Instruments, Quincy, Mass., USA) which drove a stimulus isolation unit (Model A360, World Precision Instruments, Sarasota, Fla., USA) at 1 Hz. The pulse amplitude was 8 mA. For delivery of HFAC, the hepatic branch was suspended on bipolar platinum/iridium ribbon wires (0.02 inch thickness; 0.05 inch width). The electrode made a 180° contact with the nerve. The current amplitude was 8 mA.

One hour following all procedures (except for 15 min following the HFAC/stimulation procedure) a blood sample was taken from a cut end of the rat's tail. An AlphaTrak (Abbott Laboratories, North Chicago, Ill., USA) blood glucose monitor was used to measure blood glucose concentrations (mg/dL). Typical fasting glucose for ZDF rats was 200 mg/dL and 150 mg/dL for Sprague Dawley control rats. Next, an IVGTT was performed. The IVGTT consisted of an IV injection into the port of a 0.5 g/kg dose of glucose made up in 0.9% saline with a 20% weight/volume concentration. Blood glucose was then sampled for 30 min following the glucose injection. Stimulation and/or delivery of HFAC were maintained during the IVGTT. In some cases, a subsequent IVGTT was administered in the sham group and 15 min following the cessation of HFAC and stimulation in the HFAC/stimulation group.

High Frequency Alternating Current (HFAC)

A Maestro® Model 2002 neuroregulator (ReShape Lifesciences Inc., San Clemente, Calif.) was used to generate a 5000 Hz signal in all experiments involving HFAC. The signal consisted of a bi-phasic constant current square waveform consisting of a charge component and a recharge component (FIG. 45(a)).

Two Maestro® Model 2200-47E leads (ReShape Lifesciences Inc., San Clemente, Calif.) with cuff electrodes were used to deliver 5000 Hz (8 mA) in the in vivo pig experiments. The exposed electrode surface area was 10.3 mm² with an exposed electrode circumferential length of 7.3 mm and an exposed electrode width of 1.4 mm (FIG. 34(a)). The electrodes made a 180° contact with the nerve.

Analysis

To decrease the effective variability in fasting plasma glucose (FPG) between animals and to make comparisons between species, changes in glucose were normalized to baseline glucose. Baseline glucose was measured 5 min prior to the IVGTT in rats and 10 min prior to the OGTT in pigs. Percent change in glucose concentration was calculated using the following equation:

% Change=((glucose concentration at time x−Baseline glucose concentration)/(Baseline glucose concentration))*100.

The glucose response was quantified by calculating the area under the curve (AUC, % change in glucose concentration*time=area units (AU)).

The area between a line connecting two subsequent data points and the x-axis was calculated as one segment. The total number of segments following the glucose challenge was then summated. Comparisons between the condition tested and control consisted of a student's t-test with a nominal alpha level of 0.05 as considered significant. Comparisons between multiple conditions were not made. All data are presented as mean±SEM.

Results

HFAC with Concurrent Stimulation Improved Performance on an IVGTT in ZDF Rats

ZDF rats are homozygous for a non-functional leptin receptor which causes obesity and insulin resistance. Pancreatic B-cells have also been shown to fail to respond to glucose in these rats. This model was used to verify that HFAC applied to the hepatic branch of the vagus nerve with concurrent celiac branch stimulation will reversibly increase glycemic control in an animal model of T2DM. To access glycemic control an IVGTT was chosen over an oral or intra-peritoneal glucose challenge because the rat was anesthetized with its abdominal cavity exposed.

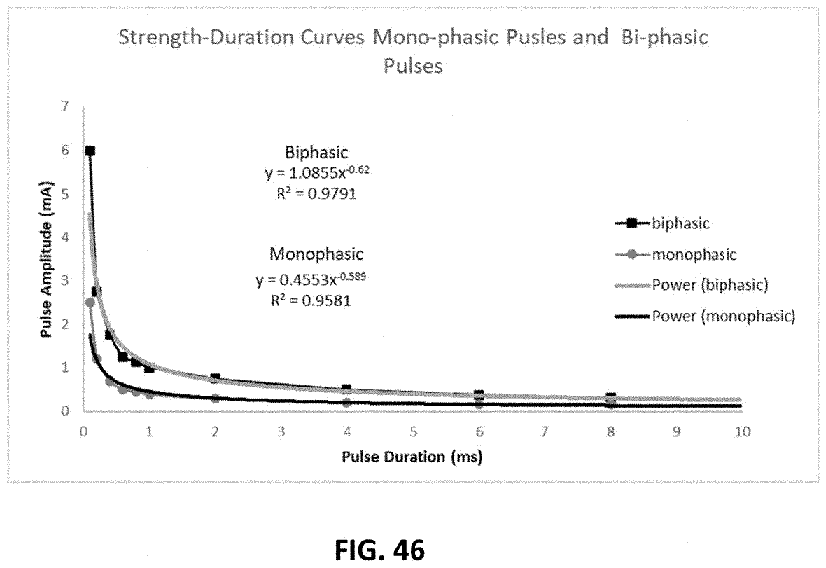

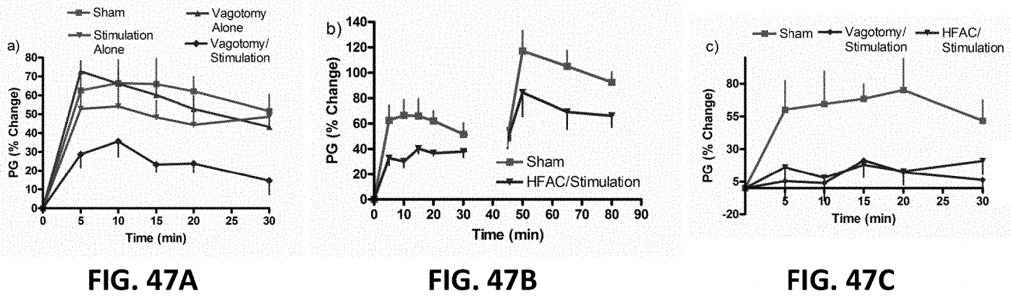

First, control experiments were performed which consisted of 4 conditions: a sham operation, a vagotomy alone, a stimulation alone and a vagotomy/stimulation positive control. One hour following these procedures an IVGTT was administered. Blood glucose increased by an average of 63±12% 5 min following the glucose injection in the sham group and remained elevated for a half hour with a partial recovery (FIG. 47(a), AUC=1543±257 AU). For the hepatic vagotomy alone and the stimulation alone groups there was no significant difference in the increase in glucose compared to sham following the challenge (FIG. 47(a), vagotomy alone AUC=1425±157 AU, stimulation alone AUC=1220±250 AU). However, there was a significant decrease in glucose levels compared to sham in the vagotomy/stimulation group following the challenge (FIG. 47(a), AUC=618±111 AU, p<0.01).

To test if HFAC applied to the hepatic branch with concurrent celiac stimulation mimicked the increased glycemic control as in the vagotomy/stimulation group, HFAC and stimulation were applied 15 min prior to and during an IVGTT. Following the IVGTT there was a significant decrease in glucose compared to sham (FIG. 47(b), AUC=898±68, p<0.05). Following cessation of HFAC/stimulation a second glucose injection induced a large increase in glucose which was non-significant, albeit slightly attenuated, to a subsequent glucose injection in the sham group (FIG. 47=7(b)). This suggests a functional recovery following cessation of HFAC/stimulation.

Experiments in control Sprague Dawley rats demonstrated a similar and significant pattern as in ZDF rats. In the sham group glucose increased by 60±22% following administration of glucose with a partial recovery at 30 min. When the celiac branch was stimulated with either a concurrent hepatic ligation or concurrent delivery of 5000 Hz there was a significant decrease in glucose following the challenge compared to sham (FIG. 47(c), sham=1704±553 AU, vagotomy/stimulation AUC=202±322 AU p<0.05, HFAC/stimulation AUC=418±140 AU, p<0.05). In all conditions for both ZDF and Sprague Dawley rats glucose remained steady for the treatment period prior to the IVGTT.

Without wishing to be bound by a particular theory, it is believed that vagus nerve stimulation alone does not offer increase in glycemic control. Celiac stimulation, or stimulation of the vagus nerve central to the celiac branching point, can cause an increase in plasma insulin however glucose is either unchanged or increased. It is possible that this is due to simultaneous pancreatic release of glucagon causing hepatic glucose release. But simultaneously blocking conduction through the hepatic branch likely attenuates the livers sensitivity to glucagon.

### Example 6—Combination of Downregulation and Upregulation of Rat Pig Nerve

Method

In Vivo Pig Experiments

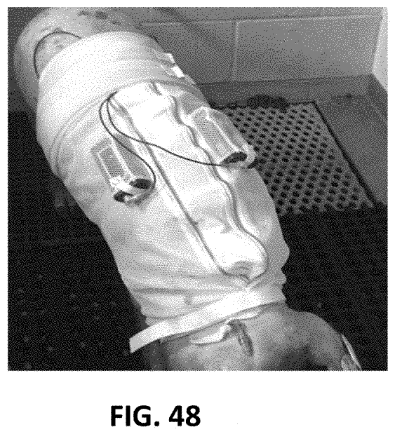

Pig experiments were approved by the Institutional Animal Care and Use Committee at North American Science Associates, Inc. (Brooklyn Park, Minn.). Adult Yucatan pigs (˜45 kg, n=6) were allowed to acclimate for 7 days following shipment from Sinclair Bio Resources (Auxvasse, Mo.). In 3 pigs a proprietary titrated dose of Alloxan was administered at Sinclair Bio Resources (Auxvasse, Mo.) to the pig via an IV injection 8 weeks prior to shipment. The pigs were monitored and fed ad libitum for 24 hours following Alloxan treatment to prevent any possible hypoglycemia due to release of insulin into the blood due to beta cell death. The pigs were not insulin dependent. Pigs were offered food twice per day (Teklad 7200, Envigo, for the non-diabetic pigs and CU Sinclair S-9 Ration, Sinclair Bio Resources, for the Alloxan treated pigs) except for an 18 hr fast prior to glucose challenges. Following acclimation pigs were trained to drink 100 mL of diet Gatorade delivered through a syringe as well as to wear a jacket to house mobile charging units during charging sessions (FIG. 48). The OGTTs consisted of oral consumption of 75 g of glucose dissolved in 100 mL of diet Gatorade. An IV port was placed in the jugular vein.

For surgical implantation of the Maestro® neuroregulators (2) and leads (4), pigs were anesthetized with Telazol/Xylazine given IM at a dose of 6 mg/kg and 1 mg/kg Xylazine. Animals were intubated and maintained on isoflurane inhalant anesthetic to effect (1.0-2.0%). Two Maestro® leads with platinum iridium cuff electrodes were placed on the anterior sub-diaphragmatic vagal trunk at the hepatic branching point and sutured onto the esophagus to deliver HFAC. A second pair of identical leads with cuff electrodes were placed on the posterior sub-diaphragmatic vagal trunk at the celiac branching point and sutured onto the esophagus to deliver a bi-phasic charge balanced pulse at 1 Hz (4 ms pulse width and 8 mA current amplitude). Each lead had a wing suture tab between the electrode tip and the connection to the neuroregulator. The wing was sutured to the stomach as a strain relief.

The electrodes were connected to leads that were tunneled to 2 Maestro® neuroregulators in a subcutaneous pocket above the ribcage on either side of the pig. The pigs were allowed to recover for 10 days following implant before pre-device initiation OGTTs were performed. To charge the neuroregulators, following HFAC/stimulation experiments, a coil was positioned over the neuroregulator above the skin layer. The coil was then connected to a Maestro® mobile charger and delivered a 6.78 MHz radio frequency signal to the implanted neuroregulator.

Isolated Pig Vagus Nerve Electrophysiology

Pig vagus nerves were harvested following euthanization. After this the esophagus with vagus nerve trunks attached was extracted from the level of the stomach to below the level of the heart. The block of tissue was then placed in ice cold oxygenated synthetic interstitial fluid ((SIF; containing (in mM) 123 NaCl, 3.5 KCl, 0.7 MgSO4, 2.0 CaCl₂), 9.5 Na gluconate, 1.7 NaH2PO4, 5.5 glucose, 7.5 sucrose, and 10 HEPES; pH 7.45). Next, both anterior and posterior nerve trunks were dissected from the esophagus at the sub-diaphragmatic gastric branches to a level below the heart and placed in ice cold oxygenated SIF.

Excised nerves were positioned in a recording chamber on four sets of bipolar hook electrodes and suspended in mineral oil. The recording chamber was suspended inside a hot water bath held at 34° C. The electrode arrangement was similar to that in Waataja et al. with an electrode delivering HFAC positioned between stimulation (“distal” electrode) and a recording electrode. The distal stimulation electrode was positioned just above the gastric branches, below the level of the diaphragm as well as the electrode delivering HFAC. The recording electrode was placed at the opposite rostral end of the nerve segment. A “proximal” stimulating electrode was positioned between the blocking electrode and the recording electrode (FIG. 34(a)). The stimulation and recording electrodes consisted of pairs of platinum/iridium and Ag/AgCl wire (0.01-0.015 inch diameter), respectively. The electrode delivering HFAC consisted of a pair of platinum-iridium ribbon wires (width of 1.4 mm) in a hook configuration which cradled the nerve (180 degrees of contact); similar to the in vivo pig experiments. Blocking electrodes were separated by 2 mm. The nerve made contact with a layer of oxygenated SIF below the mineral oil, between the proximal stimulation and recording electrodes, which helped supply oxygen/nutrients and provided a grounding path. Temperature measurements were taken inside the recording chamber to assure the nerve was exposed to a constant temperature of 34° C.

The recording chamber consisted of an inner and outer chamber. The outer chamber contained a thermostatically controlled heating element submerged in water. The outer chamber housed an inner chamber holding the vagus nerve in mineral oil with an underlying layer of oxygenated SIF. The vagus nerve was electrically activated through the stimulation electrodes with monophasic (negative) pulses generated by a constant current stimulus isolation unit (A385, World Precision Instruments, Sarasota, Fla., USA) driven by a pulse generator (Isostim A320, World Precision Instruments, Sarasota, Fla., USA) at 1 Hz. Typical stimulus durations were 0.1-0.5 ms and amplitudes 0.5-4.5 mA. The methodology for the proximal and distal stimulation was the same except the proximal electrode was activated 200 ms following the activation of the distal stimulation electrode. Stimulus-evoked nerve signals were led from recording electrodes to the headstage of a differential amplifier (DAM 80, 10,000× gain, typical bandpass of 10 Hz to 3 kHz, World Precision Instruments, Sarasota, Fla., USA) and referenced to a Ag/AgCl pellet in the underlying SIF. The resulting signal was led in parallel to an oscilloscope and a data acquisition system (Power 1401 with Spike 2, Cambridge Electronic Design, Cambridge, UK).

The 5000 Hz signal was applied for 1 min. Baseline compound action potentials (CAPs) were recorded 1 min prior to the application of the 5000 Hz signal at a rate of 1 Hz. Compound action potential amplitude was normalized to average baseline values. Following cessation of 5000 Hz a CAP was elicited within 1 second and was defined as the degree of block (or “CAP Amplitude”) on the current-effect curve.

Analysis

Methods of analysis was the same as described in Example 5.

Results

High Frequency Alternating Current Blocked Conduction Through Porcine Vagus Nerve

A 5000 Hz HFAC signal has been shown to reversibly block conduction through sub-diaphragmatic rat vagus nerve and low frequency sub-diaphragmatic vagus nerve monophasic stimulation parameters was established before. In the present example, the effect of application of 5000 Hz HFAC on larger sub-diaphragmatic swine nerves and optimal parameters for bi-phasic stimulation were investigated. To test current amplitudes required to block and stimulate the sub-diaphragmatic pig vagus nerve, electrically elicited compound action potential (CAPs) (n=5) was observed. The isolated nerve was suspended on 4 hook electrodes; a distal stimulation electrode, an electrode delivering HFAC, a control proximal stimulation electrode and a recording electrode (FIG. 34(a)).

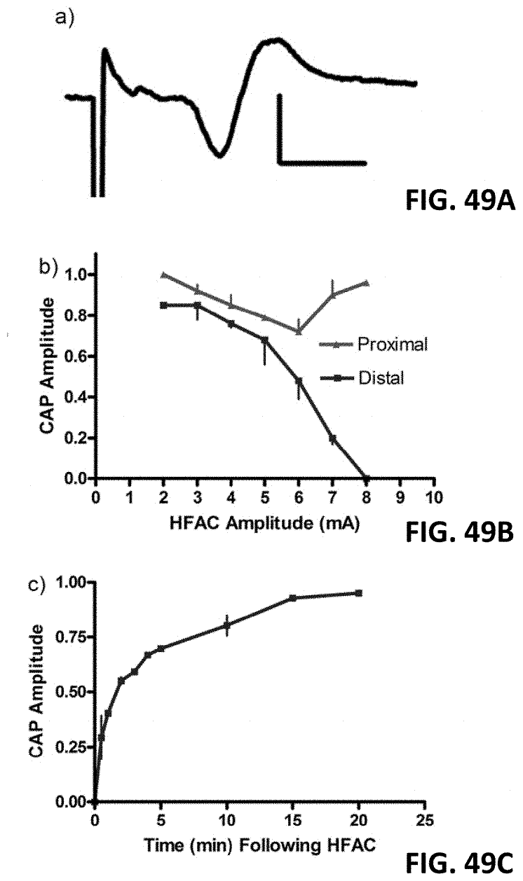

The distal and proximal stimulation electrodes both elicited a CAP (FIG. 49(a)). However, when HFAC was applied the distal CAP amplitude decreased in a current dependent manner with consistent full block at 8 mA (FIG. 49(b)). The proximal electrode was used as a control to test for repetitive firing of action potentials, which have been shown to occur with the application of HFAC. If the HFAC elicited anti- and ortho-dromic action potentials there would be collision blocks with action potentials elicited by the stimulation electrodes decreasing the amplitude of the CAPs. There was a decrease in the CAP produced by the proximal electrode in a current dependent manner at HFAC amplitudes less than 6 mA which peaked at an average of a 28% decrease (FIG. 49(b)). However, as the HFAC current amplitude was increased the proximal CAP increased to baseline values with only an average of a 4% decrease at 8 mA. Following cessation of HFAC the distal CAP amplitude returned to 90% of its baseline amplitude within 15 min, and ≥95% at 20 min, on average (FIG. 49(c)). The conduction block that persisted following the cessation of HFAC is also called a “carry-over” effect.

Stimulation of the sub-diaphragmatic pig vagus nerve was tested using a charge balanced bi-phasic square wave generated by the Maestro® neuroregulator. Maximal CAP amplitudes consistently occurred at current amplitudes of 8 mA and pulse widths of 4 ms.

HFAC with Stimulation Increased Glycemic Control in Alloxan Treated Swine

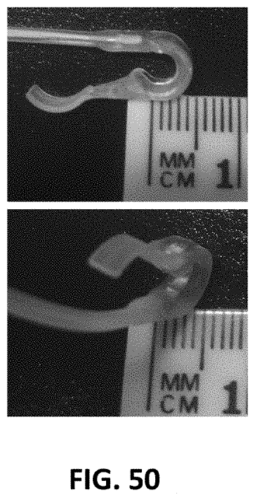

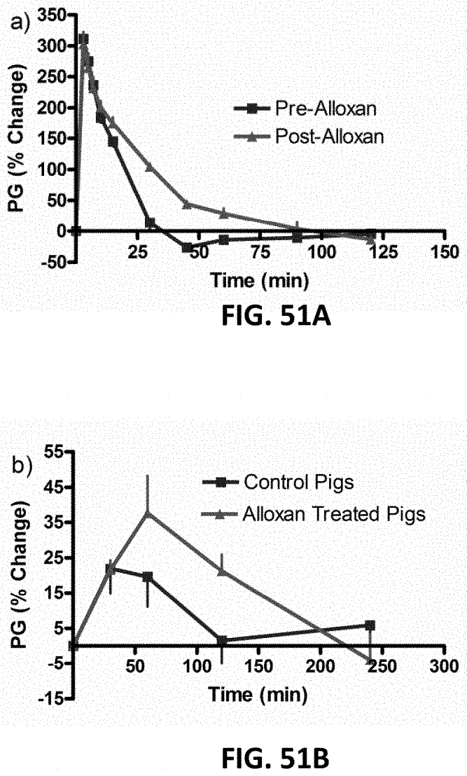

Combination of HFAC/stimulation was then tried in a chronic study in a pig model of T2DM (n=3). By a proprietary method at Sinclair Bio Resources (Auxvasse, Mo.), a titrated dose of Alloxan-induced partial ablation of beta cells. Following Alloxan pigs had decreased glycemic control but were not insulin dependent. An IVGTT was conducted prior to, and following, the Alloxan treatment which demonstrated significantly decreased glycemic control following Alloxan (FIG. 51(a), pre-Alloxan AUC=3237±362 AU, post-Alloxan AUC=7230±483 AU, p<0.001). Following acclimation from shipment, pigs were offered an OGTT. This showed a significantly decreased performance compared to non-Alloxan treated control pigs (FIG. 51(b), control pig AUC=1582±451 AU, Alloxan treated AUC=2976±519 AU, p<0.05). Next, two Maestro® neuroregulators were implanted and two electrodes placed on the anterior vagal trunk at the branching point of the hepatic nerve and two electrodes place on the posterior vagal trunk at the branching point of the celiac nerve and sutured to the esophagus. The anode and cathode electrodes delivering HFAC were the same dimensions and configuration as used in the isolated pig vagus nerve electrophysiology study (FIG. 50).

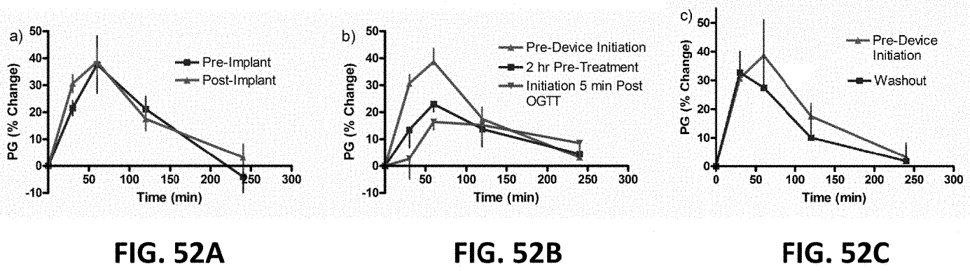

Following 10 days of recovery from surgery 3 OGTTs were conducted (2 days separation) with the devices off (pre-device initiation OGTT). The results from the OGTTs were consistent and similar to the pre-implant OGTT (FIG. 52(a), AUC pre-implant=2976±519 AU, AUC post-implant=2600±579 AU). Fasting plasma glucose was also similar pre- and post-implant (pre-implant FPG=84±6 mg/dL, post-implant FPG=86±4 mg/dL). Next, the devices were turned on with 5000 Hz HFAC delivered to the hepatic branch and a 1 Hz biphasic signal delivered to the celiac branch for 2 hrs. This caused a significant 16% decrease in FPG (72±2 v 86±4 mg/dL, p<0.01). Next an OGTT was performed 2 hrs following, and during, HFAC/stimulation which demonstrated a significant decrease in glucose relative to pre-device initiation OGTTs (FIG. 52(b), pre-device initiation AUC=2600±579 AU, 2 hr pretreatment AUC=1617±220 AU p<0.01).

Combination of HFAC/stimulation was carried out to test if it would increase glycemic control following the initiation of an OGTT. In this experiment the HFAC/stimulation was initiated 5 min following the start of the OGTT. This treatment demonstrated a significant decrease in glucose (AUC=1277±249 AU) compared to pre-device initiation (FIG. 52(b), p<0.001). A control washout OGTT demonstrated that following 3 days of cessation of HFAC/stimulation, glycemic control returned/recovered to pre-device initiation conditions (FIG. 52(c), AUC washout=2937±1874 AU). Interestingly, FPG prior to the washout OGTT was significantly (p<0.05) decreased at 75±1 (11% decrease) compared to average FPG prior to device initiation.

It was next tested whether HFAC applied to the hepatic branch, without stimulation, (2 hrs of pre-treatment) would decrease glucose following an OGTT. It was found that there was no difference in glucose with HFAC alone (AUC=3143±805 AU) compared to pre-device initiation OGTTs. These results were similar to the results from the hepatic vagotomy alone treatment in the ZDF rat experiments as described in Example 5.

Using the same experimental design as Example 5, similar and significant glycemic control was observed in 3 non-Alloxan treated control pigs (FPG=68±3 mg/dL) with HFAC/stimulation. An OGTT prior to and following implantation of the devices demonstrated no significant difference in glycemic control due to device implant (pre-implant AUC=1582±451, pre-device initiation AUC=1164±172 p=0.41). However, following the initiation of HFAC/stimulation 2 hr prior to an OGTT there was a significant decrease in glucose compared to pre-device initiation (HFAC/stimulation AUC=583±131, p<0.05).

Modifications and equivalents of disclosed concepts such as those which might readily occur to one skilled in the art are intended to be included in the scope of the claims which are appended hereto. In addition, this disclosure contemplates application of a combination of electrical signal treatment by placement of electrodes on one or more nerves. This disclosure contemplates application of a therapy program to downregulate neural activity by application of an electrical signal treatment by placement of electrodes on one or more nerves. This disclosure contemplates application of a therapy program to upregulate neural activity by application of electrical signal treatment by placement of electrodes on one or more nerves. Any publications referred to herein are hereby incorporated by reference.

The above specification, examples and data provide a complete description of the manufacture and use of the composition of the invention. Since many embodiments of the invention can be made without departing from the spirit and scope of the invention, the invention resides in the claims hereinafter appended.

### Example 7—Effectiveness of Blocking/Stimulation Combination in Controlling Blood Glucose in Diabetic Swine

Experimental setup and procedures including the setup of high frequency blocking treatment and low frequency stimulation treatment were similar to Example 6 as described above. BLK/STIM controls consisted of oral administration of 75 grams of glucose to the diabetic swine without application of block and stimulation (BLK/STIM). Ingestion of a solution containing 75 grams of glucose is used clinically to diagnose severity of diabetes. Same amount of glucose was administered to the BLK/STIM group. Initiation of blocking/stimulation treatment was done at various time following glucose administration, and the blood glucose concentrations were measured at various time following the blocking/stimulation treatment.

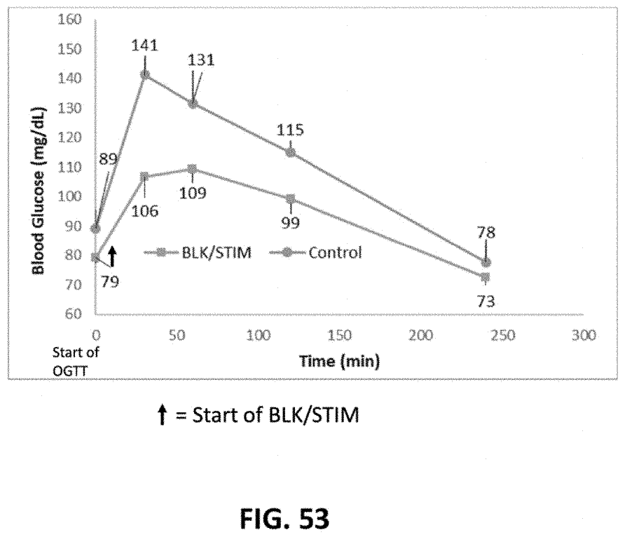

FIG. 53 shows the results of the BLK/STIM treatment initiated 5 minutes after glucose administration to the diabetic swine. The blocking signal is a continuous HFAC of 5,000 Hz. The low frequency stimulation signal is of 1 Hz. Compared with the control without application of the BLK/STIM signal, the blood glucose reaction is significantly lowered, with a reduction of peak value of blood glucose of about 23%.

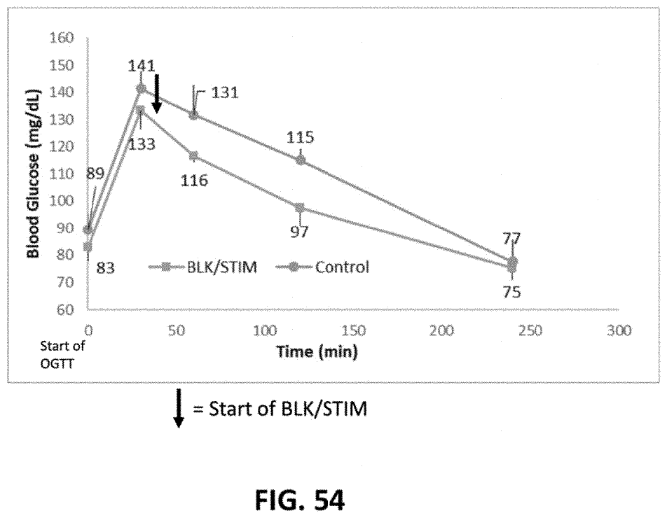

FIG. 54 shows the results of the BLK/STIM treatment initiated 30 minutes after glucose administration to the diabetic swine. Compared with the control without application of the BLK/STIM signal, the blood glucose reaction is considerably lower. However, the reduction of blood glucose over time is not as much as the reduction resulted from the BLK/STIM treatment initiated 5 minutes after glucose administration. The results indicate that the effectiveness of BLK/STIM treatment depends on the initiation time.

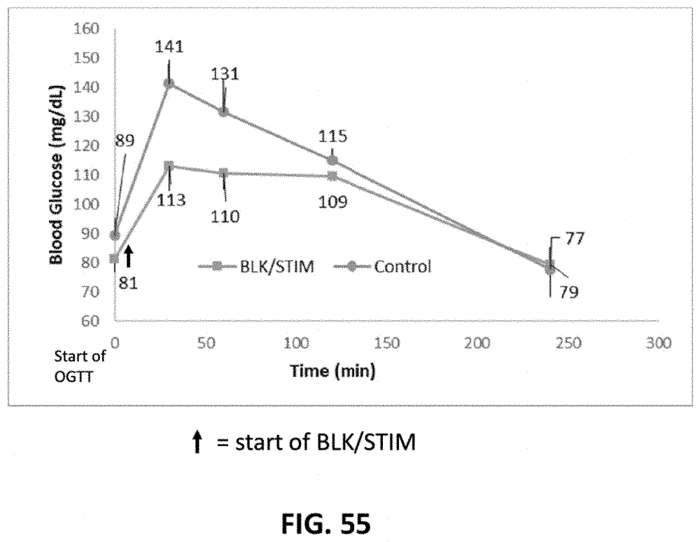

FIG. 55 shows the results of BLK/STIM treatment initiated 5 minutes after glucose administration to the diabetic swine. The blocking signal used herein is a 5,000 Hz HFAC having an intermittent pattern with millisecond active phase of 990 milliseconds and millisecond active phase of 10 milliseconds. The low frequency stimulation signal is of 1 Hz. Compared with the control without application of the BLK/STIM signal, the blood glucose reaction is significantly lowered, with a reduction of peak value of blood glucose of about 20%. These results indicate that both continuous and intermittent patterns of HFAC blocking signal can be combined with low frequency stimulation signals to effectively treat diabetic condition and control the blood glucose level.

Modifications and equivalents of disclosed concepts such as those which might readily occur to one skilled in the art are intended to be included in the scope of the claims which are appended hereto. In addition, this disclosure contemplates application of a combination of electrical signal treatment by placement of electrodes on one or more nerves. This disclosure contemplates application of a therapy program to downregulate neural activity by application of an electrical signal treatment by placement of electrodes on one or more nerves. This disclosure contemplates application of a therapy program to upregulate neural activity by application of electrical signal treatment by placement of electrodes on one or more nerves. Any publications referred to herein are hereby incorporated by reference.

The above specification, examples and data provide a complete description of the manufacture and use of the composition of the invention. Since many embodiments of the invention can be made without departing from the spirit and scope of the invention, the invention resides in the claims hereinafter appended.

1. A method for regulating nerve activity of a subject comprising:
applying a first electrical signal to a first nerve/organ and applying a second electrical signal to a second nerve/organ,
wherein the first electrical signal downregulates nerve activity and has a frequency from about 200 Hz to about 100 kHz, or from about 200 Hz to about 50 kHz, or from about 200 Hz to about 25 kHz, or from about 200 Hz to about 15 kHz, or from about 200 Hz to about 10 kHz, or from about 200 Hz to about 5,000, or from about 200 Hz to about 1,500 Hz, or from about 200 Hz to about 1,000 Hz, and
wherein the second electrical signal upregulates nerve activity and has a frequency from about 0.01 Hz to 199 Hz, or from about 0.01 Hz to about 100 Hz, or from about 0.01 Hz to about 50 Hz, or from about 0.01 Hz to about 30 Hz, or from about 0.01 Hz to about 10 Hz.

2. The method of claim 1, wherein the first electrical signal comprises at least one microsecond cycle and optionally a microsecond inactive phase, wherein each of the at least one microsecond cycle comprises at least one period, each of the at least one period comprising a pulse comprising a charge recharge phase, the pulse having a pulse width, and wherein the second electrical signal comprises at least one stimulation cycle, wherein each of the at least one stimulation cycle comprises at least one stimulation period, each of the at least one stimulation period comprising a pulse and optionally a stimulation inactive phase, wherein the pulse of the stimulation period comprises a cathodic phase and/or an anodic phase, and optionally a pulse delay, the pulse of the stimulation period having a pulse width.

3. The method of any of claim 1, wherein the first electrical signal further comprises at least one millisecond active phase, wherein each of the at least one millisecond active phase comprises at least one microsecond cycle, and wherein each of the at least one millisecond active phase is separated by a millisecond inactive phase.

4. The method of claim 1, wherein the second electrical signal further comprises at least one stimulation active phase, wherein each of the at least one stimulation active phase comprises at least one stimulation cycle, and wherein each of the at least one stimulation active phase is separated by an idle phase.

5. The method of claim 1, wherein the first electrical signal is low duty cycle of about 75% or less, or preferably 50% or less.

6-9. (canceled)

10. The method of claim 2, wherein the microsecond inactive phase of the first electrical signal is substantially longer than the period of the first electrical signal.

11. The method of claim 2, wherein the charge recharge phase of the first electrical signal further comprises a pulse delay between the charge and recharge phase thereof.

12. The method of claim 2, wherein the pulse of the second electrical signal is monophasic pulse, or biphasic pulse, or combinations thereof.

13-42. (canceled)

43. A system comprising:
an implantable neuroregulator;
at least one first electrode electrically connected to the implantable neuroregulator and adapted to be placed on a first nerve/organ; and
at least one second electrode electrically connected to the implantable neuroregulator and adapted to be placed on a second nerve/organ,
wherein the implantable neuroregulator comprises a microprocessor, the microprocessor configured to independently deliver a first electrical signal to the first nerve/organ through the first electrode and deliver a second electrical signal to the second nerve/organ through the second electrode,
wherein the first electrical signal has parameters to downregulate nerve activity and the second electrical signal has parameters to stimulate nerve activity, and wherein the first electrical signal has a frequency of about 200 Hz to about 100 kHz, wherein the second electrical signal has a frequency of about 0.01 Hz to 199 Hz.

44. The system of claim 43, wherein the first electrical signal is low duty cycle of about 75% or less, or about 50% or less.

45. The system of claim 43, wherein the first electrical signal comprises at least one microsecond cycle and optionally a microsecond inactive phase, wherein each of the at least one microsecond cycle comprises at least one period, each of the at least one period comprising a pulse comprising a charge recharge phase, the pulse having a pulse width, and wherein the second electrical signal comprises at least one stimulation cycle, wherein each of the at least one stimulation cycle comprises at least one stimulation period, each of the at least one stimulation period comprising a pulse and optionally a stimulation inactive phase, wherein the pulse of the stimulation period comprises a cathodic and/or anodic phase, and optionally a pulse delay, the pulse of the stimulation period having a pulse width.

46. The system of claim 43, wherein the first electrical signal further comprises at least one millisecond active phase, wherein each of the at least one millisecond active phase comprises at least one microsecond cycle, and wherein each of the at least one millisecond active phase is separated by a millisecond inactive phase.

47-51. (canceled)

52. The system of claim 43, wherein the microsecond inactive phase of the first electrical signal is substantially longer than the period of the first electrical signal.

53. The system of claim 43, wherein the charge recharge phase of the first electrical signal further comprises a pulse delay between the charge and recharge phase thereof.

54. The system of claim 43, wherein the pulse of the second electrical signal is monophasic pulse, or biphasic pulse, or combinations thereof.

55. The system of claim 43, wherein the first electrical signal and the second electrical signal each independently has an on time of about 30 seconds to about 30 minutes.

56. The system of claim 43, wherein the first electrical signal and the second electrical signal each independently has a current amplitude in a range from about 0.01 mAmps to about 20 mAmps.

57. The system of claim 43, wherein the first electrical signal and the second electrical signal each independently has a voltage in a range from about 0.01 volts to about 20 volts.

58-65. (canceled)

66. The system of claim 43, wherein the subject has a disease or disorder selected from the group consisting of obesity, overweight, pancreatitis, dysmotility, bulimia, gastrointestinal disease with an inflammatory basis, ulcerative colitis, Crohn's disease, low vagal tone, gastroparesis, diabetes, prediabetes, Type II diabetes, chronic pain, hypertension, gastroesophageal reflux disease, peptic ulcer disease and combinations thereof.

67. The system of claim 43, wherein the first nerve and the second nerve are independently from a nerve selected from the group consisting of the vagus nerve, anterior vagus nerve, posterior vagus nerve, hepatic branch of vagus nerve, celiac branch of vagus nerve, renal nerve, renal artery, sympathetic nerves, baroreceptors, glossopharyngeal nerve, and combinations thereof.

68. The system of claim 43, wherein the first organ and the second organ are selected from the group of duodenum, jejunum, ileum, small bowel, colon, stomach, esophagus, liver, spleen, pancreas, and combinations thereof.

69-76. (canceled)

77. A system for treating a condition associated with impaired blood glucose regulation comprising:
an implantable neuroregulator;
at least one first electrode electrically connected to the implantable neuroregulator and adapted to be placed on one or more hepatic nerve branch of a vagus nerve or any segment of the anterior vagus nerve cranial to the hepatic branch of a subject;
at least one second electrode electrically connected to the implantable neuroregulator and adapted to be placed on one or more celiac nerve branch of the vagus nerve or any segment of the posterior vagus nerve cranial to the celiac branch of the subject; and
a blood glucose sensor configured to measure the blood glucose of the subject and convey a blood glucose value to the system,
wherein the implantable neuroregulator comprises a microprocessor, wherein the microprocessor is configured to independently deliver a first electrical signal to the hepatic nerve branch through the first electrode and deliver a second electrical signal to the celiac branch through the second electrode, wherein the first electrical signal has parameters to downregulate nerve activity and the second electrical signal has parameters to stimulate nerve activity, and wherein the first electrical signal has a frequency of about 200 Hz to about 100 kHz, wherein the second electrical signal has a frequency of about 0.01 Hz to 199 Hz,
and wherein the microprocessor is configured to apply a coordinated change to the first electrical signal and/or the second electrical signal in response to the blood glucose value.

78-82. (canceled)
